(19) United States
(12) Patent Application Publication
(10) Pub. No.: US 2019/0330167 A1
(43) Pub. Date: Oct. 31, 2019

(54) ACTIVATORS OF HIV LATENCY

(71) Applicants: The Walter and Eliza Hall Institute of Medical Research, Victoria (AU); The University of Melboume, Victoria (AU)

(72) Inventors: Brad SLEEBS, Victoria (AU); Damian Francis John PURCELL, Victoria (AU); Jonathan JACOBSON, Victoria (AU); Sharon LEWIN, Victoria (AU); William NGUYEN, Victoria (AU)

(21) Appl. No.: 16/310,646

(22) PCT Filed: Jun. 21, 2017
(86) PCT No.: PCT/AU2017/050631

§ 371 Date: Dec. 17, 2018

(30) Foreign Application Priority Data
Jun. 21, 2016 (AU) 2016902426

(51) Int. Cl. C07D 277/46; C07D 263/48; C07D 213/75; C07D 285/135; C07D 249/14; C07D 271/113; C07D 231/40; C07D 261/14; C07D 471/04; C07D 417/12; A61P 31/18; A61K 31/551; A61K 31/496; A61K 31/437; A61K 31/42; A61K 31/415; A61K 31/4245; A61K 31/4196; A61K 31/433; A61K 31/4402; A61K 31/421; A61K 31/426

(52) U.S. Cl. CPC C07D 277/46; A61K 31/426; C07D 213/75; C07D 285/135; C07D 249/14; C07D 271/113; C07D 231/40; C07D 261/14; C07D 471/04; C07D 417/12; A61P 31/18; A61K 31/551; A61K 31/496; A61K 31/437; A61K 31/42; A61K 31/415; A61K 31/4245; A61K 31/4196; A61K 31/433; A61K 31/4402; A61K 31/421; C07D 263/48

(57) ABSTRACT

The present invention relates to novel compounds which active HIV expression in latently infected cells. More particularly, the invention relates to pharmaceutical compositions comprising the novel compounds and their use in activating HIV expression in latently infected cells. Further still, the invention relates to pharmaceutical compositions comprising the novel compounds in combination with anti-HIV therapy compounds and their use in treating HIV infection in both animals and humans. The invention further provides means for preparing the compounds.

### FIELD OF THE INVENTION

The present invention relates to novel compounds which activate HIV expression in latently infected cells. More particularly, the invention relates to pharmaceutical compositions comprising the novel compounds and their use in activating HIV expression in latently infected cells. Further still, the invention relates to pharmaceutical compositions comprising the novel compounds in combination with anti-HIV therapy compounds and their use in treating HIV infection in both animals and humans. The invention further provides means for preparing the compounds.

### BACKGROUND OF THE INVENTION

Treatment of HIV-1 infection with combination antiretroviral therapy (cART) has dramatically reduced mortality, and life expectancy is now normal for a person living with HIV. However treatment must be taken lifelong and there is no cure. If cART is stopped, virus will rebound to pre-treatment levels within 2-3 weeks due to the enduring presence of long-lived, latently infected CD4+ T-cells and other reservoirs (Deeks 2013; Lewin 2014). Current cART eliminates active virus replication but has no activity against latent HIV infection. Latency is a common feature of many viruses, but with HIV, occurs when the virus is able to enter and integrate proviral DNA into the host genome but doesn't produce progeny virus to complete the viral replication cycle. However, following certain stimuli, infectious virus can be released. A latently infected cell usually does not express viral proteins and consequently is invisible to immune recognition.

One strategy to eliminate latently infected cells is to specifically activate latent virus to reveal its location in scarce cells so they can be successfully treated with cART. Compounds known to activate latent HIV, generically called latency reversing agents (LRAs), include T-cell mitogens such as phorbol myristate acetate (PMA) and phytohaemaqglutinin (PHA), protein kinase C (PKC) agonists, bromodomain inhibitors such as JQ1 (+) and/or epigenetic modifying drugs, including histone deacetylase (HDAC) inhibitors. However, mitogens, PKC agonists and HDAC inhibitors lack specificity for latent HIV and modify gene expression of a large number of host genes (Archin 2012; Elliot 2014). In addition, these drugs have multiple potential adverse effects.

There is therefore an unmet need to specifically activate HIV expression in latently infected cells to reveal its location in scarce cells, but in a manner that does not disrupt normal cell gene expression.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a compound of Formula (I):

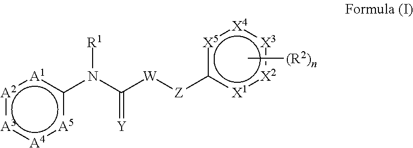

or a salt, solvate, or prodrug thereof

wherein

A¹, A², A³, A⁴ and A⁵ are independently selected from the group consisting of CR′, NR″, O and S, wherein A⁵ may or may not be present;

R′ is selected from the group consisting of H, C₁-C₄alkyl, O(C₁-C₄alkyl), CONR⁵R⁶, halo, CF₃, CF₂H and CN;

R″ is selected from H and C₁-C₄alkyl, wherein R″ may or may not be present;

R¹ is selected from H and C₁-C₄alkyl;

Y is selected from O and NH;

wherein when Y is NH and A⁵ is CH, optionally Y and A⁵ together form an imidazole ring so that the compound has the structure:

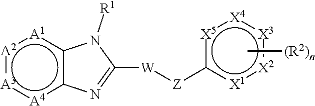

W is selected from the group consisting of C₁-C₄alkyl, NH, N(C₁-C₄alkyl) and O;

Z is selected from the group consisting of C₁-C₄alkyl, (CH₂)ₘO, (CH₂)ₘNH, (CH₂)ₘN(CH₃), and m is 0 or 1, wherein when W is O, m is 1;

alternatively W and Z together form an optionally substituted piperazine or piperidine ring so that the compound has the structure:

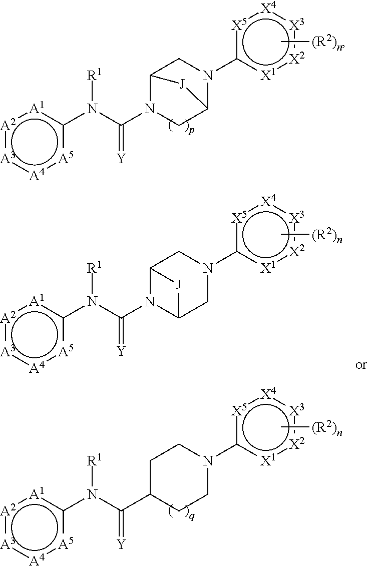

J is selected from CH₂ and (CH₂)₂, wherein J may or may not be present, p is 1 or 2, and q is 0 or 1;

X¹, X², X³, X⁴ and X⁵ are independently selected from the group consisting of CH, N, NH, O and S, wherein X⁵ may or may not be present;

each R² is independently selected from the group consisting of C₁-C₄alkyl, CN, CF₃, F, Cl, Br, hydroxyl, nitro, OR⁶, COR⁶, CO₂R⁶, CONR⁵R⁶, CONHSO₂R⁵, SO₂NHCOR⁵, CONR⁵OR⁶, C₁-C₄alkylNR⁵R⁶, C₁-C₄alkylOR⁶, NR⁵R⁶, NR⁵COR⁶, NR⁷CONR⁵R⁶ and NR⁵CO₂R⁶;

n is 0-3;

R⁵ and R⁶ are independently selected from the group consisting of H, C₁-C₄alkyl, C₃-C₁₀cycloalkyl, C₃-C₁₀heterocyclyl, C₆-C₁₀aryl, C₅-C₁₀heteroaryl, (C₁-C₄alkyl)C₆-C₁₀aryl and (C₁-C₄alkyl)C₅-C₁₀heteroaryl;

alternatively when R⁵ and R⁶ are bound to the same atom they form an optionally substituted C₃-C₁₀cycloalkyl or C₃-C₁₀heterocyclyl;

R⁷ is selected from H and CH₃.

Certain of the compounds of formula I are previously know, however, their use in methods of activating latent HIV virus in cells is surprising. Many of the compounds of formula I have not previously been known.

In one aspect, there is provided a composition comprising a compound according to Formula (I) or a salt, solvate, or prodrug thereof, and a pharmaceutically acceptable excipient.

In another aspect, there is provided a method for activating HIV expression in latently infected cells in a subject in need thereof, the method comprising administering an effective amount of a compound or a salt, solvate, or prodrug thereof of Formula (I) to the subject.

In another aspect, there is provided a method for activating HIV expression in latently infected cells in a subject in need thereof, the method comprising administering an effective amount of a composition comprising a compound or a salt, solvate, or prodrug thereof of Formula (I) to the subject.

In another aspect, there is provided a method for treating HIV infection in a subject in need thereof, the method comprising administering an effective amount of a compound or a salt, solvate, or prodrug thereof of Formula (I) in combination with a therapeutically effective amount of one or more anti-HIV viral therapy compounds to the subject.

In another aspect, there is provided a method for treating HIV infection in a subject in need thereof, the method comprising administering an effective amount of a composition comprising a compound or a salt, solvate, or prodrug thereof of Formula (I) in combination with a therapeutically effective amount of one or more anti-HIV viral therapy compounds to the subject.

In another aspect, there is provided use of a compound of Formula (I) or a salt, solvate, or prodrug thereof for activating HIV expression in latently infected cells in a subject in need thereof.

In another aspect, there is provided use of a composition comprising a compound of Formula (I) or a salt, solvate, or prodrug thereof for activating HIV expression in latently infected cells in a subject in need thereof.

In another aspect, there is provided use of a compound of Formula (I) or a salt, solvate, or prodrug thereof in combination with one or more anti-HIV viral therapy compounds for treating HIV infection in a subject in need thereof.

In another aspect, there is provided use of a composition comprising a compound of Formula (I) or a salt, solvate, or prodrug thereof in combination with one or more anti-HIV viral therapy compounds for treating HIV infection in a subject in need thereof.

In yet another aspect, there is provided a compound according to Formula (I) or a salt, solvate, or prodrug thereof for use in activating HIV expression in latently infected cells in a subject in need thereof.

In yet another aspect, there is provided a composition comprising a compound according to Formula (I) or a salt, solvate, or prodrug thereof for use in activating HIV expression in latently infected cells in a subject in need thereof.

In yet another aspect, there is provided a compound according to Formula (I) or a salt, solvate, or prodrug thereof in combination with one or more anti-HIV viral therapy compounds for use in treating HIV infection in a subject in need thereof.

In yet another aspect, there is provided a composition comprising a compound according to Formula (I) or a salt, solvate, or prodrug thereof in combination with one or more anti-HIV viral therapy compounds for use in treating HIV infection in a subject in need thereof.

In yet another aspect, there is provided a compound according to Formula (I) or a salt, solvate, or prodrug thereof when used for activating HIV expression in latently infected cells in a subject in need thereof.

In yet another aspect, there is provided a composition comprising a compound according to Formula (I) or a salt, solvate, or prodrug thereof when used for activating HIV expression in latently infected cells in a subject in need thereof.

In yet another aspect, there is provided a compound according to Formula (I) or a salt, solvate, or prodrug thereof in combination with one or more anti-HIV viral therapy compounds when used for treating HIV infection in a subject in need thereof.

In yet another aspect, there is provided a composition comprising a compound according to Formula (I) or a salt, solvate, or prodrug thereof in combination with one or more anti-HIV viral therapy compounds when used for treating HIV infection in a subject in need thereof.

Any embodiment herein shall be taken to apply mutatis mutandis to any other embodiment unless specifically stated otherwise.

The present disclosure is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. The luminescence output of HIV-1 long terminal repeat driven luciferase reporter gene expression in FlipIn.FM HEK293 cells (which represents reactivation of HIV expression) as a function of the concentration of Compound 1.

FIG. 2. The luminescence output of HIV-1 long terminal repeat driven luciferase reporter gene expression in FlipIn.FM HEK293 cells (which represents reactivation of HIV expression) as a function of the concentration of Compound 2.

FIG. 3. The luminescence output of HIV-1 long terminal repeat driven luciferase reporter gene expression in FlipIn.FM HEK293 cells (which represents reactivation of HIV expression) as a function of the concentration of Compound 3.

FIG. 4. The luminescence output of HIV-1 long terminal repeat driven luciferase reporter gene expression in FlipIn.FM HEK293 cells (which represents reactivation of HIV expression) as a function of the concentration of Compound 4.

FIG. 5. The luminescence output of HIV-1 long terminal repeat driven luciferase reporter gene expression in FlipIn.FM HEK293 cells (which represents reactivation of HIV expression) as a function of the concentration of Compound 5.

FIG. 6. The luminescence output of HIV-1 long terminal repeat driven luciferase reporter gene expression in FlipIn.FM HEK293 cells (which represents reactivation of HIV expression) as a function of the concentration of Compound 6.

FIG. 7. The luminescence output of HIV-1 long terminal repeat driven luciferase reporter gene expression in FlipIn.FM HEK293 cells (which represents reactivation of HIV expression) as a function of the concentration of Compound 7.

FIG. 8. The luminescence output of HIV-1 long terminal repeat driven luciferase reporter gene expression in FlipIn.FM HEK293 cells (which represents reactivation of HIV expression) as a function of the concentration of Compound 8.

FIG. 9. The luminescence output of HIV-1 long terminal repeat driven luciferase reporter gene expression in FlipIn.FM HEK293 cells (which represents reactivation of HIV expression) as a function of the concentration of Compound 9.

FIG. 10. The luminescence output of HIV-1 long terminal repeat driven luciferase reporter gene expression in FlipIn.FM HEK293 cells (which represents reactivation of HIV expression) as a function of the concentration of Compound 10.

FIG. 11. The luminescence output of HIV-1 long terminal repeat driven luciferase reporter gene expression in FlipIn.FM HEK293 cells (which represents reactivation of HIV expression) as a function of the concentration of Compound 11.

FIG. 12. The luminescence output of HIV-1 long terminal repeat driven luciferase reporter gene expression in FlipIn.FM HEK293 cells (which represents reactivation of HIV expression) as a function of the concentration of Compound 12.

FIG. 13. The luminescence output of HIV-1 long terminal repeat driven luciferase reporter gene expression in FlipIn.FM HEK293 cells (which represents reactivation of HIV expression) as a function of the concentration of Compound 13.

FIG. 14. The luminescence output of HIV-1 long terminal repeat driven luciferase reporter gene expression in FlipIn.FM HEK293 cells (which represents reactivation of HIV expression) as a function of the concentration of Compound 14.

FIG. 15. The luminescence output of HIV-1 long terminal repeat driven luciferase reporter gene expression in FlipIn.FM HEK293 cells (which represents reactivation of HIV expression) as a function of the concentration of Compound 15.

FIG. 16. The luminescence output of HIV-1 long terminal repeat driven luciferase reporter gene expression in FlipIn.FM HEK293 cells (which represents reactivation of HIV expression) as a function of the concentration of Compound 16.

FIG. 17. The luminescence output of HIV-1 long terminal repeat driven luciferase reporter gene expression in FlipIn.FM HEK293 cells (which represents reactivation of HIV expression) as a function of the concentration of Compound 17.

FIG. 18. The luminescence output of HIV-1 long terminal repeat driven luciferase reporter gene expression in FlipIn.FM HEK293 cells (which represents reactivation of HIV expression) as a function of the concentration of Compound 18.

FIG. 19. The luminescence output of HIV-1 long terminal repeat driven luciferase reporter gene expression in FlipIn.FM HEK293 cells (which represents reactivation of HIV expression) as a function of the concentration of Compound 19.

FIG. 20. The luminescence output of HIV-1 long terminal repeat driven luciferase reporter gene expression in FlipIn.FM HEK293 cells (which represents reactivation of HIV expression) as a function of the concentration of Compound 20.

FIG. 21. The luminescence output of HIV-1 long terminal repeat driven luciferase reporter gene expression in FlipIn.FM HEK293 cells (which represents reactivation of HIV expression) as a function of the concentration of Compound 21.

FIG. 22. The luminescence output of HIV-1 long terminal repeat driven luciferase reporter gene expression in FlipIn.FM HEK293 cells (which represents reactivation of HIV expression) as a function of the concentration of Compound 22.

FIG. 23. The luminescence output of HIV-1 long terminal repeat driven luciferase reporter gene expression in FlipIn.FM HEK293 cells (which represents reactivation of HIV expression) as a function of the concentration of Compound 23.

FIG. 24. The luminescence output of HIV-1 long terminal repeat driven luciferase reporter gene expression in FlipIn.FM HEK293 cells (which represents reactivation of HIV expression) as a function of the concentration of Compound 24.

FIG. 25. The luminescence output of HIV-1 long terminal repeat driven luciferase reporter gene expression in FlipIn.FM HEK293 cells (which represents reactivation of HIV expression) as a function of the concentration of Compound 25.

FIG. 26. The luminescence output of HIV-1 long terminal repeat driven luciferase reporter gene expression in FlipIn.FM HEK293 cells (which represents reactivation of HIV expression) as a function of the concentration of Compound 26.

FIG. 27. The luminescence output of HIV-1 long terminal repeat driven luciferase reporter gene expression in FlipIn.FM HEK293 cells (which represents reactivation of HIV expression) as a function of the concentration of Compound 27.

FIG. 28. The luminescence output of HIV-1 long terminal repeat driven luciferase reporter gene expression in FlipIn.FM HEK293 cells (which represents reactivation of HIV expression) as a function of the concentration of Compound 28.

FIG. 29. The luminescence output of HIV-1 long terminal repeat driven luciferase reporter gene expression in FlipIn.FM HEK293 cells (which represents reactivation of HIV expression) as a function of the concentration of Compound 29.

FIG. 30. The luminescence output of HIV-1 long terminal repeat driven luciferase reporter gene expression in FlipIn.FM HEK293 cells (which represents reactivation of HIV expression) as a function of the concentration of Compound 30.

FIG. 31. The luminescence output of HIV-1 long terminal repeat driven luciferase reporter gene expression in FlipIn.FM HEK293 cells (which represents reactivation of HIV expression) as a function of the concentration of Compound 31.

FIG. 32. The luminescence output of HIV-1 long terminal repeat driven luciferase reporter gene expression in FlipIn.FM HEK293 cells (which represents reactivation of HIV expression) as a function of the concentration of Compound 32.

FIG. 33. The luminescence output of HIV-1 long terminal repeat driven luciferase reporter gene expression in FlipIn.FM HEK293 cells (which represents reactivation of HIV expression) as a function of the concentration of Compound 33.

FIG. 34. The luminescence output of HIV-1 long terminal repeat driven luciferase reporter gene expression in FlipIn.FM HEK293 cells (which represents reactivation of HIV expression) as a function of the concentration of Compound 34.

FIG. 35. The luminescence output of HIV-1 long terminal repeat driven luciferase reporter gene expression in FlipIn.FM HEK293 cells (which represents reactivation of HIV expression) as a function of the concentration of Compound 35.

FIG. 36. The luminescence output of HIV-1 long terminal repeat driven luciferase reporter gene expression in FlipIn.FM HEK293 cells (which represents reactivation of HIV expression) as a function of the concentration of Compound 36.

FIG. 37. The luminescence output of HIV-1 long terminal repeat driven luciferase reporter gene expression in FlipIn.FM HEK293 cells (which represents reactivation of HIV expression) as a function of the concentration of Compound 37.

FIG. 38. The luminescence output of HIV-1 long terminal repeat driven luciferase reporter gene expression in FlipIn.FM HEK293 cells (which represents reactivation of HIV expression) as a function of the concentration of Compound 38.

FIG. 39. The luminescence output of HIV-1 long terminal repeat driven luciferase reporter gene expression in FlipIn.FM HEK293 cells (which represents reactivation of HIV expression) as a function of the concentration of Compound 39.

FIG. 40. The relative luminescence output of HIV-1 long terminal repeat (LTR) driven click beetle red (CBR) luciferase reporter gene expression in FlipIn.FM HEK293 cells (which represents reactivation of HIV expression) and the luminescence output of complimentary off-target cytomegalovirus (CMV) immediate early promoter driven click beetle green (CBG) luciferase reporter (which represents global gene activation) as a function of the concentration of Compounds 1, 6 and 39 on the left and the activation of the HIV LTR-driven green fluorescent protein (GFP) reporter in the J-Lat10.6 model of T-cell HIV-latency using cells incorporating a non-specific CMV driven red fluorescent reporter (dsRED) that is a measure of off-target gene activation.

FIG. 41a. Induction of HIV-1 gene expression in leukapheresis samples using known HIV latency reversing agents (LRAs) and compounds according to the present invention. Resting memory CD4+ T cells isolated by leukapheresis from HIV+ donors on antiretroviral therapy were reactivated for 72 hrs using known LRAs including vorinostat (Vor) also known as suberanilohydroxamic acid, another hydroxamic acid panobinostat (Pan), and the depsipeptide Romidepsin (Rom) that are all HDACi, together with JQ1 (+) which is a thienotriazolodiazepine known to inhibit the BET family of bromodomain proteins including BRD2, BRD3, and BRD4, and compared with compounds according to the present invention DP#6 (Compound 41), DP#14 (Compound 7), and DP#16 (Compound 64). The graph shows HIV-1 RNA detected through qPCR as an absolute number of copies of HIV-1 RNA per 125 ng of whole cell RNA is shown, together with unstimulated cells (Unstim), vehicle dimethylsulphoxide (DMSO) negative control, and phorbol myristate acetate (PMA) mitogen positive control. Error bars represent standard deviation of n=4 or n=5 donors.

FIG. 41b. Induction of HIV-1 gene expression in leukapheresis samples using known LRAs and compounds according to the present invention. Resting memory CD4+ T cells isolated by leukapheresis from HIV+ donors on antiretroviral therapy were reactivated for 72 hrs using known (vorinostat, Vor; panobinostat, Pan; romidepsin, Rom; and JQ1 (+)) and compounds according to the present invention (DP#6 (Compound 41), DP#14 (Compound 7), and DP#16 (Compound 64)). The graph shows HIV-1 RNA detected through qPCR as fold change over the unstimulated baseline together with unstimulated cells (Unstim), vehicle dimethylsulphoxide (DMSO) negative control, and phorbol myristate acetate (PMA) mitogen positive control. Error bars represent standard deviation of n=4 or n=5 donors.

FIG. 41c. Induction of HIV-1 gene expression in leukapheresis samples using known LRAs and compounds according to the present invention. Resting memory CD4+ T cells isolated by leukapheresis from HIV+ donors on antiretroviral therapy were reactivated for 72 hrs using known (vorinostat, Vor; panobinostat, Pan; romidepsin, Rom; and JQ1 (+)) and compounds according to the present invention (DP#6 (Compound 41), DP#14 (Compound 7), and DP#16 (Compound 64)). The graph shows HIV-1 RNA detected through qPCR as values normalized between the unstimulated baseline and phorbol myristate acetate (PMA) mitogen activated positive control as 100%. Error bars represent standard deviation of n=4 or n=5 donors.

FIG. 42a. Synergystic induction of HIV-1 gene expression in leukapheresis samples using known LRAs with DP#14 (Compound 7). Resting memory CD4+ T cells isolated by leukapheresis from HIV+ donors on antiretroviral therapy were reactivated for 72 hrs using known (JQ1 (+)) and novel LRAs (DP#14 (Compound 7)) alone and in combination, and HIV-1 RNA detected through qPCR. An absolute number of copies of HIV-1 RNA per 125 ng of whole cell RNA is shown, together with unstimulated cells (Unstim), vehicle dimethylsulphoxide (DMSO) negative control, and phorbol myristate acetate (PMA) mitogen positive control. Error bars represent standard deviation of n=4 or n=5 donors.

FIG. 42b. Synergystic induction of HIV-1 gene expression in leukapheresis samples using known LRAs with DP#14 (Compound 7). Resting memory CD4+ T cells isolated by leukapheresis from HIV+ donors on antiretroviral therapy were reactivated for 72 hrs using known (JQ1 (+)) and compounds according to the present invention (DP#14 (Compound 7)) alone and in combination, and HIV-1 RNA detected through qPCR. Fold change over the unstimulated baseline is shown for unstimulated cells (Unstim), vehicle dimethylsulphoxide (DMSO) negative control, and phorbol myristate acetate (PMA) mitogen positive control. Error bars represent standard deviation of n=4 or n=5 donors.

FIG. 42c. Induction of HIV-1 gene expression in leukapheresis samples using known LRAs and DP#14 (Compound 7). Resting memory CD4+ T cells isolated by leukapheresis from HIV+ donors on antiretroviral therapy were reactivated for 72 hrs using known (JQ1 (+)) and compounds according to the present invention (DP#14 (Compound 7)) alone and in combination, and HIV-1 RNA detected through qPCR. The graph shows HIV-1 RNA detected through qPCR as values normalized between the unstimulated baseline and phorbol myristate acetate (PMA) mitogen activated positive control as 100%. Error bars represent standard deviation of n=4 or n=5 donors.

FIG. 43. Enhanced induction of HIV-1 gene expression in the J.Lat10.6 cell line model using compounds according to the present invention. The J.Lat10.6 latently infected T-cell line was treated with 4 compounds of the present invention, DP#6 (Compound 41), DP#14 (Compound 7), DP#18 (Compound 65), and DP#19 (Compound 73) and reactivated for 48 hrs. HIV-1 reactivation was measured by flowcytometry for GFP expression. Error bars represent standard deviation of n=3 experiments.

FIG. 44. Synergistic reactivation with JQ1 (+) and DP#14 (Compound 7) in FlipIn.FM model. The FlipIn.FM model of HIV-1 latency was treated with JQ1 (+) and DP#14 (Compound 7) alone at 10 μM and in combination, and the HIV-1 gene expression measured 48 hrs post treatment. Error bars represent standard deviation of n=3 experiments. OPTI represents media alone, without DP#14.

FIG. 45. Synergistic reactivation with JQ1 (+) and DP#14 (Compound 7) in J.Lat10.6 model. The J.Lat10.6 model of HIV-1 latency was treated with JQ1 (+) and DP#14 (Compound 7) alone at 10 μM and in combination, and the HIV-1 gene expression measured 48 hrs post treatment. Error bars represent standard deviation of n=3 experiments. OPTI represents media alone, without DP#14.

FIG. 46. Synergistic reactivation with PFI-1 (+) and DP#14 (Compound 7) in FlipIn.FM model. The FlipIn.FM model of HIV-1 latency was treated with PFI-1 and DP#14 alone at 10 μM and in combination, and the HIV-1 gene expression measured 48 hrs post treatment. Error bars represent standard deviation of n=3 experiments. OPTI represents media alone, without DP#14.

FIG. 47. Synergistic reactivation with PFI-1 (+) and DP#14 (Compound 7) in J.Lat10.6 model. The J.Lat10.6 model of HIV-1 latency was treated with PFI-1 and DP#14 alone at 10 μM and in combination, and the HIV-1 gene expression measured 48 hrs post treatment. Error bars represent standard deviation of n=3 experiments. OPTI represents media alone, without DP#14.

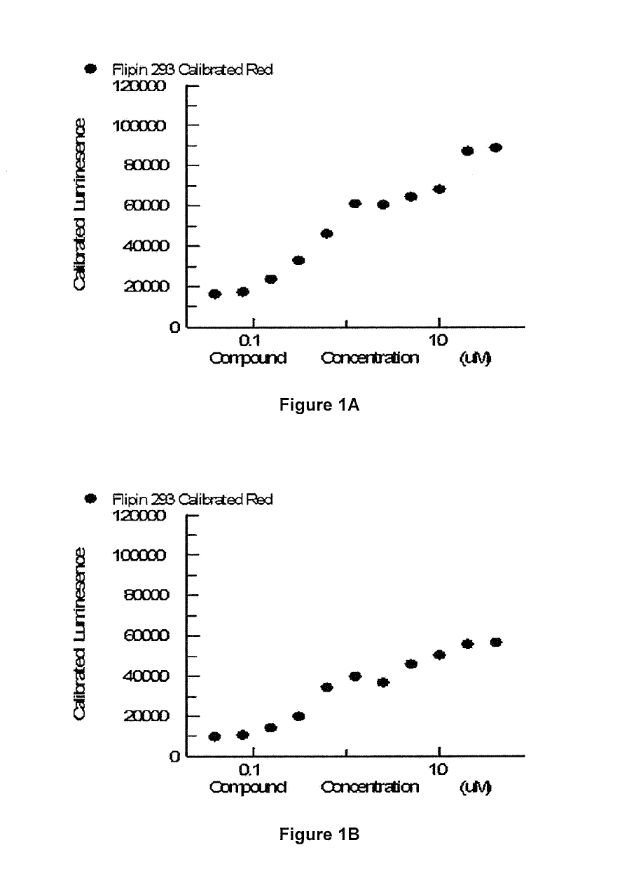

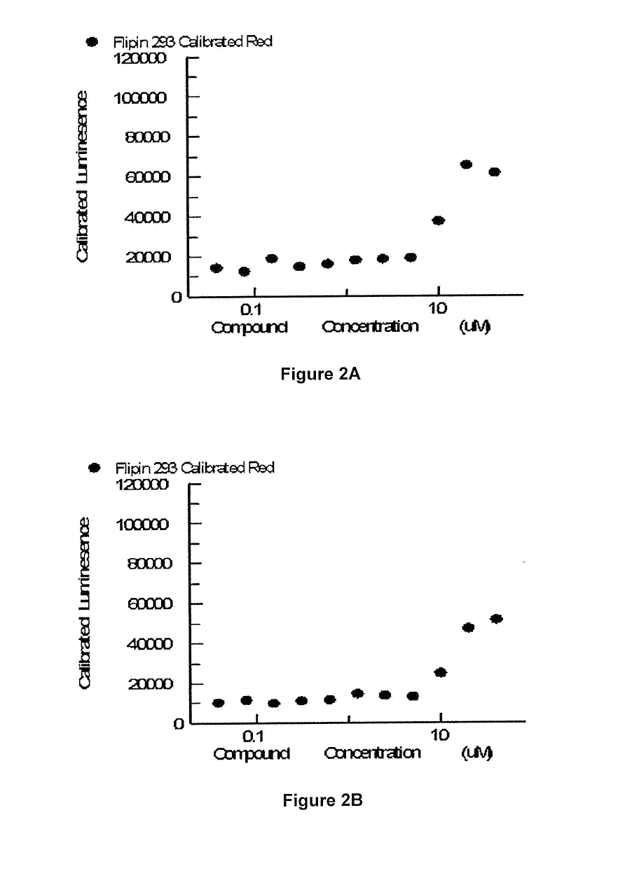

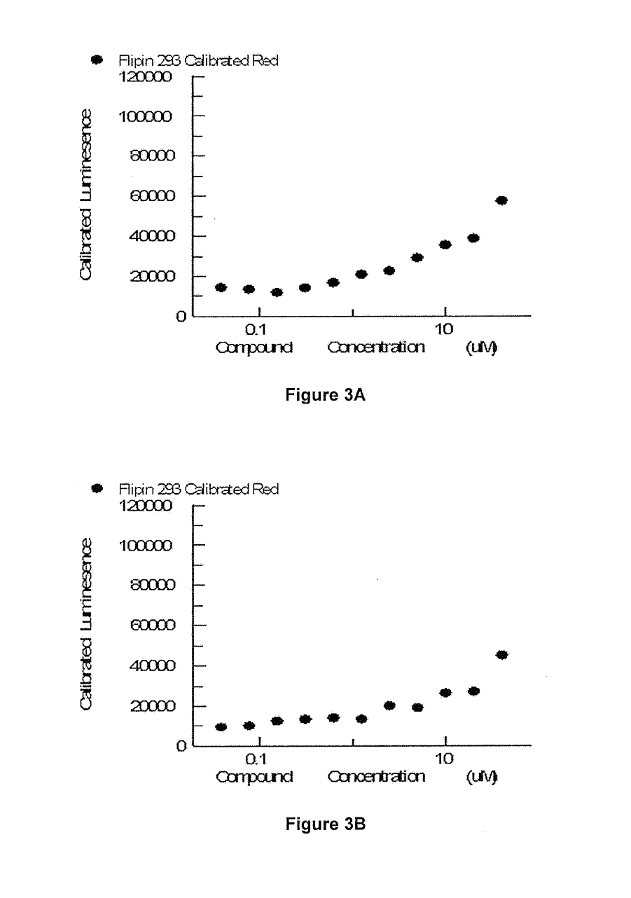

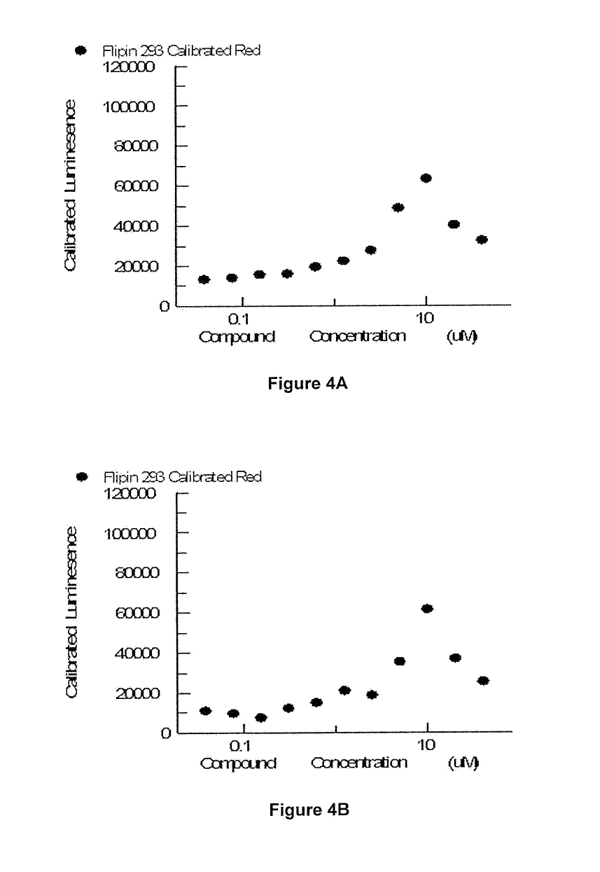

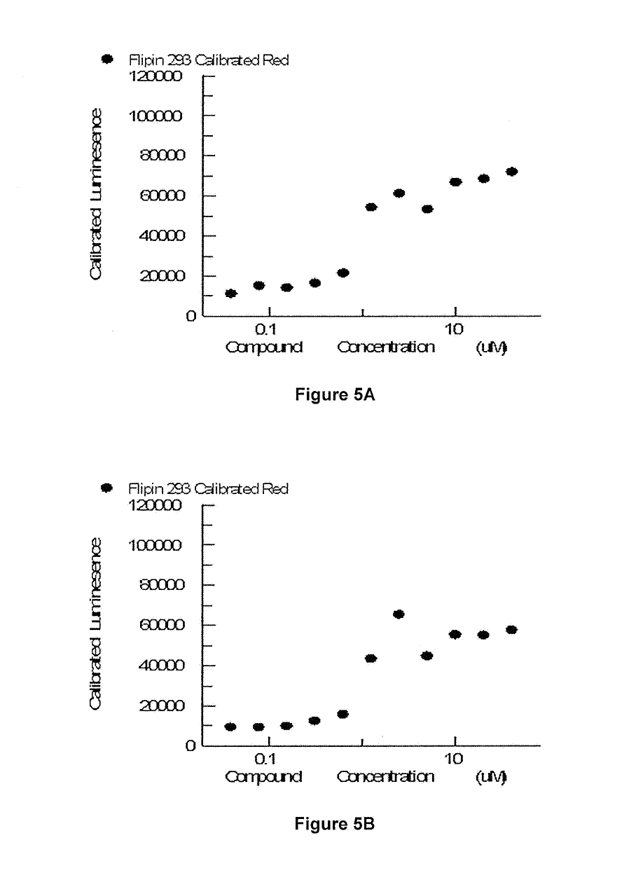

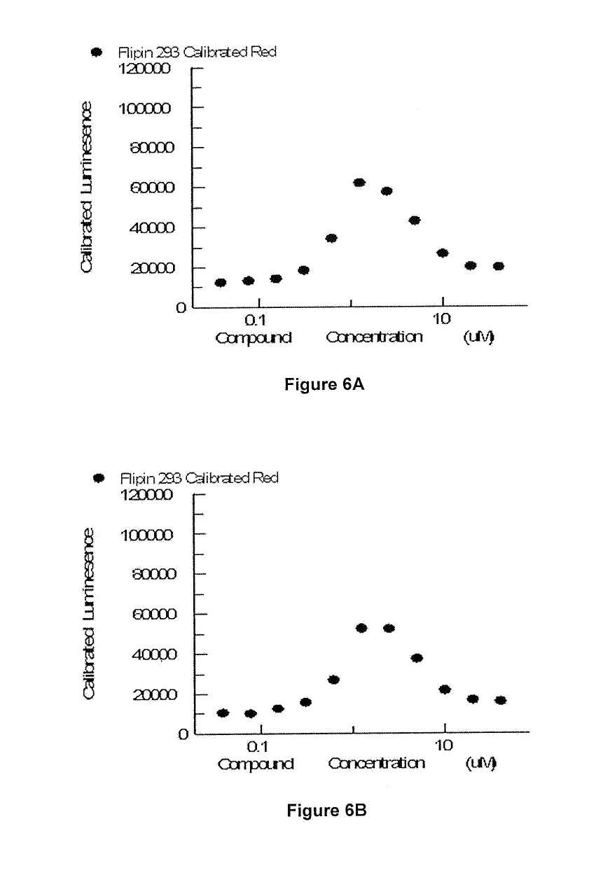

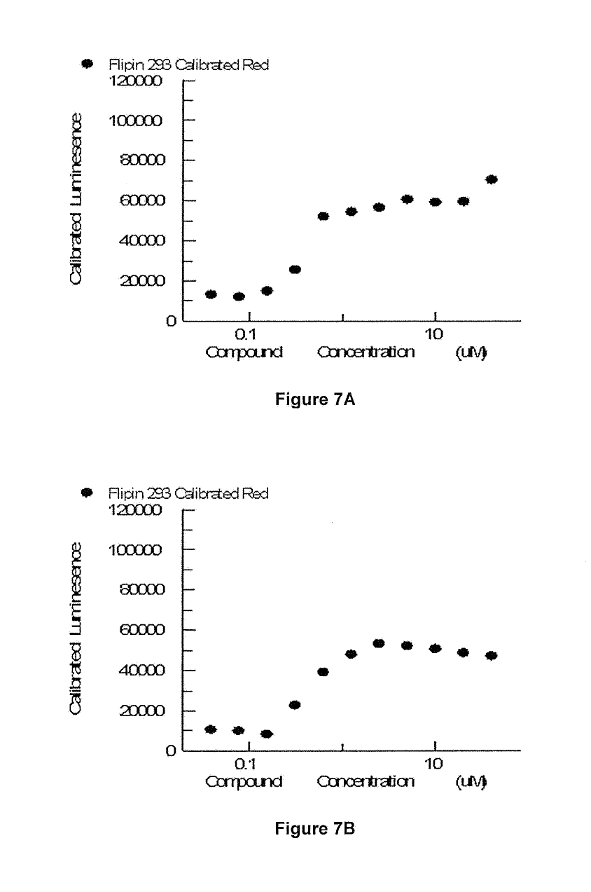

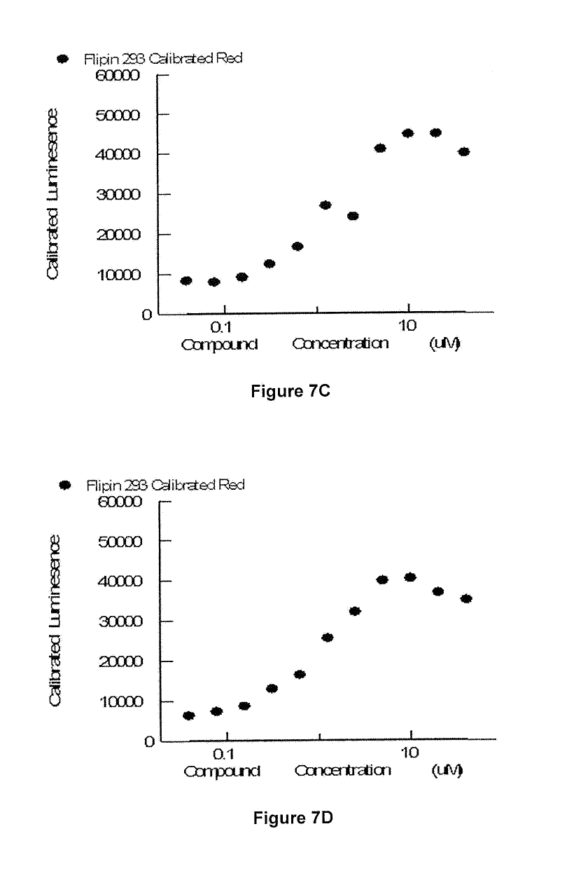

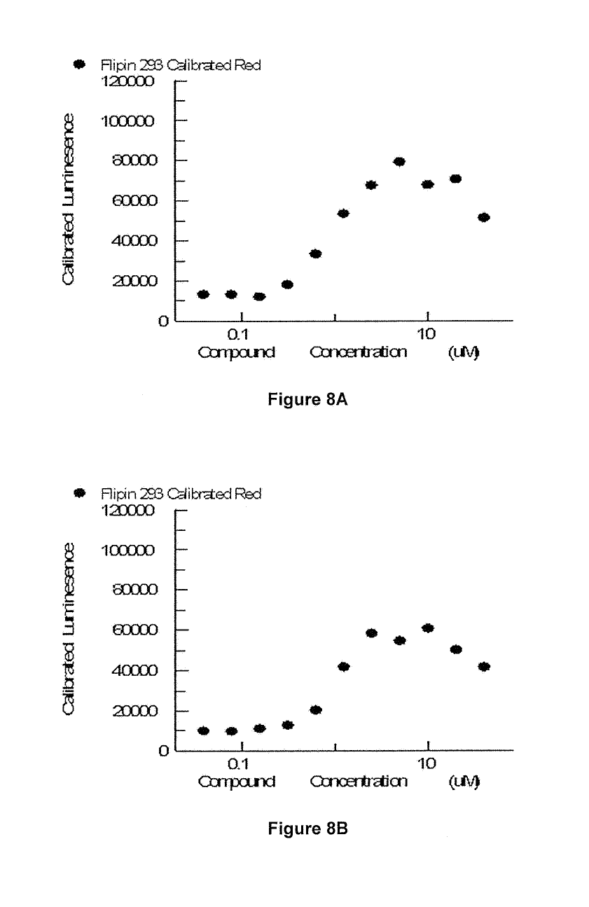

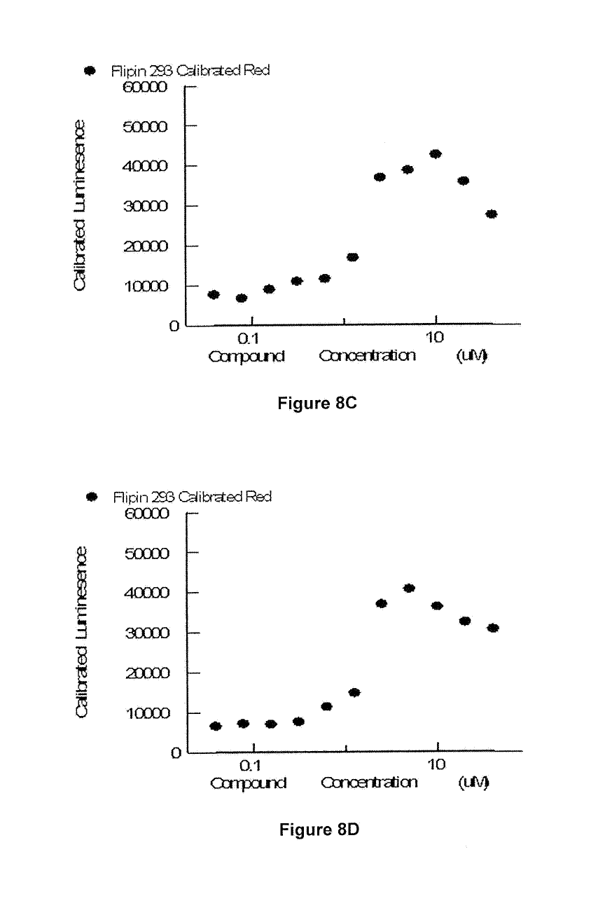

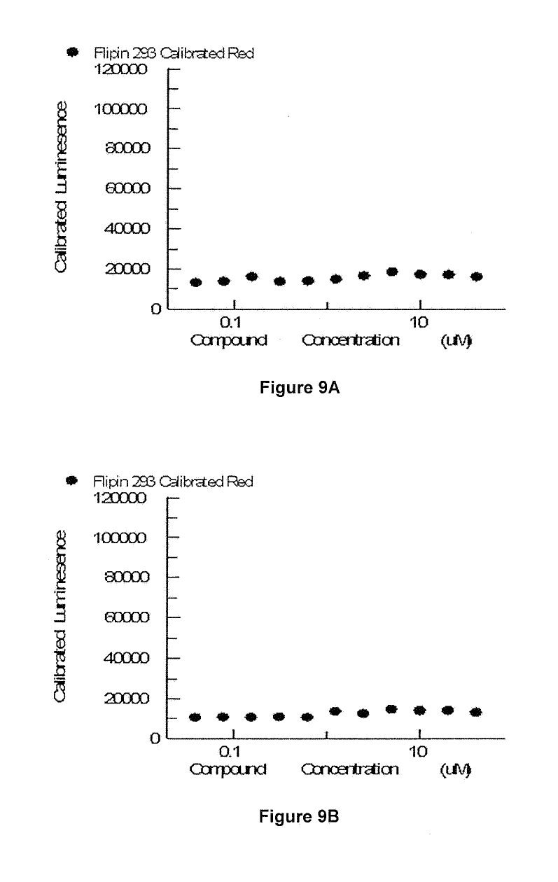

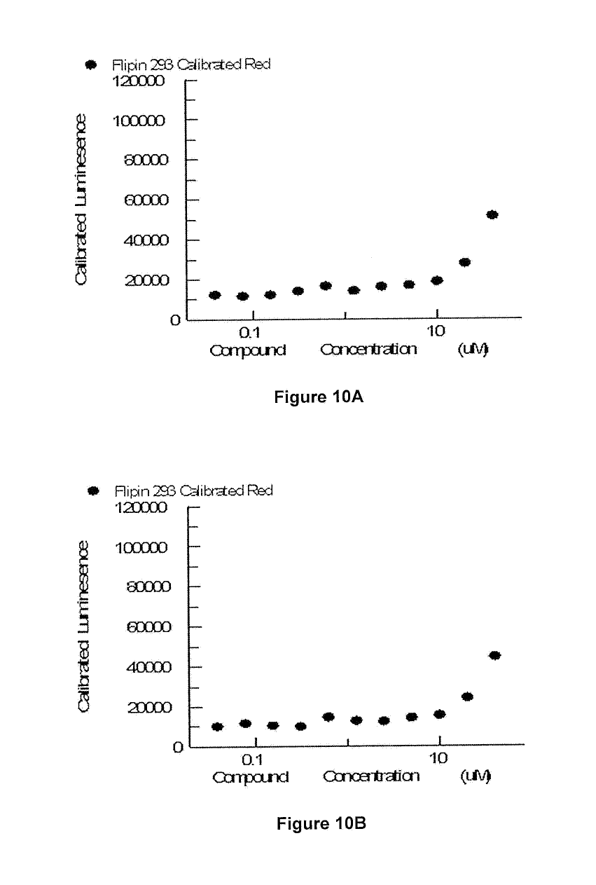

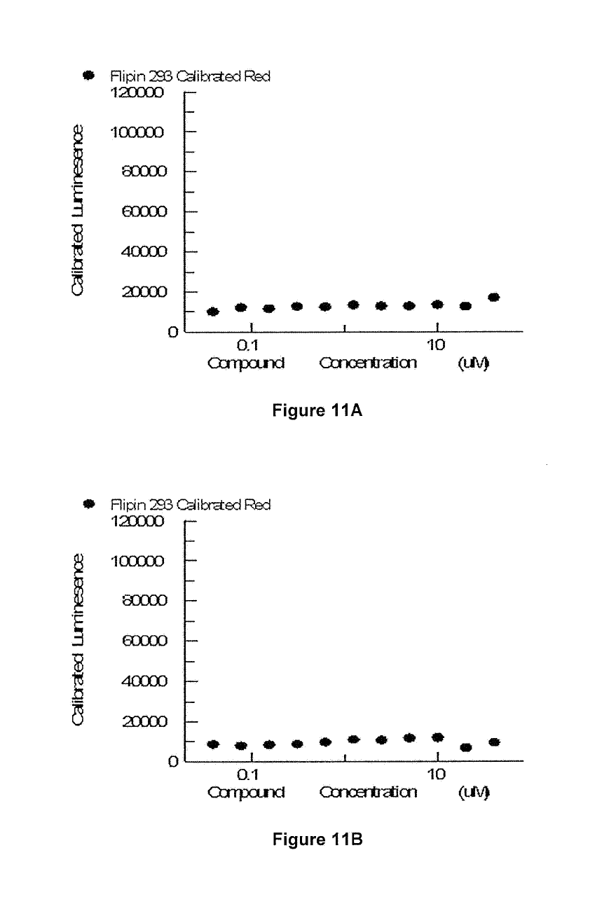

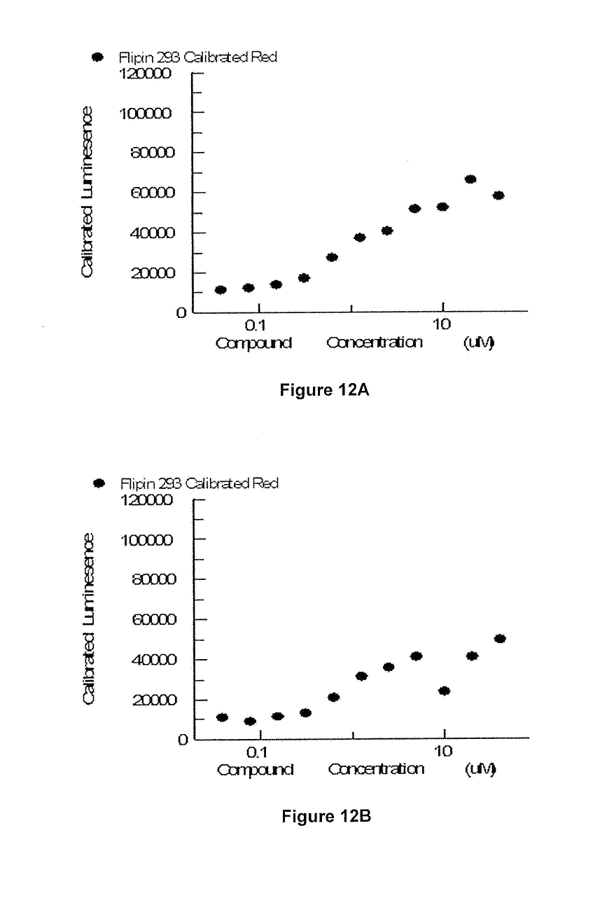

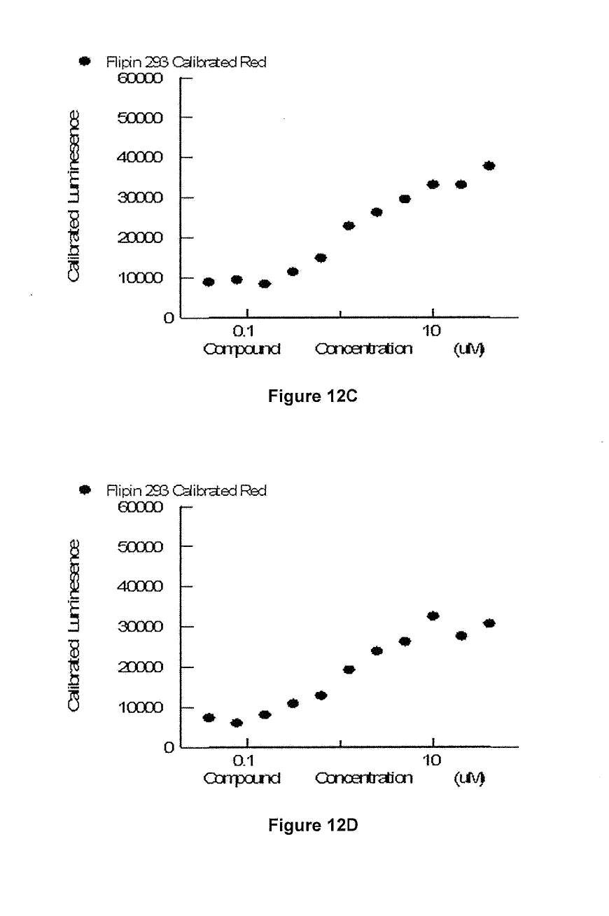

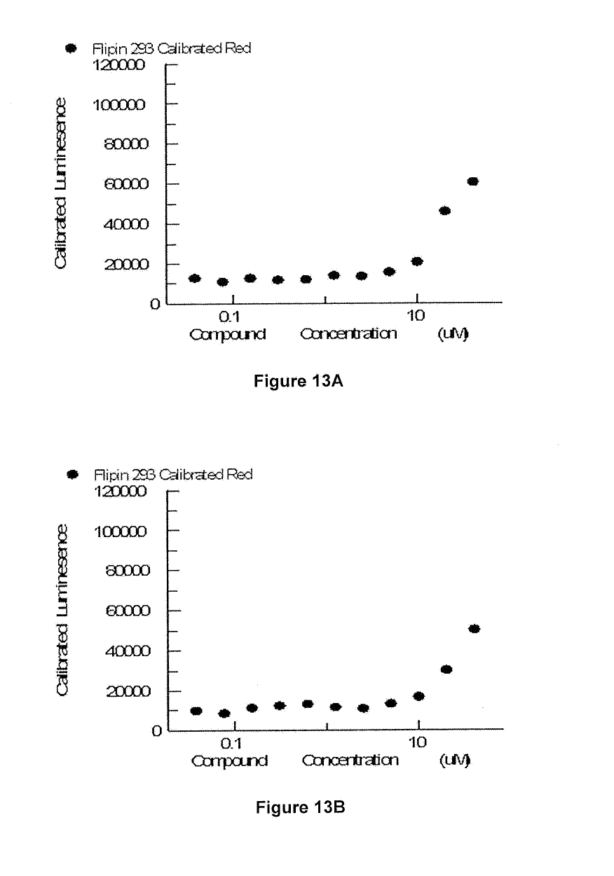

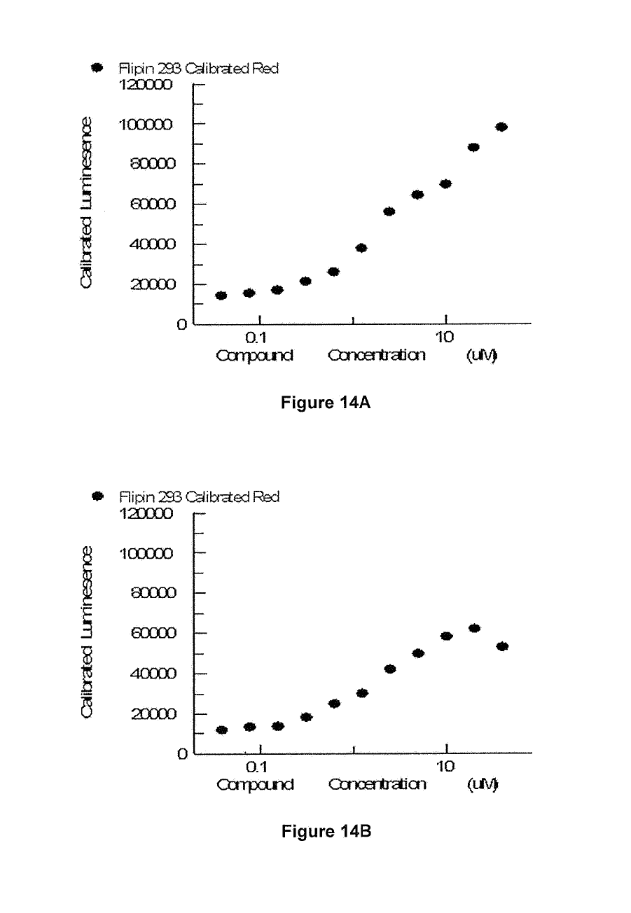

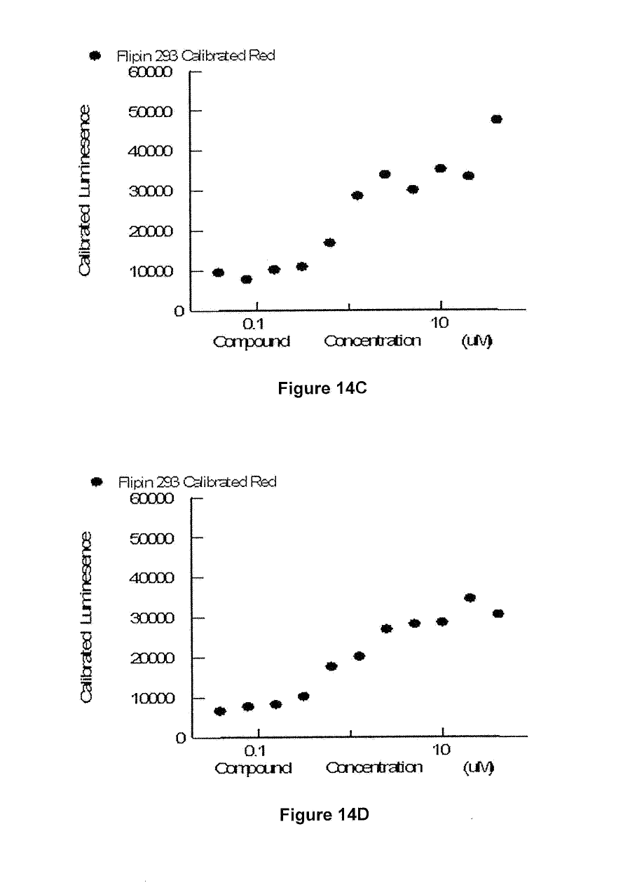

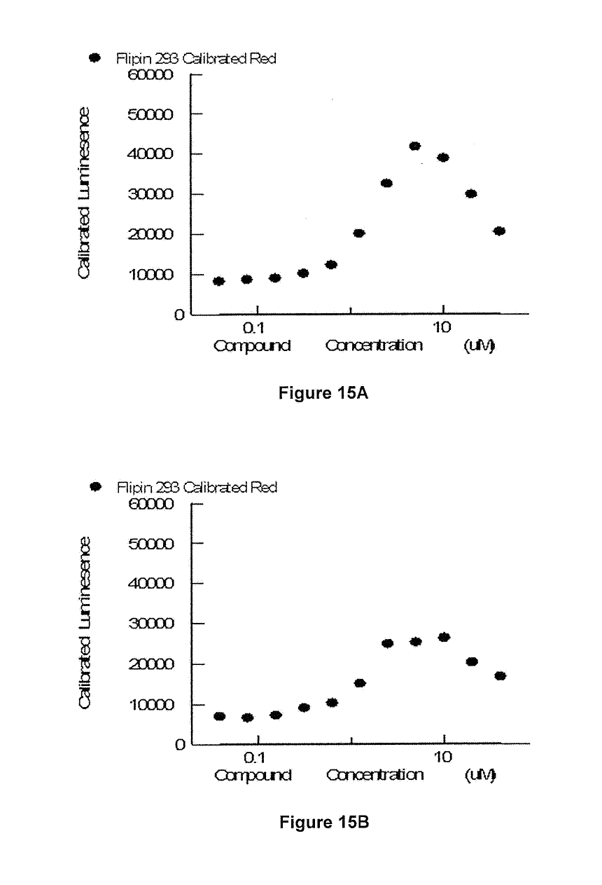

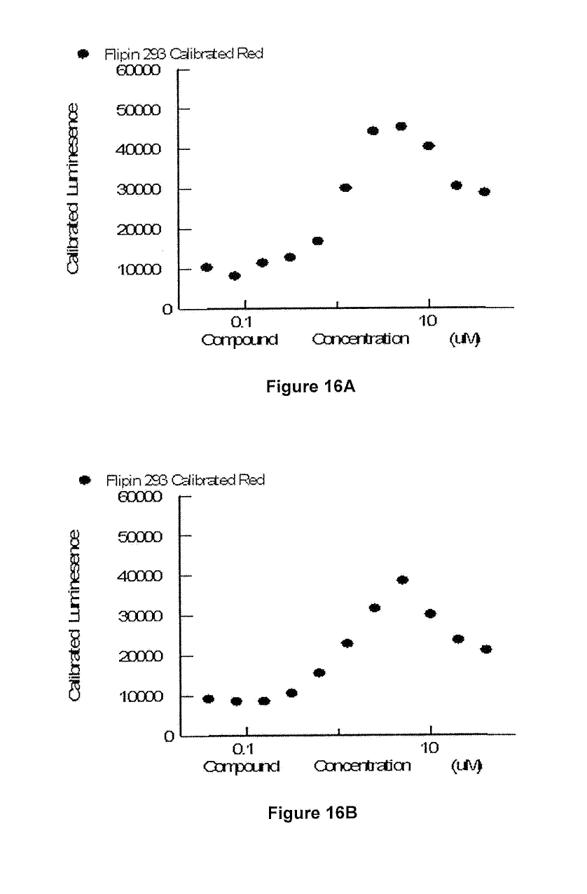

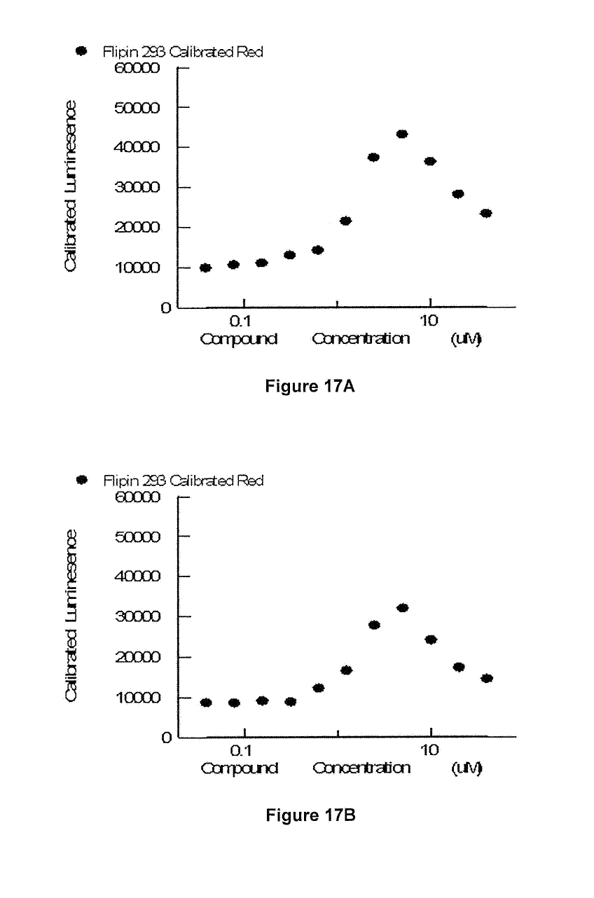

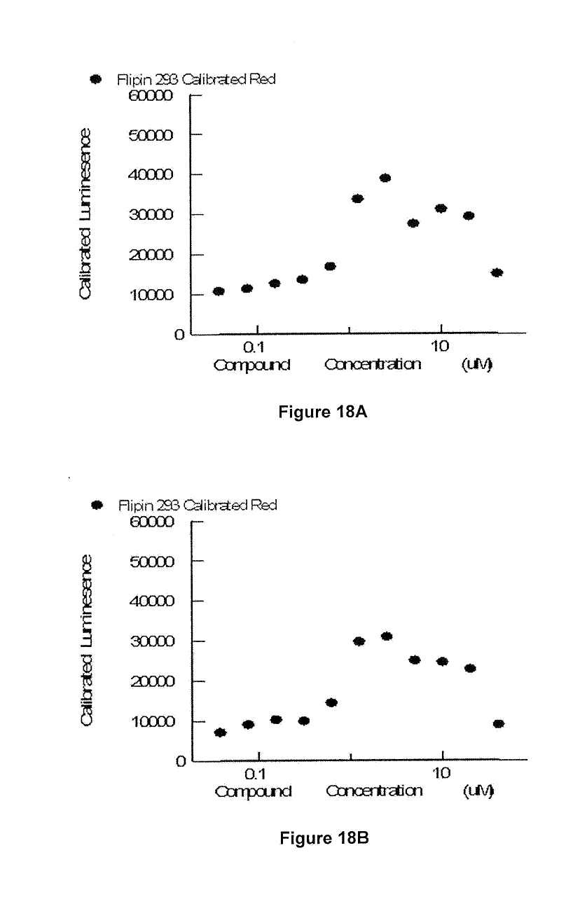

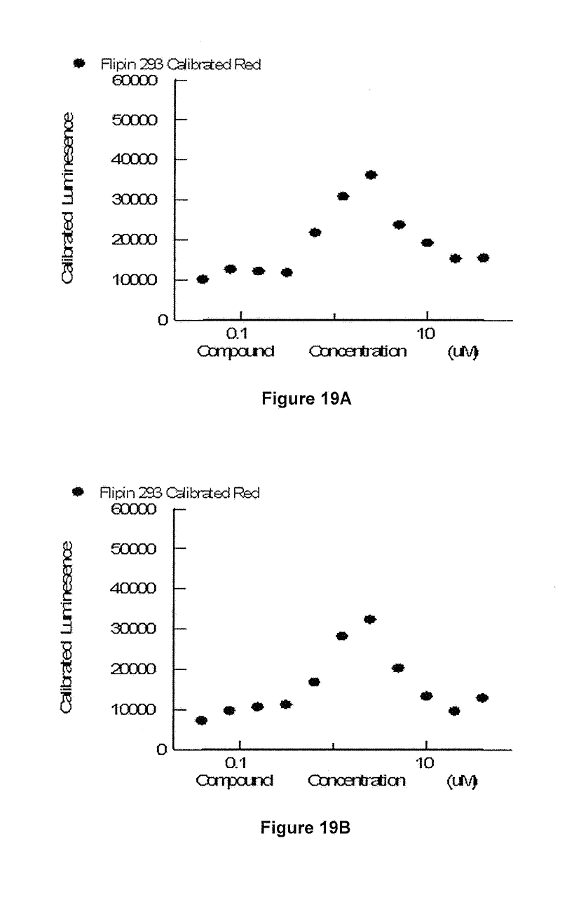

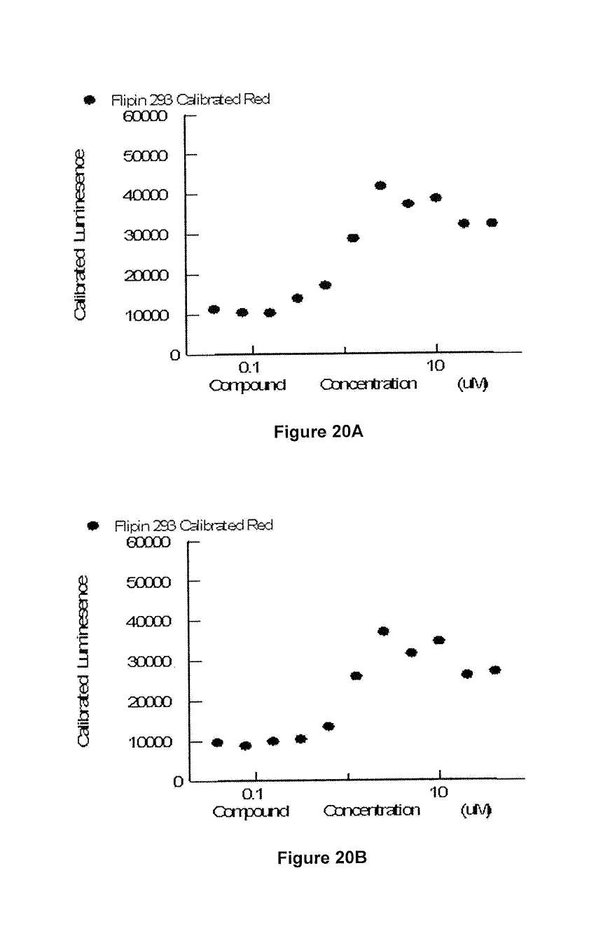

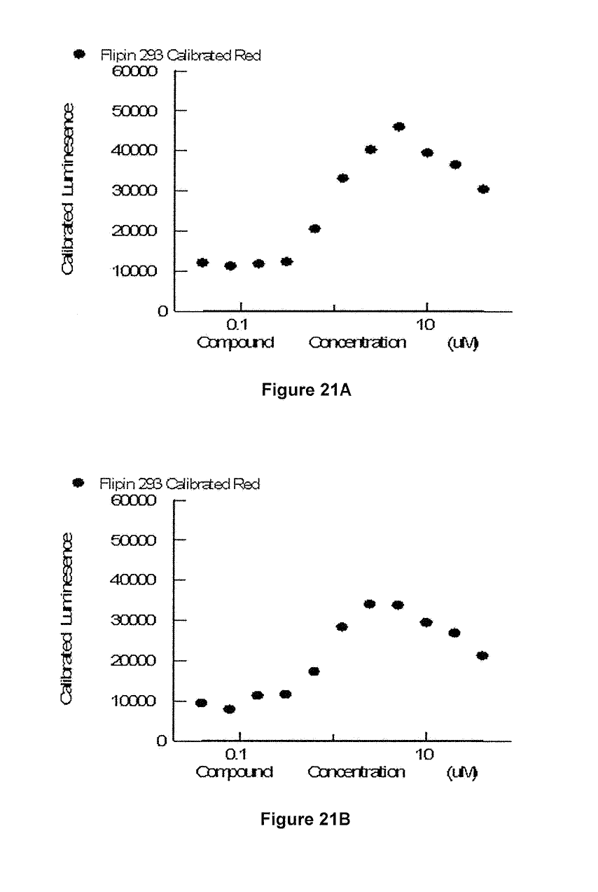

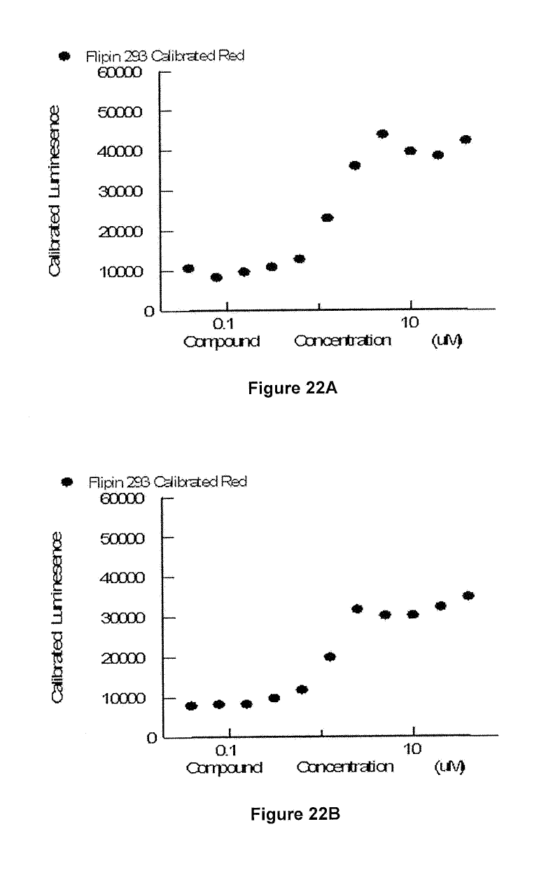

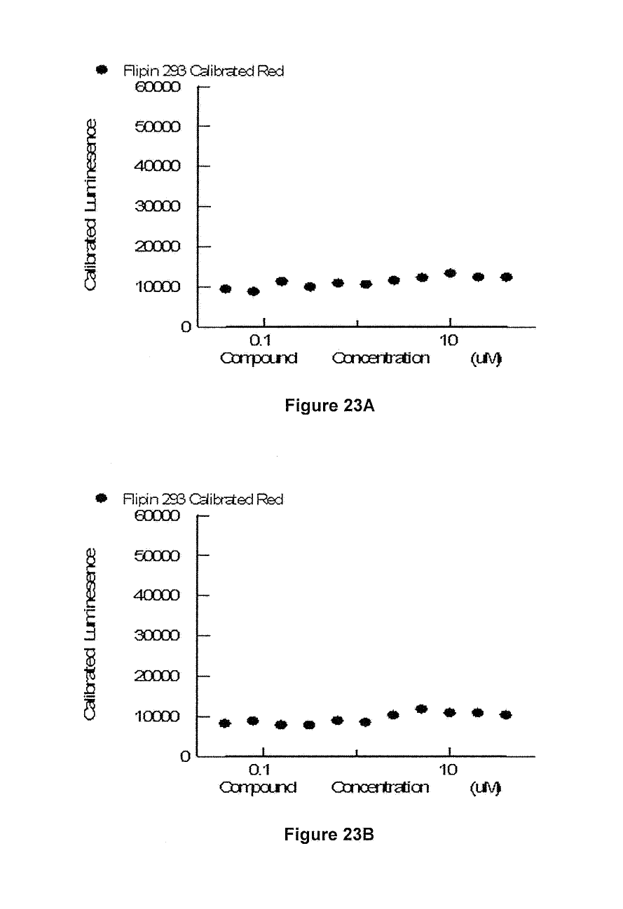

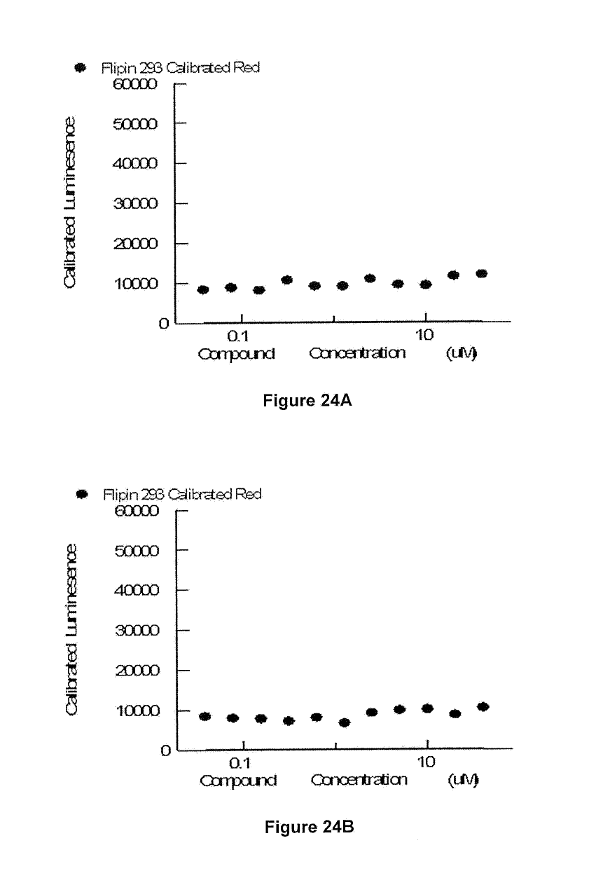

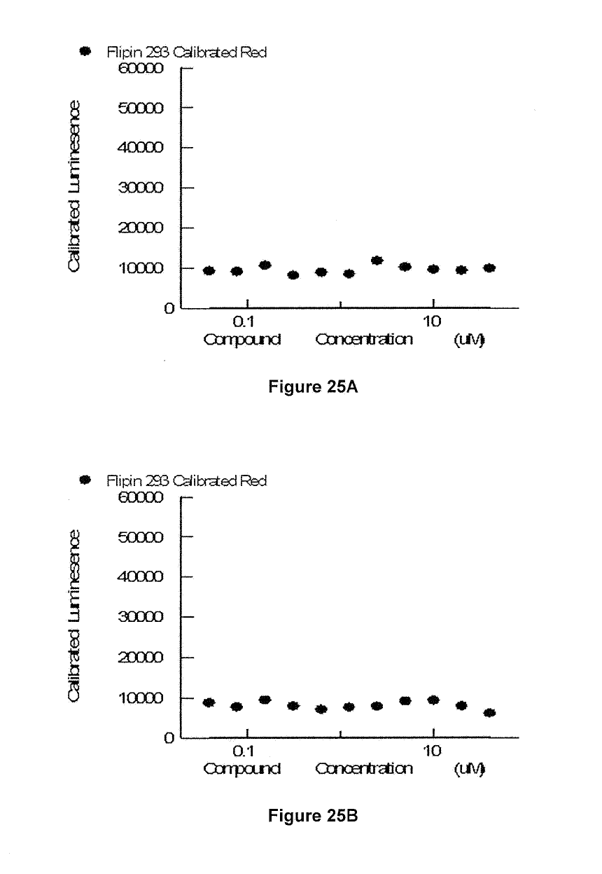

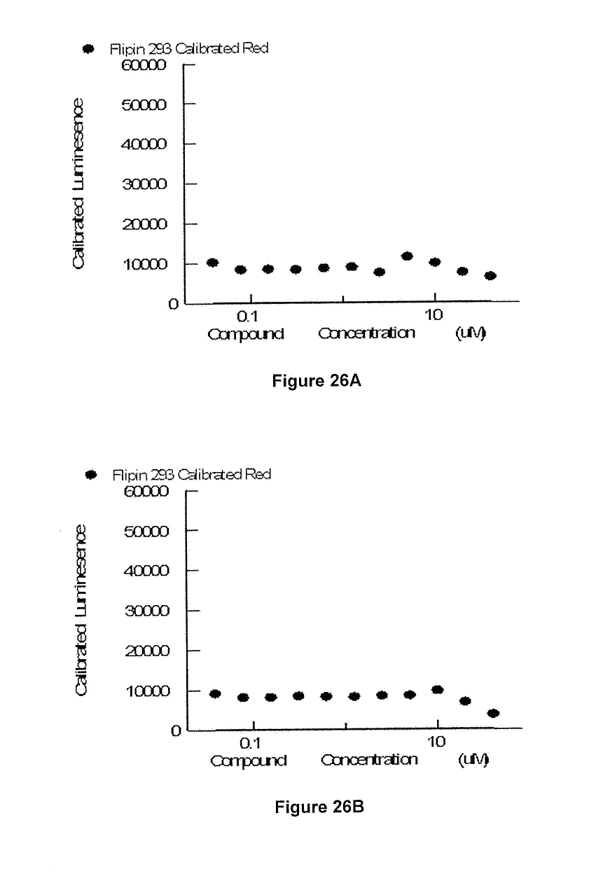

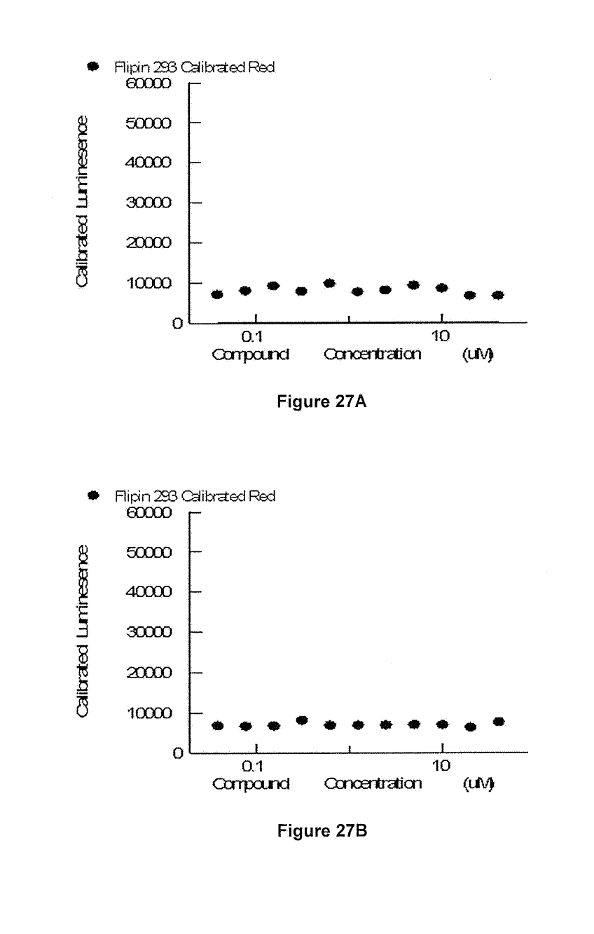

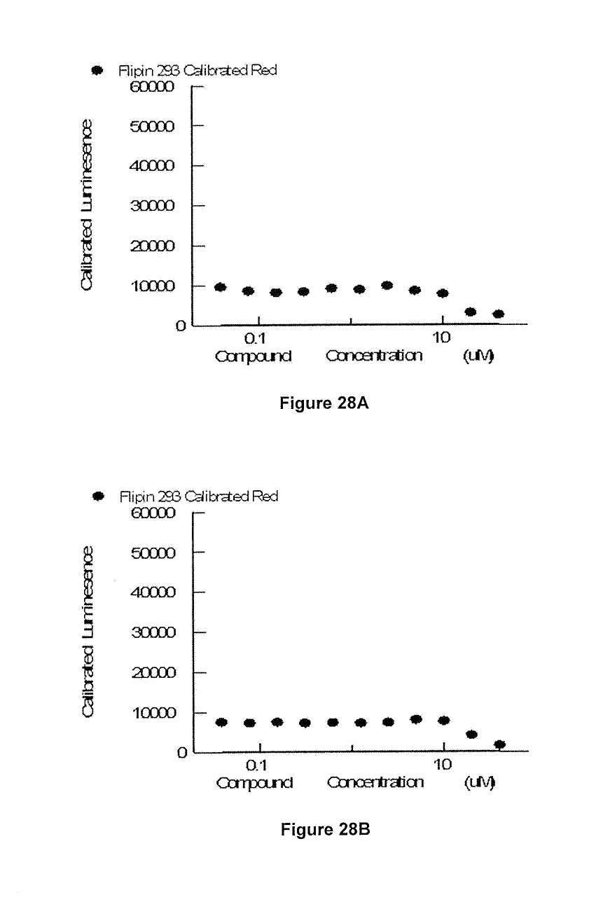

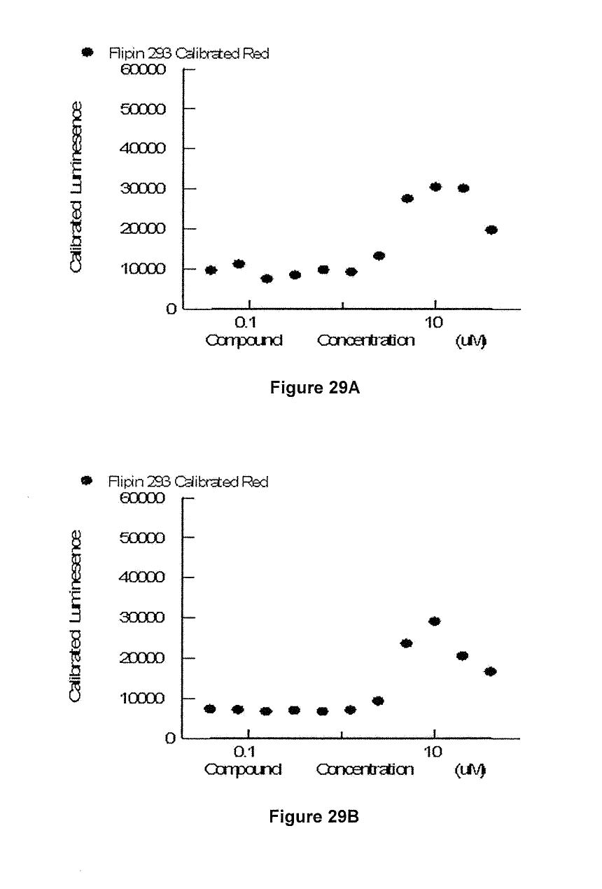

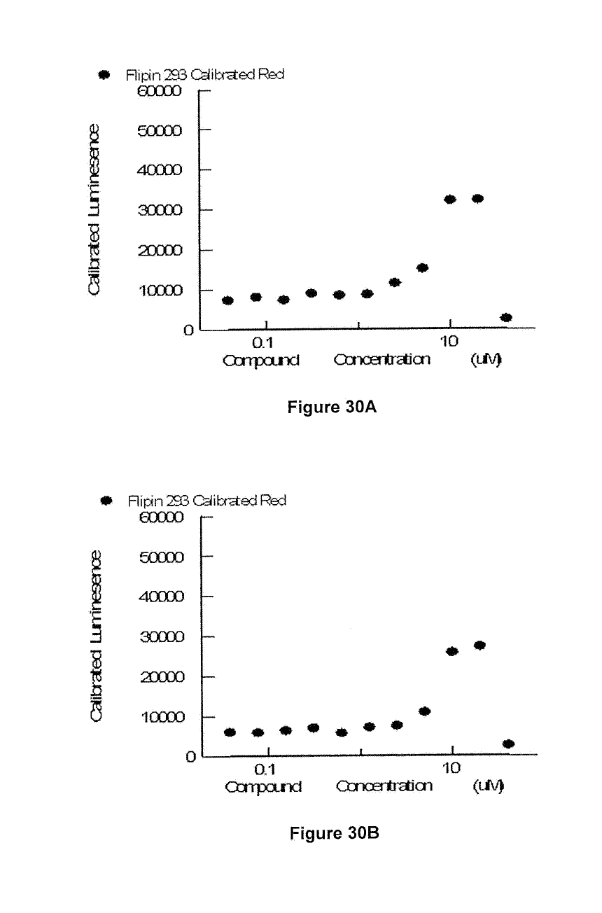

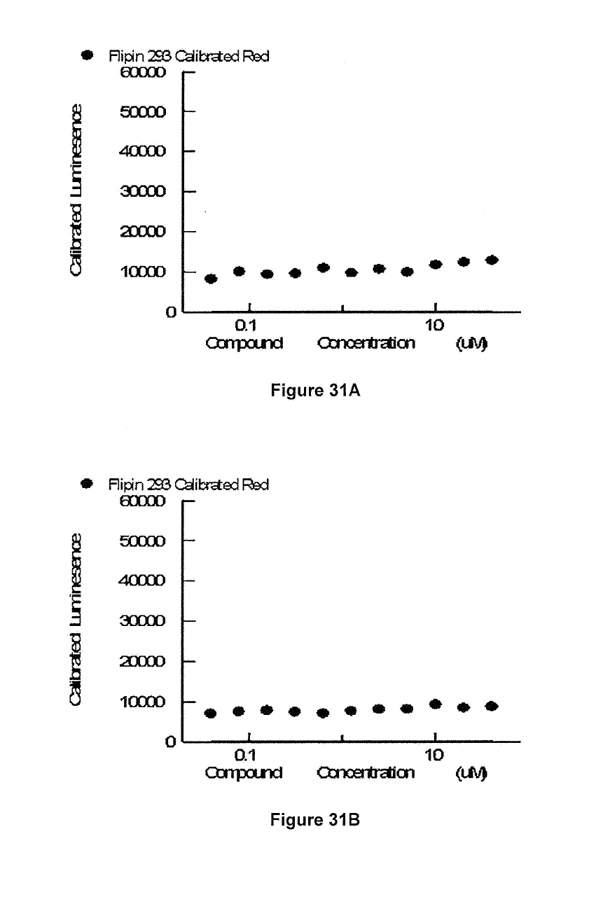

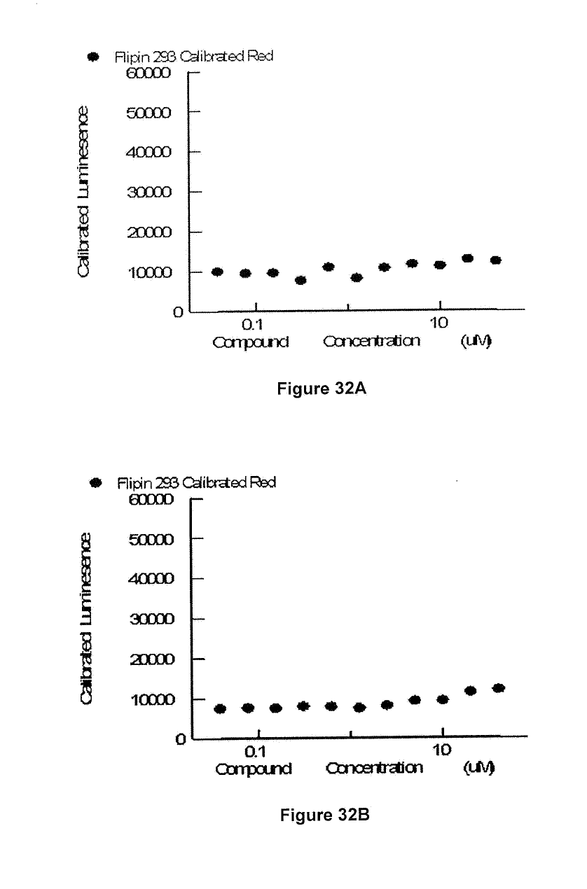

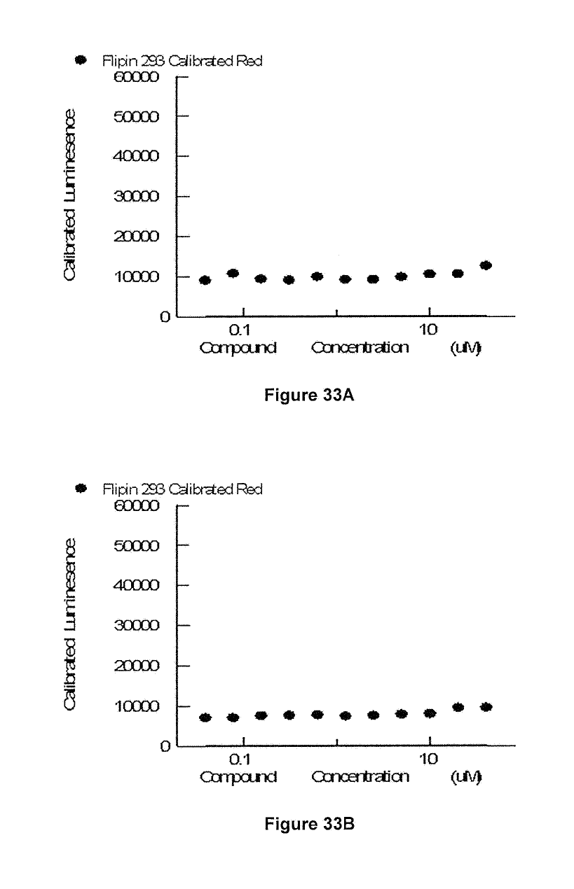

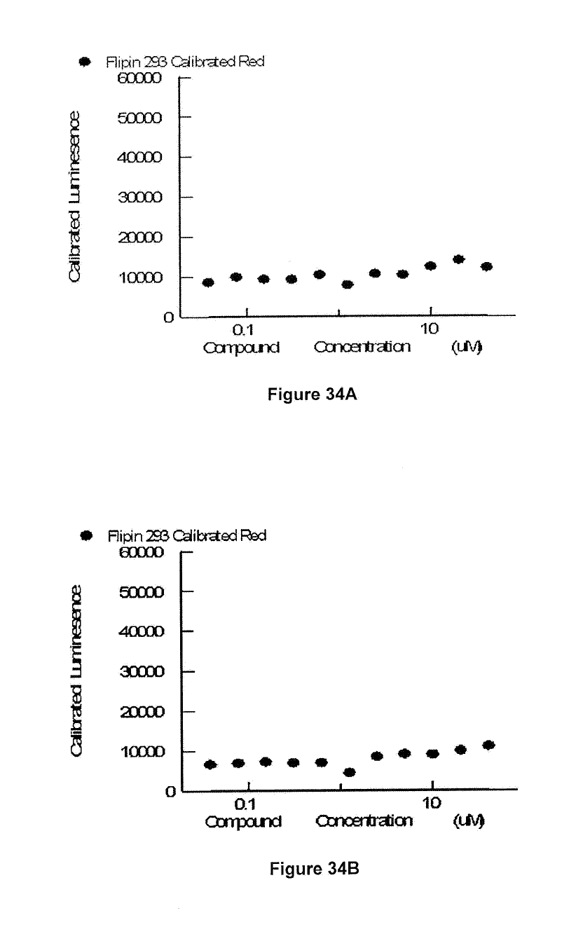

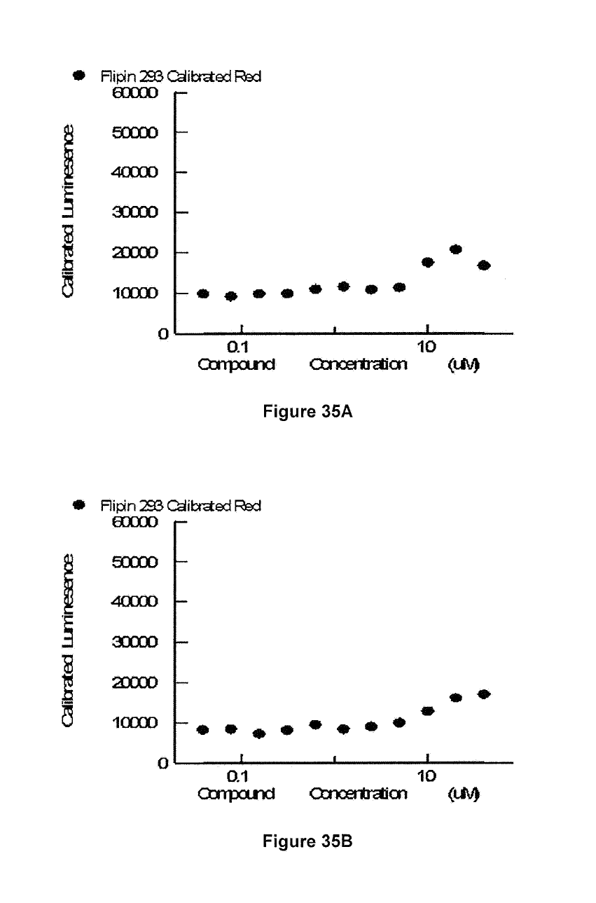

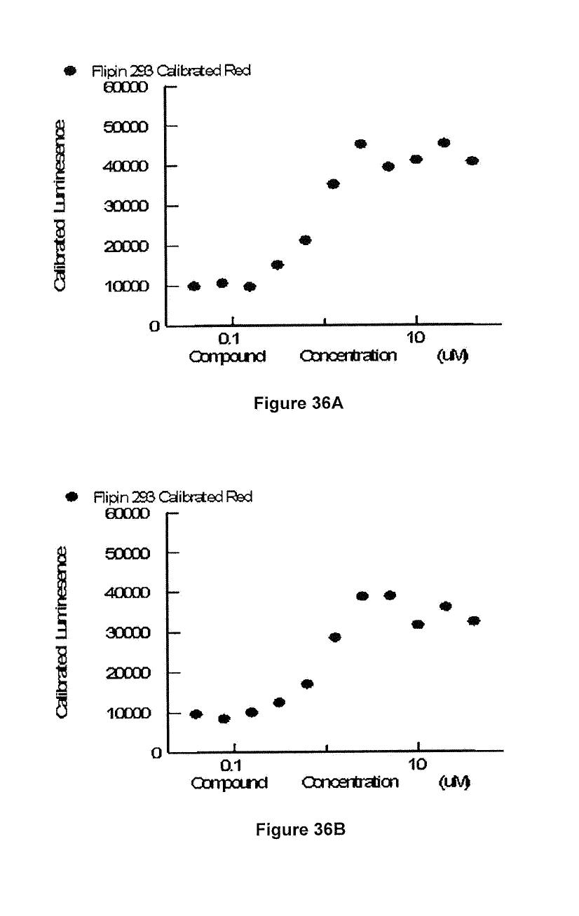

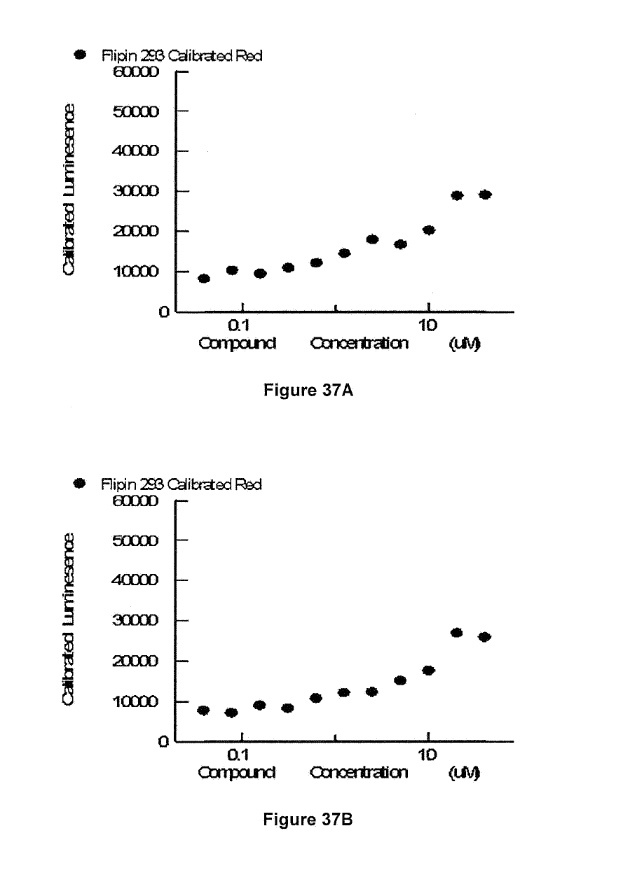

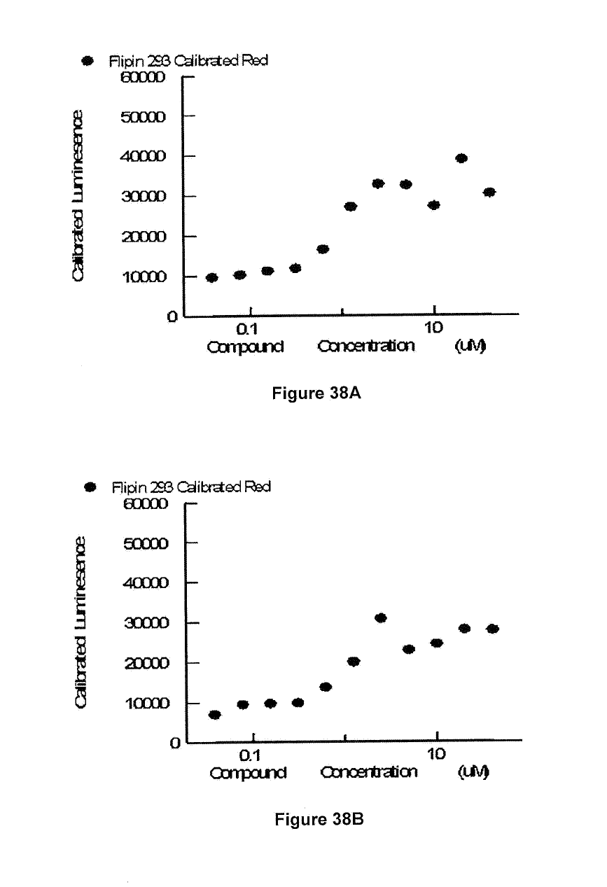

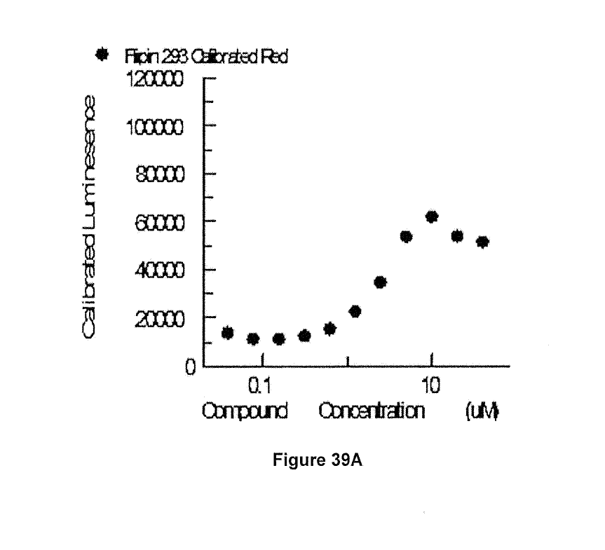

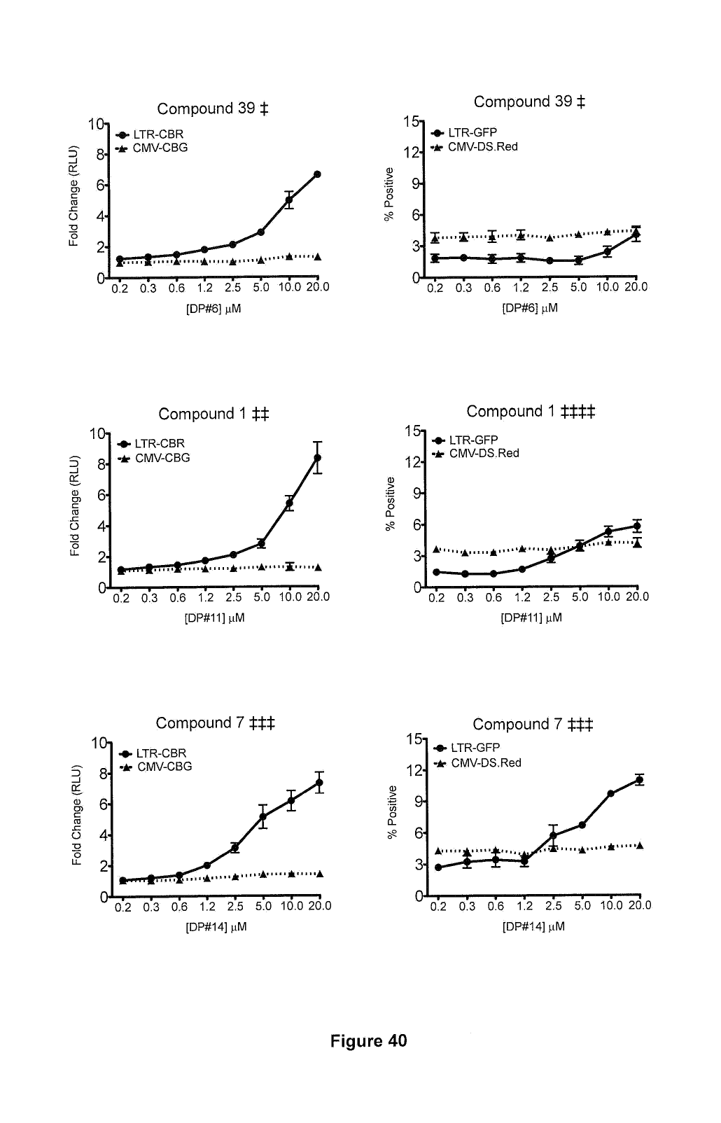

### DETAILED DESCRIPTION OF THE EMBODIMENTS

In one aspect, the present invention provides a compound of Formula (I):

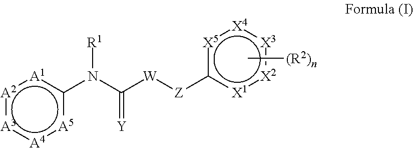

or a salt, solvate, or prodrug thereof

wherein

A¹, A², A³, A⁴ and A⁵ are independently selected from the group consisting of CR′, NR″, O and S, wherein A⁵ may or may not be present;

R′ is selected from the group consisting of H, C₁-C₄alkyl, O(C₁-C₄alkyl), CONR⁵R⁶, halo, CF₃, CF₂H and CN;

R″ is selected from H and C₁-C₄alkyl, wherein R″ may or may not be present;

R¹ is selected from H and C₁-C₄alkyl;

Y is selected from O and NH;

wherein when Y is NH and A⁵ is CH, optionally Y and A⁵ together form an imidazole ring so that the compound has the structure:

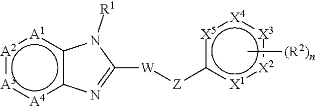

W is selected from the group consisting of C₁-C₄alkyl, NH, N(C₁-C₄alkyl) and O;

Z is selected from the group consisting of C₁-C₄alkyl, (CH₂)ₘO, (CH₂)ₘNH, (CH₂)ₘN(CH₃), and m is 0 or 1, wherein when W is O, m is 1;

alternatively W and Z together form an optionally substituted piperazine or piperidine ring so that the compound has the structure:

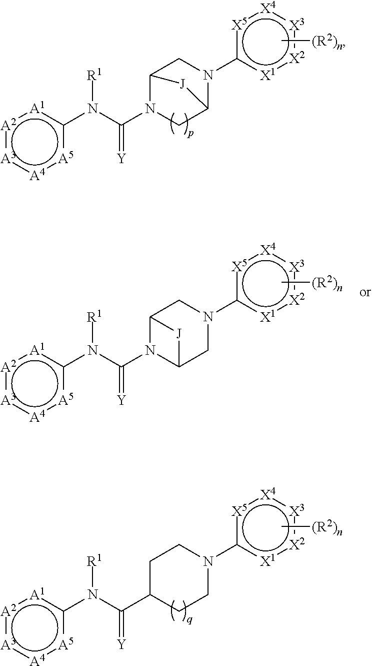

J is selected from CH₂ and (CH₂)₂, wherein J may or may not be present, p is 1 or 2, and q is 0 or 1;

X¹, X², X³, X⁴ and X⁵ are independently selected from the group consisting of CH, N, NH, O and S, wherein X⁵ may or may not be present;

each R² is independently selected from the group consisting of C₁-C₄alkyl, CN, CF₃, F, Cl, Br, hydroxyl, nitro, OR⁶, COR⁶, CO₂R⁶, CONR⁵R⁶, CONHSO₂R⁵, SO₂NHCOR⁵, CONR⁵OR⁶, C₁-C₄alkylNR⁵R⁶, C₁-C₄alkylOR⁶, NR⁵R⁶, NR⁵COR⁶, NR⁷CONR⁵R⁶ and NR⁵CO₂R⁶;

n is 0-3;

R⁵ and R⁶ are independently selected from the group consisting of H, C₁-C₄alkyl, C₃-C₁₀cycloalkyl, C₃-C₁₀heterocyclyl, C₆-C₁₀aryl, C₅-C₁₀heteroaryl, (C₁-C₄alkyl)C₆-C₁₀aryl and (C₁-C₄alkyl)C₅-C₁₀heteroaryl;

alternatively when R⁵ and R⁶ are bound to the same atom they form an optionally substituted C₃-C₁₀cycloalkyl or C₃-C₁₀heterocyclyl;

R⁷ is selected from H and CH₃.

In one embodiment, A⁵ is present, preferably A⁵ is CH. In a preferred embodiment, A⁵ is not present so that the compound has the structure:

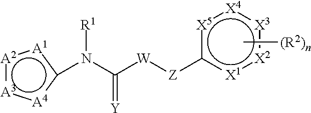

In one embodiment, the compound has the structure:

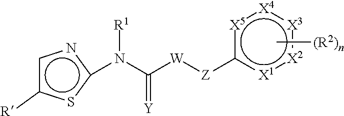

In one embodiment, the compound has the structure:

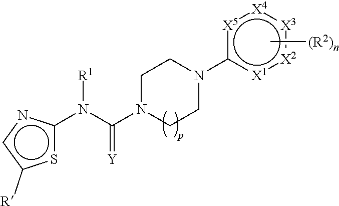

In one embodiment, the compound has the structure:

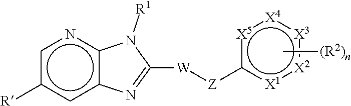

In one embodiment, A¹ is selected from CH and N, preferably A¹ is N.

In another embodiment, A² is selected from CH, N, N(CH₃), and O, preferably A² is CH.

In yet another embodiment, A³ is selected from CH, C(CH₃), C(CH₂CH₃), C(Br), C(Cl), C(CN), C(CF₃), and N(CH₃), preferably A³ is selected from C(CH₃), C(Br), C(Cl) and C(CN), more preferably A³ is C(CH₃).

In another embodiment, A⁴ is selected from S, O, CH, and NH, preferably A⁴ is S.

In a preferred embodiment, A¹, A², A³, A⁴ and A⁵ form a ring which does not include 2 heteroatoms adjacent to one another. In one embodiment, the ring does not include 2 nitrogen heteroatoms adjacent to one another. In another embodiment, the ring does not include 2 oxygen heteroatoms adjacent to one another. In yet another embodiment, the ring does not include a nitrogen heteroatom and an oxygen heteroatom adjacent to one another.

In a preferred embodiment, R¹ is H.

In another preferred embodiment, Y is O.

In another embodiment, W is C₁-C₄alkyl, preferably W is (CH₂)₂.

In another embodiment, Z is selected from C₁-C₄alkyl and (CH₂)ₘO, preferably Z is selected from CH₂, (CH₂)₂ and (CH₂)O, more preferably Z is (CH₂)O.

In another embodiment, X¹, X², X³, and X⁴ are each CH.

In one embodiment, X⁵ is present, preferably X⁵ is CH.

In one embodiment, each R² is independently selected from the group consisting of Br, Cl, CH₃, CF₃, and CN, preferably each R² is independently selected from Br and Cl.

In a preferred embodiment, n is 2.

In one embodiment, R² is located at positions 3 and 4, so that the compound is of the form:

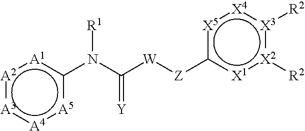

In a preferred embodiment, the compound is selected from the group consisting of:

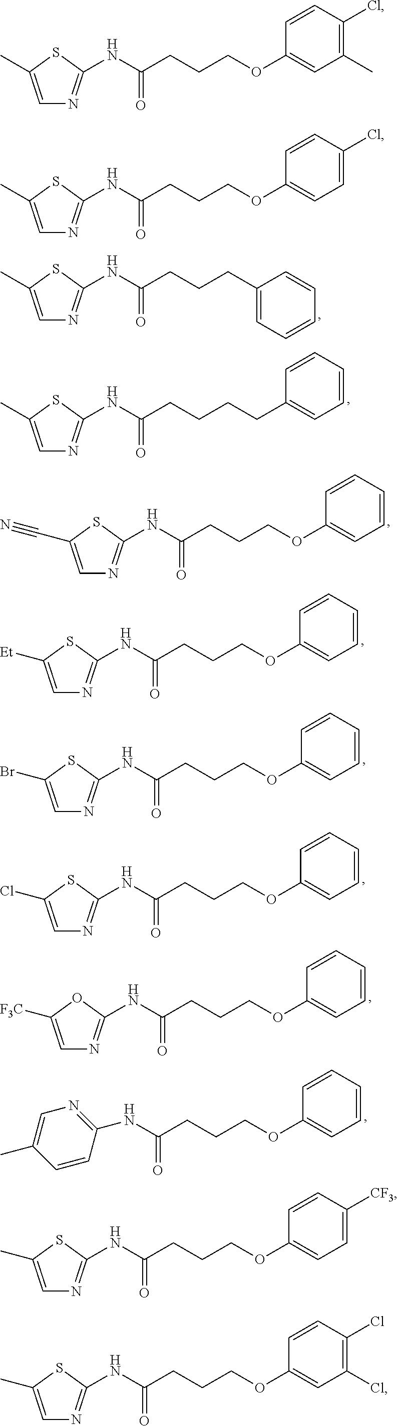

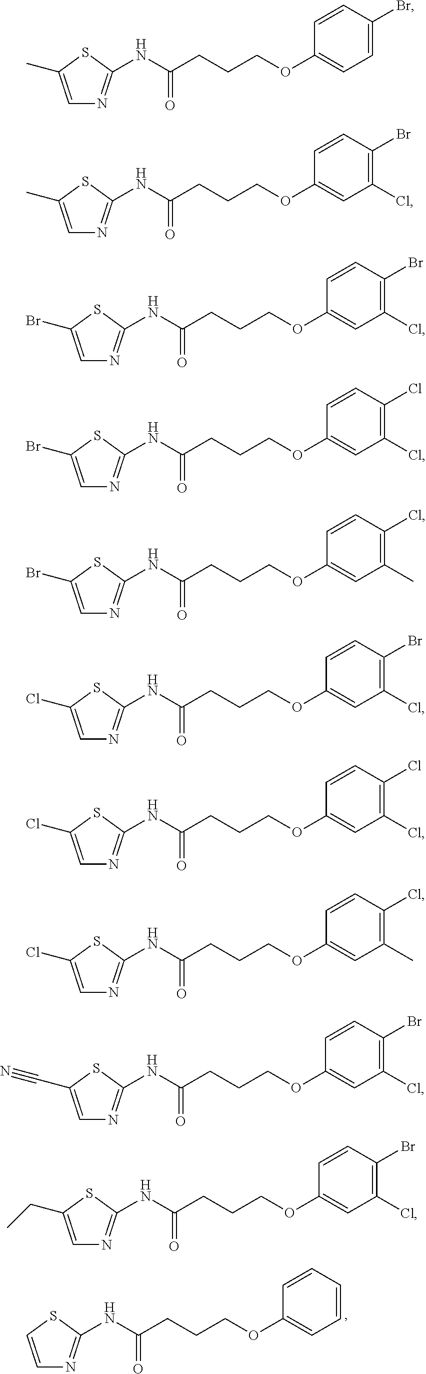

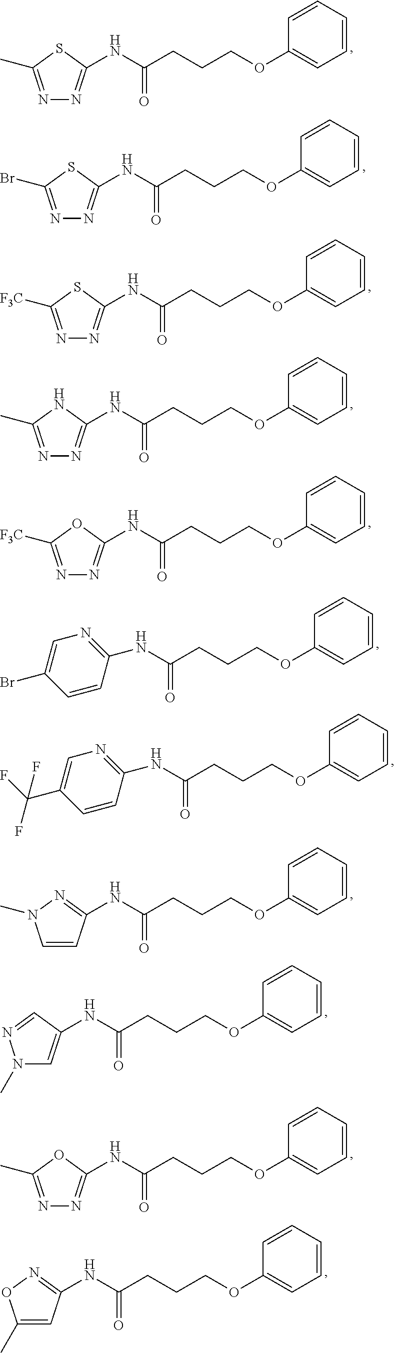

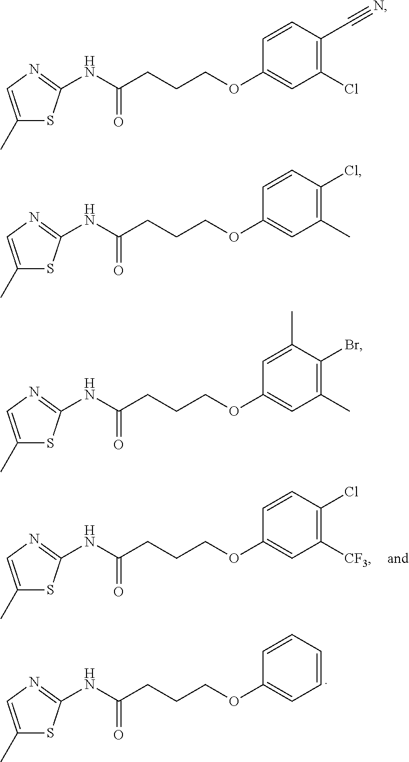 [SMILES: Cc1cnc(NC(=O)CCCOc2ccc(C#N)c(Cl)c2)s1 ; Cc1cnc(NC(=O)CCCOc2ccc(Cl)c(C)c2)s1 ; Cc1cnc(NC(=O)CCCOc2cc(C)c(Br)c(C)c2)s1 ; Cc1cnc(NC(=O)CCCOc2ccc(Cl)c(C(F)(F)F)c2)s1 ; Cc1cnc(NC(=O)CCCOc2ccccc2)s1]

In a particularly preferred embodiment, the compound is selected from the group consisting of:

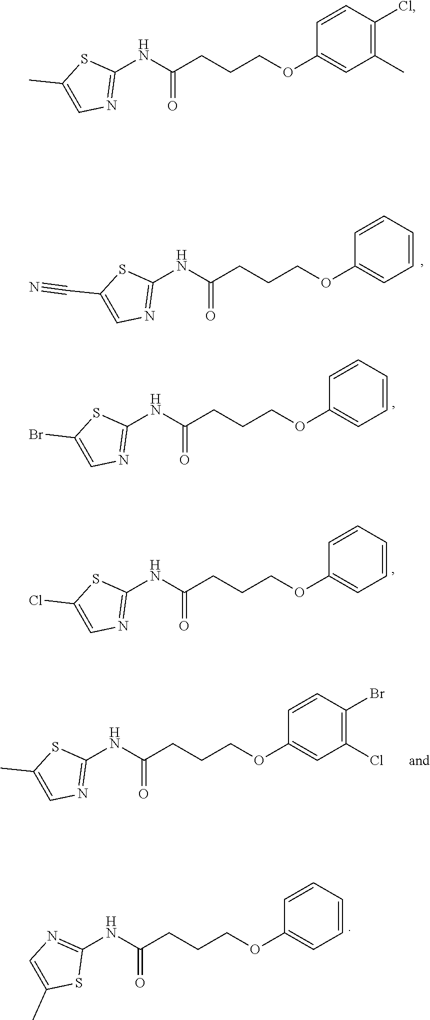

Even more preferred is the compound:

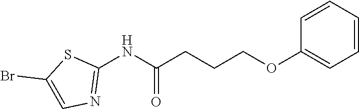 [SMILES: O=C(CCCOc1ccccc1)Nc1ncc(Br)s1]

In one embodiment, the compound is selected from the group consisting of:

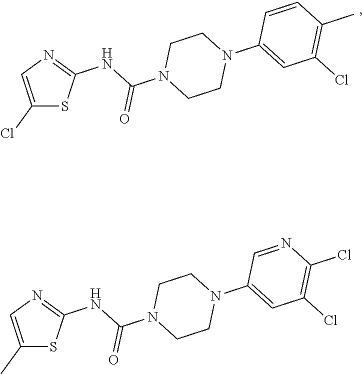

In another embodiment, the compound is selected from the group consisting of compounds 40 to 87.

In another embodiment, the compound is selected from the group consisting of compounds 42 to 87.

In one embodiment, the compound is not selected from the group consisting of:

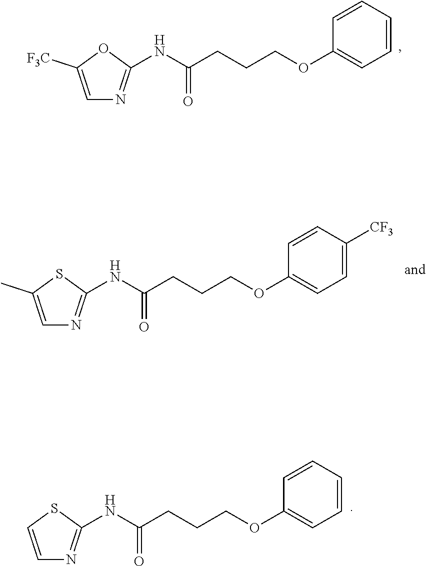 [SMILES: O=C(CCCOc1ccccc1)Nc1ncc(C(F)(F)F)o1 ; Cc1cnc(NC(=O)CCCOc2ccc(C(F)(F)F)cc2)s1 ; O=C(CCCOc1ccccc1)Nc1nccs1]

In one embodiment, the compound is not selected from the group consisting of:

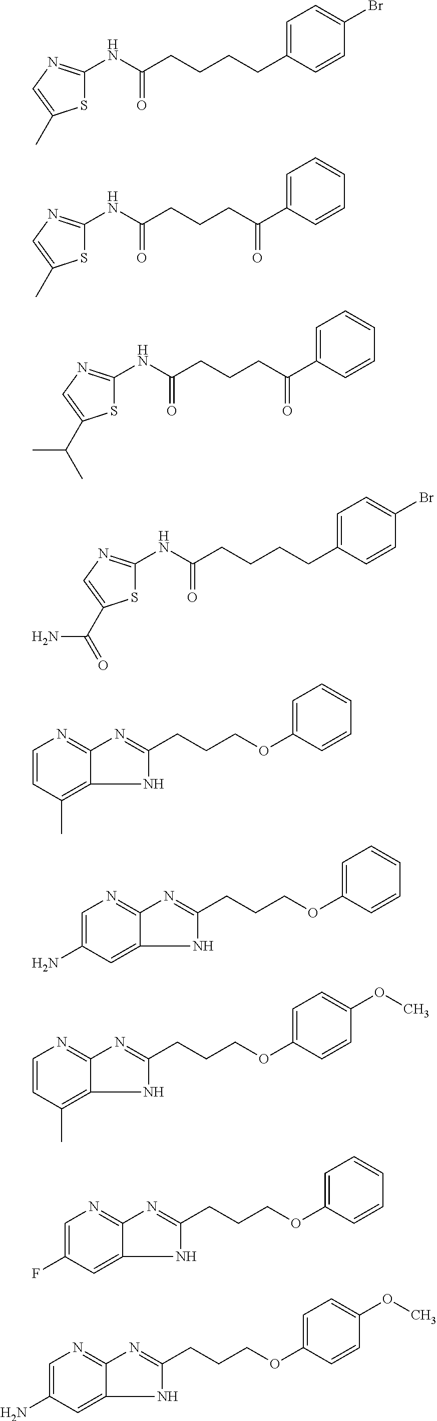

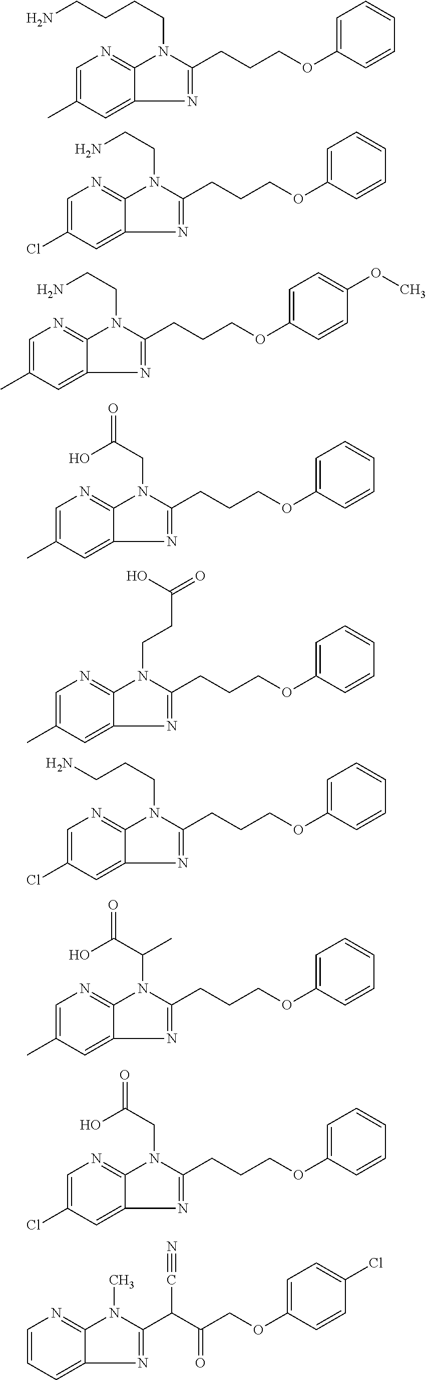

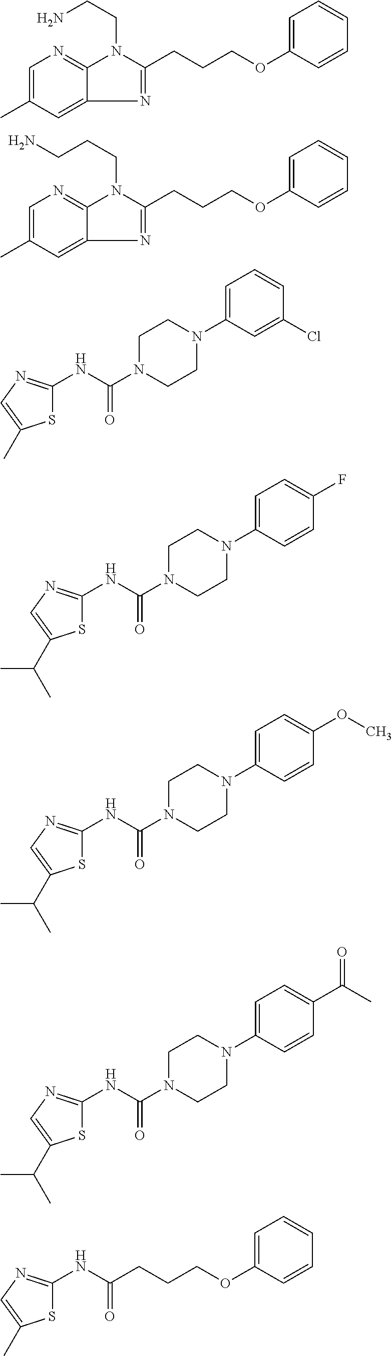

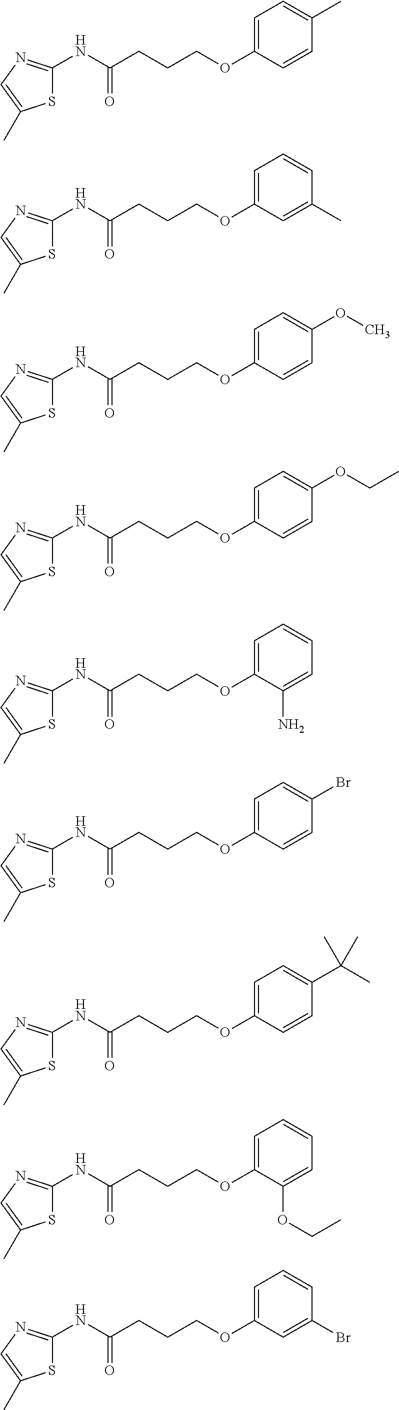

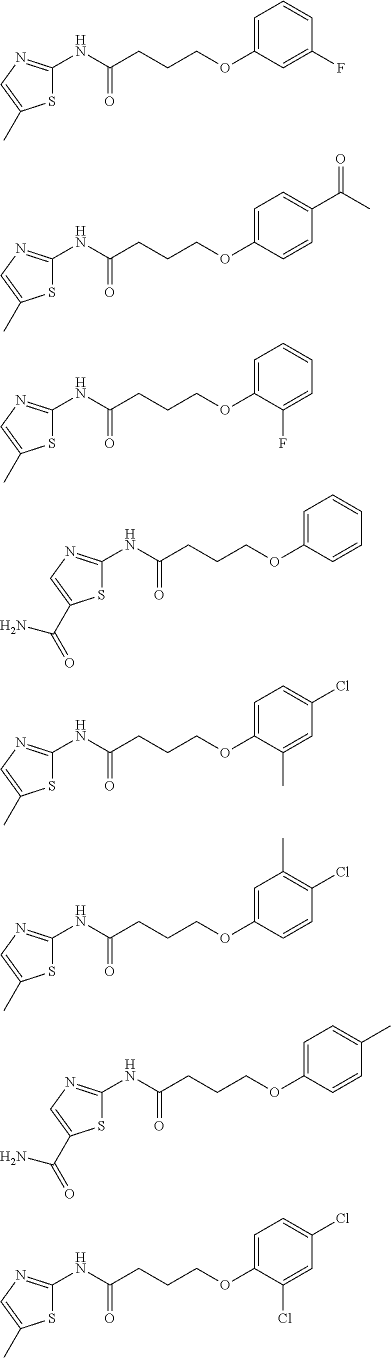

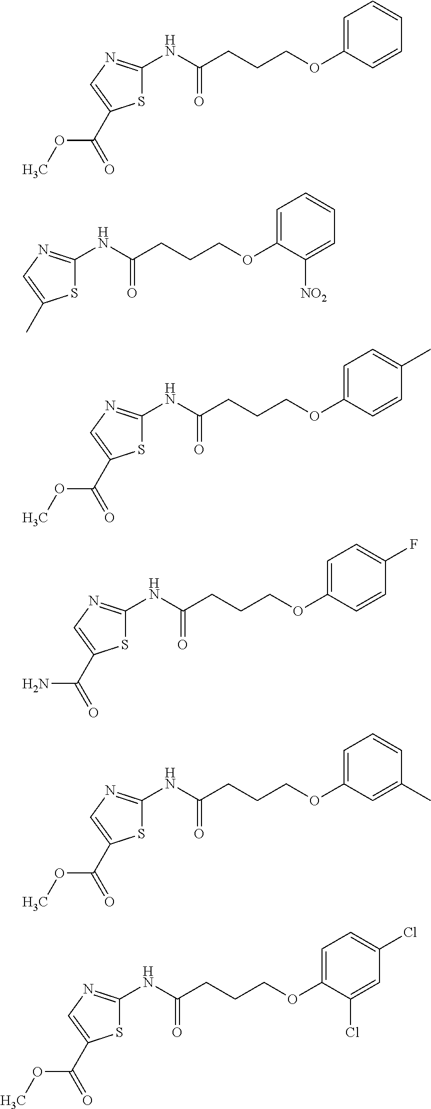

Compounds are generally described herein using standard nomenclature. For compounds having asymmetric centres, it will be understood that, unless otherwise specified, all of the optical isomers and mixtures thereof are encompassed. Compounds with two or more asymmetric elements can also be present as mixtures of diastereomers. In addition, compounds with carbon-carbon double bonds may occur in Z and E forms, with all isomeric forms of the compounds being included in the present invention unless otherwise specified. Where a compound exists in various tautomeric forms, a recited compound is not limited to any one specific tautomer, but rather is intended to encompass all tautomeric forms. Recited compounds are further intended to encompass compounds in which one or more atoms are replaced with an isotope, i.e., an atom having the same atomic number but a different mass number. By way of general example, and without limitation, isotopes of hydrogen include tritium and deuterium and isotopes of carbon include ¹¹C, ¹³C, and ¹⁴C.

Compounds according to the formula provided herein, which have one or more stereogenic centres, have an enantiomeric excess of at least 50%. For example, such compounds may have an enantiomeric excess of at least 60%, 70%, 80%, 85%, 90%, 95%, or 98%. Some embodiments of the compounds have an enantiomeric excess of at least 99%. It will be apparent that single enantiomers (optically active forms) can be obtained by asymmetric synthesis, synthesis from optically pure precursors, biosynthesis or by resolution of the racemates, for example, enzymatic resolution or resolution by conventional methods such as crystallization in the presence of a resolving agent, or chromatography, using, for example, a chiral HPLC column.

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₃ alkyl”, “C₁-C₄ alkyl” and “C₁-C₆ alkyl” refer to an alkyl group, as defined above, containing at least 1, and at most 3, 4 or 6 carbon atoms respectively, or any range in between (e.g. alkyl groups containing 2-5 carbon atoms are also within the range of C₁-C₆). Where the term “C₀-C₂ alkyl” is used, there may be no alkyl group, or an alkyl group containing 1 or 2 carbon atoms.

As an example of substituted alkyls, the term —(C₁-C₄ alkyl)N(C₁-C₄ alkyl)₂ includes —CH₂N(CH₃)₂, —(CH₂)₂N(CH₃)₂, —CH₂N(CH₂CH₃)₂, —CH₂N(iPr)(CH₃), and the like.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is bromo or chloro.

As used herein, the term “cycloalkyl” refers to a non-aromatic cyclic hydrocarbon ring. In a like manner the term “C₃-C₇ cycloalkyl” refers to a non-aromatic cyclic hydrocarbon ring having from three to seven carbon atoms, or any range in between. For example, the C₃-C₇ cycloalkyl group would also include cycloalkyl groups containing 4 to 6 carbon atoms. The alkyl group is as defined above, and may be substituted. Exemplary “C₃-C₇ cycloalkyl” groups useful in the present invention include, but are not limited to, cyclopropyl, cyclobutyl, cyclopentyl, cyclohexyl and cycloheptyl.

As used herein, the terms “heterocyclic” or “heterocyclyl” refer to a nonaromatic heterocyclic ring, being saturated or having one or more degrees of unsaturation, containing one or more heteroatom substitution selected from S, S(O), S(O)₂, O, or N.

The heterocyclyl group may be attached through any atom of its structure, including a heteroatom. The term “C₃-C₇ heterocyclyl” refers to a non-aromatic cyclic hydrocarbon ring having from three to seven carbon atoms containing one or more heteroatom substitutions as referred to herein. The heterocyclic moiety may be substituted, multiple degrees of substitution being allowed. The term “C₃-C₇ heterocyclyl” also includes heterocyclyl groups containing C₄-C₅, C₅-C₇, C₆-C₇, C₄-C₇, C₄-C₆ and C₅-C₆ carbon atoms. Preferably, the heterocyclic ring contains four to six carbon atoms and one or two heteroatoms. More preferably, the heterocyclic ring contains five carbon atoms and one heteroatom, or four carbon atoms and two heteroatom substitutions, or four carbon atoms and one heteroatom, or four carbon atoms and two heteroatom substitutions. Such a ring may be optionally fused to one or more other “heterocyclic” ring(s) or cycloalkyl ring(s). Examples of “heterocyclic” moieties include, but are not limited to, tetrahydrofuran, pyran, oxetane, 1,4-dioxane, 1,3-dioxane, piperidine, piperazine, N-methylpiperazinyl, 2,4-piperazinedione, pyrrolidine, imidazolidine, pyrazolidine, morpholine, thiomorpholine, tetrahydrothiopyran, tetrahydrothiophene, and the like.

Cycloalkyl and heterocyclyl groups may be substituted with any suitable substituent as described below.

As an example of substituted heterocyclic groups, the term “(C₀-C₄ alkyl)C₃-C₇ heterocyclyl” includes heterocyclyl groups containing either no alkyl group as a linker between the compound and the heterocycle, or an alkyl group containing 1, 2, 3 or 4 carbon atoms as a linker between the compound and the heterocycle (eg. heterocycle, —CH₂-heterocycle or —CH₂CH₂-heterocycle). The alkyl linker can bind to any atom of the heterocyclyl group, including a heteroatom. Any of these heterocycles may be further substituted.

Substituted cycloalkyl and heterocyclyl groups may be substituted with any suitable substituent as described below.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Examples of “aryl” groups include, but are not limited to, phenyl, 2-naphthyl, 1-naphthyl, biphenyl, as well as substituted derivatives thereof. Preferred aryl groups include arylamino, aralkyl, aralkoxy, heteroaryl groups.

As used herein, the term “heteroaryl” refers to a monocyclic five, six or seven membered aromatic ring, or to a fused bicyclic or tricyclic aromatic ring system comprising at least one monocyclic five, six or seven membered aromatic ring. These heteroaryl rings contain one or more nitrogen, sulfur, and/or oxygen heteroatoms, where N-oxides and sulfur oxides and dioxides are permissible heteroatom substitutions and may be optionally substituted with up to three members. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, benzimidazolyl, and substituted versions thereof.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, i.e., a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable substituents for a particular functional group will be apparent to those skilled in the art.

Examples of substituents include but are not limited to:

C₁-C₆ alkyl, C₁-C₆ haloalkyl, C₁-C₀₆ haloalkoxy, C₁-C₆ hydroxyalkyl, C₁-C₀₆ hydroxyalkoxy, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halogen, ureido or C₁-C₆ perfluoroalkyl. In one embodiment, cyclic or heterocyclic substituents may form a spiro substituent with a carbon in the moiety from which the cyclic or heterocyclic group is substituted.

Any of these groups may be further substituted by any of the above-mentioned groups, where appropriate. For example, alkylamino, or dialkylamino, C₁-C₆ alkoxy, etc.

In certain embodiments the present invention provides compounds of Formula (I) wherein a combination of two or more of the preferred embodiments described herein are provided.

In a further aspect of the invention, there is provided novel compounds of Formula (I).

In yet another aspect, there is provided a composition comprising a compound according to Formula (I) or a salt, solvate, or prodrug thereof, and a pharmaceutically acceptable excipient.

In another aspect, there is provided a method for activating HIV expression in latently infected cells in a subject in need thereof, the method comprising administering an effective amount of a compound or a salt, solvate, or prodrug thereof of Formula (I) to the subject.

In another aspect, there is provided a method for activating HIV expression in latently infected cells in a subject in need thereof, the method comprising administering an effective amount of a composition comprising a compound or a salt, solvate, or prodrug thereof of Formula (I) to the subject.

In another aspect, there is provided a method for treating HIV infection in a subject in need thereof, the method comprising administering an effective amount of a compound or a salt, solvate, or prodrug thereof of Formula (I) in combination with a therapeutically effective amount of one or more anti-HIV viral therapy compounds to the subject.

In another aspect, there is provided a method for treating HIV infection in a subject in need thereof, the method comprising administering an effective amount of a composition comprising a compound or a salt, solvate, or prodrug thereof of Formula (I) in combination with a therapeutically effective amount of one or more anti-HIV viral therapy compounds to the subject.

In another aspect, there is provided use of a compound of Formula (I) or a salt, solvate, or prodrug thereof for activating HIV expression in latently infected cells in a subject in need thereof.

In another aspect, there is provided use of a composition comprising a compound of Formula (I) or a salt, solvate, or prodrug thereof for activating HIV expression in latently infected cells in a subject in need thereof.

In another aspect, there is provided use of a compound of Formula (I) or a salt, solvate, or prodrug thereof in combination with one or more anti-HIV viral therapy compounds for treating HIV infection in a subject in need thereof.

In another aspect, there is provided use of a composition comprising a compound of Formula (I) or a salt, solvate, or prodrug thereof in combination with one or more anti-HIV viral therapy compounds for treating HIV infection in a subject in need thereof.

In yet another aspect, there is provided a compound according to Formula (I) or a salt, solvate, or prodrug thereof for use in activating HIV expression in latently infected cells in a subject in need thereof.

In yet another aspect, there is provided a composition comprising a compound according to Formula (I) or a salt, solvate, or prodrug thereof for use in activating HIV expression in latently infected cells in a subject in need thereof.

In yet another aspect, there is provided a compound according to Formula (I) or a salt, solvate, or prodrug thereof in combination with one or more anti-HIV viral therapy compounds for use in treating HIV infection in a subject in need thereof.

In yet another aspect, there is provided a composition comprising a compound according to Formula (I) or a salt, solvate, or prodrug thereof in combination with one or more anti-HIV viral therapy compounds for use in treating HIV infection in a subject in need thereof.

In yet another aspect, there is provided a compound according to Formula (I) or a salt, solvate, or prodrug thereof when used for activating HIV expression in latently infected cells in a subject in need thereof.

In yet another aspect, there is provided a composition comprising a compound according to Formula (I) or a salt, solvate, or prodrug thereof when used for activating HIV expression in latently infected cells in a subject in need thereof.

In yet another aspect, there is provided a compound according to Formula (I) or a salt, solvate, or prodrug thereof in combination with one or more anti-HIV viral therapy compounds when used for treating HIV infection in a subject in need thereof.

In yet another aspect, there is provided a composition comprising a compound according to Formula (I) or a salt, solvate, or prodrug thereof in combination with one or more anti-HIV viral therapy compounds when used for treating HIV infection in a subject in need thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As described herein, activating HIV expression in latently infected cells includes both complete and partial activation of the virus. In one embodiment, activating HIV expression is complete activation. In another embodiment, activating HIV expression is partial activation.

Compounds of the present invention can in certain circumstances be more effective in activating HIV expression in latently infected cells when administered in combination with a bromodomain inhibitor.

The process of gene expression within human cells requires achievement of numerous steps, including the opening of access of DNA heterochromatin compacted with histone proteins into and open structured enchromatin bound by acetylated histone proteins that greatly facilitate access of RNA transcription factors. The HDACi drugs promote access of RNA transcription factors through increasing histone acetylation. However, numerous additional steps are needed to complete the successful expression of proteins, and for latent HIV proviral DNA the viral Tat protein serves as a self-amplifying latency reversing agent by also activating many subsequent steps in gene expression. These include: (1) promotion of nuclear availability of DNA-binding transcription factors such as nuclear factor kappa 1B (NF-kB) that greatly increase the assembly of the RNA transcription complex in T-cells; (2) displacement of negative transcription elongation factor (NTEF) that has two components—the DSIF complex composed of Spt4 and Spt5, which binds to the unphosphorylated form of the carboxyl terminus domain (CTD) of the RNA polymerase II (RNA pol II) inhibiting elongation and the negative elongation factor (NELF) that has four subunits; (3) recruitment of the positive transcription elongation factor b (p-TEFb) that recruits the cyclin dependant kinase 9 (CDK9) and phosphorylates the serine residues on the carboxyl tail of RNA polymerase II (RNA pol II) to enhance the kinetics of processive RNA transcription; (4) recruitment of methyl-transferases that add a m7G-cap structure to the nascent RNA that later serves to assemble ribosomal translation factors for protein synthesis; (5) recruitment of the mRNA splicing factors such as SRSF1 and SRSF2 proteins that promote correct mRNA slicing needed to produce the viral essential regulatory proteins Tat, and Rev; (6) recruitment of other epigenetic proteins that include acetyl- and methyl-transferase complexes such as SET-1b and EZH2 complexes that modify proteins associating with HIV DNA and RNA, and also modify some viral proteins including Tat itself to further modulate viral gene expression.

One of the key regulators of the downstream steps of HIV gene expression are the family of bromodomain and extra-terminal (BET) proteins, including BRD2 and BRD4, that contain two amino-terminal bromodomains with high sequence conservation and an extra terminal (ET) domain. BRD4 carries out various functions in the cell, noted for its stoichiometric association with the active form of P-TEFb, which is mutually exclusive from the binding of P-TEFb in the inhibitory 7SK snRNP complex. BRD4 has been implicated as the factor that recruits P-TEFb for most RNA Pol II-dependent transcriptional elongation by enabling the phosphorylation of serine 2 in the CTD of RNA Pol II. Both bromodomains of BRD4 can simultaneously bind acetylated histones and P-TEFb, particularly in the presence of an HDACi, where histone acetylation increases the recruitment of BRD4:P-TEFb to RNA Pol II. The thienotriazolodiazepine compound JQ1 was developed as a small molecule inhibitor that binds to the acetyl-lysine recognition motifs of bromodomains in BET proteins and inhibits the interaction between BRD4 and P-TEFb.

Because the expression of HIV is regulated at many steps it is likely that combinations of compounds may be required to fully optimise the activation of HIV gene expression achieved by Tat or even by T-cell activation. The present inventors therefore administered novel LRAs in combination with a bromodomain inhibitors, notably JQ1 (+) and PFI1. These combinations were shown to be synergistic.

Bromodomain inhibitors can include any suitable inhibitor such as PFI-1 and JQ1.

The salts of the compounds of Formula (I) are preferably pharmaceutically acceptable, but it will be appreciated that non-pharmaceutically acceptable salts also fall within the scope of the present disclosure, since these are useful as intermediates in the preparation of pharmaceutically acceptable salts.

The term “pharmaceutically acceptable” may be used to describe any pharmaceutically acceptable salt, hydrate or prodrug, or any other compound which upon administration to a subject, is capable of providing (directly or indirectly) a compound of Formula (I) or an active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of Formula (I), such as anhydrous forms, hydrous forms, solvate forms and mixed solvate forms.

Formula (I) is intended to cover, where applicable, solvated as well as unsolvated forms of the compounds. Thus, Formula (I) includes compounds having the indicated structure, including the hydrated or solvated form, as well as the non-hydrated and non-solvated forms.

As used herein, the term “solvate” refers to a complex of variable stoichiometry formed by a solute (in this invention, a compound of Formula (I) or a salt or prodrug thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

A “prodrug” is a compound that may not fully satisfy the structural requirements of the compounds provided herein, but is modified in vivo, following administration to a subject or patient, to produce a compound of Formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

The compounds of Formula (I) and prodrugs thereof may be covalent irreversible or covalent reversible inhibitors of the active site of a protein.

Pharmaceutical compositions may be formulated from compounds according to Formula (I) for any appropriate route of administration including, for example, topical (for example, transdermal or ocular), oral, buccal, nasal, vaginal, rectal or parenteral administration. The term parenteral as used herein includes subcutaneous, intradermal, intravascular (for example, intravenous), intramuscular, spinal, intracranial, intrathecal, intraocular, periocular, intraorbital, intrasynovial and intraperitoneal injection, as well as any similar injection or infusion technique. In certain embodiments, compositions in a form suitable for oral use or parenteral use are preferred. Suitable oral forms include, for example, tablets, troches, lozenges, aqueous or oily suspensions, dispersible powders or granules, emulsions, hard or soft capsules, or syrups or elixirs. For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. The formulations may be present in unit or multi-dose containers such as sealed ampoules or vials. Examples of components are described in Martindale—The Extra Pharmacopoeia (Pharmaceutical Press, London 1993) and Martin (ed.), Remington's Pharmaceutical Sciences.

In the context of this specification the term “administering” and variations of that term including “administer” and “administration”, includes contacting, applying, delivering or providing a compound or composition of the invention to an organism, or a surface by any appropriate means.

For the activation of HIV expression in latently infected cells in a subject in need thereof, the dose of the biologically active compound according to the invention may vary within wide limits and may be adjusted to individual requirements. Active compounds according to the present invention are generally administered in an effective amount. Preferred doses range 5 from about 0.1 mg to about 140 mg per kilogram of body weight per day (e.g. about 0.5 mg to about 7 g per patient per day). The daily dose may be administered as a single dose or in a plurality of doses. The amount of active ingredient that may be combined with the carrier materials to produce a single dosage form will vary depending upon the subject treated and the particular mode of administration. Dosage unit forms will generally contain between about 1 mg to about 500 mg of an active ingredient.

It will be understood, however, that the specific dose level for any particular subject and will depend upon a variety of factors including the activity of the specific compound employed, the age, body weight, general health, sex, diet, time of administration, route of administration, and rate of excretion, drug combination (i.e. other drugs being used to treat the subject), and the severity of the particular disorder undergoing therapy. The dosage will generally be lower if the compounds are administered locally rather than systemically, and for prevention rather than for treatment. Such treatments may be administered as often as necessary and for the period of time judged necessary by the treating physician. A person skilled in the art will appreciate that the dosage regime or therapeutically effective amount of the compound of Formula (I) to be administered may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

It will also be appreciated that different dosages may be required to activate expression of HIV in latently infected cells. An effective amount of an agent is that amount which causes a statistically significant increase in expression of HIV in latently infected cells.

For in vitro analysis, activation of HIV expression in latently infected cells may be determined by using a cell-based screening assay as described in the biological tests defined herein.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of HIV infection and/or associated diseases or their symptoms.

“Anti-HIV viral therapy compound” is used herein to refer to any anti-HIV viral therapy, including but not limited to lamivudine, zidovudine, lopinavir, ritonavir, abacavir, tenofovir, efavirenz, emtricitabine, rilpivirine, dolutegravir, atazanavir, darunavir and raltegravir.

“Subject” includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs.

The compounds of the present invention may be administered along with a pharmaceutical carrier, diluent or excipient as described above.

The methods and compounds described herein are described by the following illustrative and non-limiting examples.

### EXAMPLES

### Synthetic Example 1

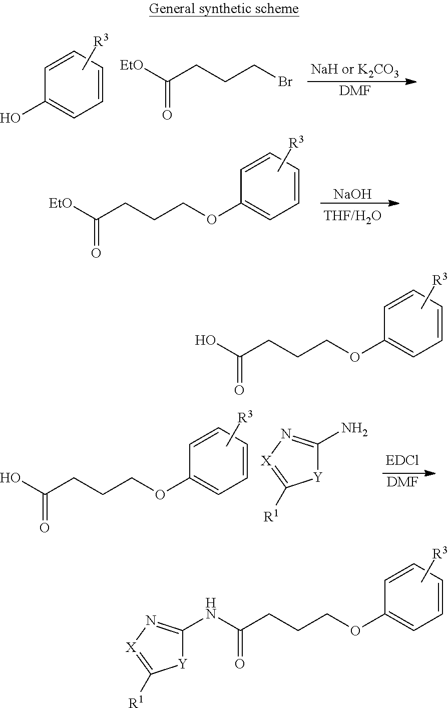

General Description of Chemistry

Compounds of the invention may be prepared following the procedures illustrated below for the preparation of compound E.

Step 1. A phenol (A) is alkylated with an alkyl halide derivative (B) under basic conditions. Typical bases include sodium hydride, sodium hydroxide, caesium carbonate, potassium carbonate. The reaction is generally performed in a solvent such as THF, DMF or acetonitrile and the reaction typically carried out with cooling or may be performed with heating. Catalytic quantities of alkyl iodides (e.g. NaI) may also be added.

Alkylation of the phenol A may also be achieved under Mitsunobu conditions by reacting the phenol with an alcohol derivative (B2) in the presence of a phosphine such as triphenylphosphine and an azodicarboxylate derivative such as diethylazodicarboxylate.

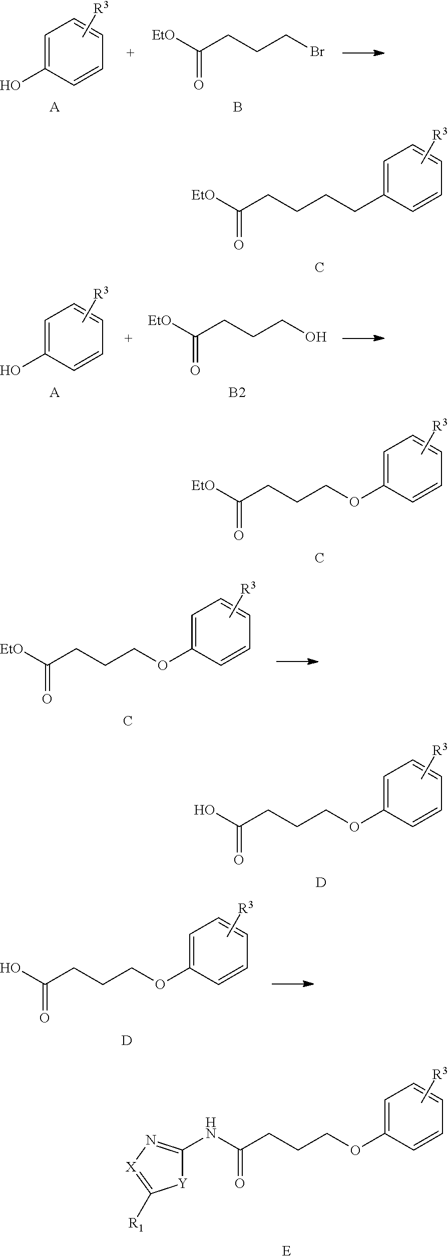

Hydrolysis of the ester C may be achieved under acidic or basic conditions well known to those skilled in the art. The resulting acid D may then be coupled to a heterocyclic amine derivative to afford E under amide coupling conditions. Typical conditions utilise a peptide coupling reagent such as a carbodiimide (e.g. EDCI), a phosphonium derivative (e.g. PyBOP), a uronium species (e.g. TBTU) or related species (e.g. HATU); are conducted with cooling at ambient temperature or with heating; and are performed in solvents such as DMF or dichloroethane.

It will be appreciated that the methods described above are illustrative and the reaction sequences may be conducted in an alternative order to that described above. Further elaboration of the compounds prepared as described above may also be undertaken, using procedures well known to those skilled in the art, to prepare compounds of the present invention.

Representative Synthetic Procedure

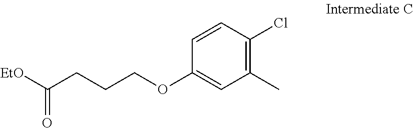 [SMILES: CCOC(=O)CCCOc1ccc(Cl)c(C)c1]

Sodium hydride (60% in mineral oil) (769 mg, 19.4 mmol) was added to stirred solution of 4-chloro-3-methylphenol (2.5 g, 17.4 mmol) in DMF (10 mL) at 00° C. After 15 min of stirring, a solution of ethyl bromobutyrate (3.75 g, 19.4 mmol) in DMF (2 mL) was added dropwise over 1 min at 0° C. The solution was then stirred for 16 h at 20° C. 2N HCl was added and the solution extracted with Et₂O (2×). The organic layer was washed with brine, dried with MgSO₄, and concentrated in vacuo. The crude material was purified by column chromatography eluting with 100% CyHex to 35% EtOAc/CyHex to yield the title compound as an oil (2.9 g, 65%). ¹H NMR (CDCl₃): δ 7.22 (d, J 8.7 Hz, 1H), 6.79-6.77 (m, 1H), 6.69-6.65 (m, 1H), 4.23-4.12 (m, 2H), 4.04-3.96 (m, 2H), 2.57 (m, 2H), 2.35 (s, 3H), 2.16-2.09 (m, 2H), 1.33-1.25 (m, 3H).

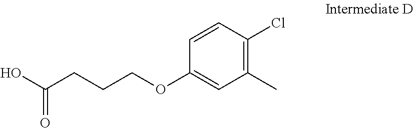

Intermediate C (2.8 g, 10.9 mmol) and LiOH.H₂O (916 mg, 21.9 mmol) in a solution of THF (20 mL) and water (20 mL) was stirred for 4 h at 20° C. The solution was then acidified with 2N HCl and extracted with Et₂O (2×40 mL). The organic layer was washed with brine, dried with MgSO₄, and concentrated in vacuo to give the product as a white solid (2.4 g, 96%). ¹H NMR (d₆-DMSO): δ 7.29-7.21 (m, 2H), 6.78 (s, 1H), 6.70-6.65 (m, 1H), 4.02-3.97 (m, 2H), 2.62-2.56 (m, 2H), 2.17-2.08 (m, 2H).

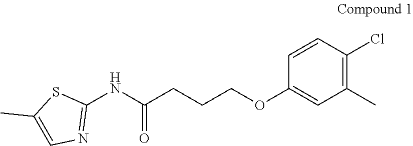

Intermediate D (50 mg, 0.22 mmol), 5-methyl-2-amino thiazole (25 mg, 0.22 mmol), EDCI (42 mg, 0.22 mmol), and DMAP (2.6 mg, 0.02 mmol) in DCE (5 mL) were stirred at 45° C. for 16 h. The reaction was quenched with 10% citric acid solution (10 mL) and extracted with DCM (2×10 mL). The organic layer was then washed with 10% NaHCO₃ solution (1×15 mL), dried (MgSO₄) and concentrated in vacuo. The solid was triturated with Et₂O and filtered off to give a white solid (45 mg, 63%). ¹H NMR (CDCl₃): δ 7.28-7.22 (m, 1H), 7.20-7.19 (m, 1H), 7.12 (1H, s), 6.75 (m, 1H), 6.66 (dd, J 10.9 and 2.2 Hz), 4.06-4.03 (m, 2H), 2.76 (t, J 7.20 Hz, 2H), 2.41 (s, 3H), 2.33 (s, 3H), 2.28-2.23 (m, 2H). MS, m/z=325 [M+H]⁺, 327.

The compounds exemplified below were generated following similar methods to those outlined above.

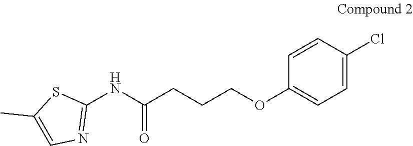 [SMILES: Cc1cnc(NC(=O)CCCOc2ccc(Cl)cc2)s1]

¹H NMR (CDCl₃): δ 7.22 (dd, J 6.7 and 2.2 Hz, 2H), 7.11 (s, 1H), (dd, J 6.8 and 2.2 Hz, 2H), 4.06 (t, J 5.8 Hz, 2H), 2.76 (t, J 7.32 Hz, 2H), 2.42 (s, 3H), 2.31-2.22 (m, 2H). MS, m/z=311 [M+H]⁺, 313.

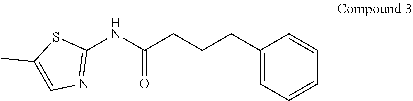 [SMILES: Cc1cnc(NC(=O)CCCc2ccccc2)s1]

¹H NMR (CDCl₃): δ 7.31-7.21 (m, 5H), 6.78 (s, 1H), 2.79-2.71 (m, 2H), 2.55-2.47 (m, 2H), 2.40 (s, 3H), 2.17-2.10 (s, 2H). MS, m/z=261 [M+H]⁺.

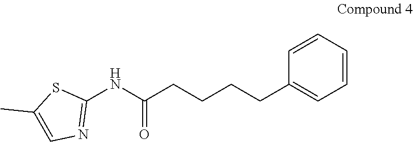 [SMILES: Cc1cnc(NC(=O)CCCCc2ccccc2)s1]

¹H NMR (CDCl₃): δ 7.32-7.17 (m, 5H), 7.03 (s, 1H), 2.68 (t, J 7.4 Hz, 2H), 2.54 (t, J 6.9 Hz, 2H), 2.42 (s, 3H), 1.85-1.70 (m, 4H). MS, m/z=275 [M+H]⁺.

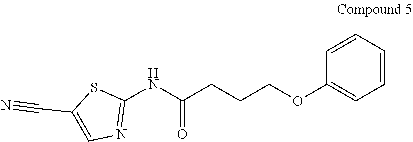 [SMILES: N#Cc1cnc(NC(=O)CCCOc2ccccc2)s1]

¹H NMR (CDCl₃): δ 8.01 (s, 1H), 7.34-7.28 (m, 2H), 7.01-6.90 (m, 3H), 4.12 (t, J 5.5 Hz, 2H), 2.81 (t, J 7.1 Hz, 2H), 2.34-2.25 (m, 2H). MS, m/z=288 [M+H]⁺.

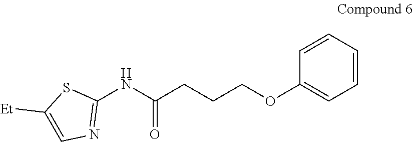 [SMILES: CCc1cnc(NC(=O)CCCOc2ccccc2)s1]

¹H NMR (CDCl₃): δ 7.31-7.25 (m, 2H), 7.15 (s, 1H), 6.98-6.88 (3H, m), 4.09 (t, J 5.8 Hz, 2H), 2.84-2.76 (m, 4H), 2.31-2.23 (m, 2H), 1.32 (t, J 7.5 Hz, 3H). MS, m/z=291 [M+H]⁺.

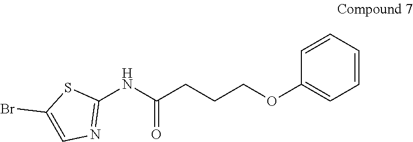 [SMILES: O=C(CCCOc1ccccc1)Nc1ncc(Br)s1]

¹H NMR (CDCl₃): δ 7.42 (s, 1H), 7.42-7.27 (m, 2H), 6.99-6.90 (m, 3H), 4.09 (t, J 5.6 Hz, 2H), 2.77 (t, J 7.1 Hz, 2H), 2.32-2.23 (m, 2H). MS, m/z=341 [M+H]⁺, 343.

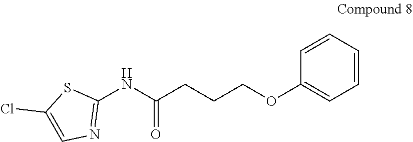 [SMILES: O=C(CCCOc1ccccc1)Nc1ncc(Cl)s1]

¹H NMR (CDCl₃): δ 7.35-7.27 (3H, m), 6.99-6.89 (3H, m), 4.09 (t, J 5.7 Hz, 2H), 2.77 (t, J 7.2 Hz, 2.32-2.23 (m, 2H). MS, m/z=297 [M+H]⁺, 299.

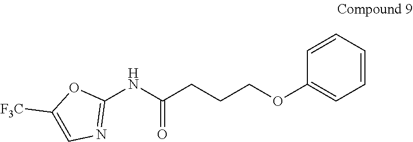 [SMILES: O=C(CCCOc1ccccc1)Nc1ncc(C(F)(F)F)o1]

¹H NMR (CDCl₃): δ 7.48 (s, 1H), 7.33-7.26 (m, 2H), 6.99-6.89 (m, 3H), 4.10-4.02 (m, 2H), 2.74-2.68 (m, 2H), 2.27-2.12 (m, 2H). MS, m/z=315 [M+H]f.

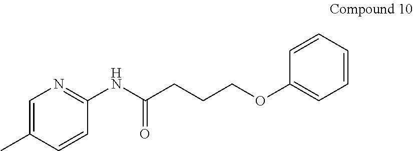 [SMILES: Cc1ccc(NC(=O)CCCOc2ccccc2)nc1]

¹H NMR (CDCl₃): δ 8.53 (bs, 1H), 8.13-8.11 (m, 2H), 7.32-7.26 (m, 2H), 6.98-6.88 (m, 4H), 4.10-4.05 (m, 2H), 2.67-2.62 (2H, m), 2.39 (s, 3H), 2.26-2.21 (m, 2H). MS, m/z=271 [M+H]⁺.

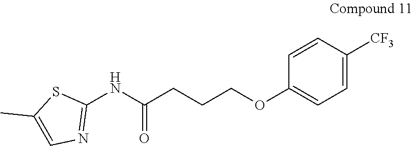 [SMILES: Cc1cnc(NC(=O)CCCOc2ccc(C(F)(F)F)cc2)s1]

¹H NMR (CDCl₃): δ 7.55 (d, J 8.9 Hz, 2H), 7.11 (s, 1H), 6.95 (d, J 8.6 Hz, 2H), 4.14 (t, J 5.9 Hz, 2H), 2.77 (t, J 7.2 Hz, 2H), 2.41 (s, 3H), 2.34-2.27 (m, 2H). MS, m/z=345 [M+H]⁺.

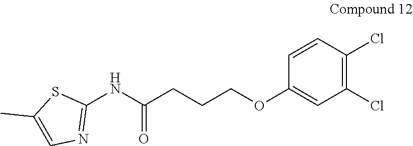 [SMILES: Cc1cnc(NC(=O)CCCOc2ccc(Cl)c(Cl)c2)s1]

¹H NMR (CDCl₃): δ 7.33-7.29 (m, 2H), 7.10 (d, J 0.9 Hz, 1H), 6.98 (d, J 2.7 Hz, 1H), 6.73 (dd, J 8.7 and 3.0 Hz, 1H), 4.06 (t, J 7.1 Hz, 2H), 2.77 (t, J 7.2 Hz, 2H), 2.43 (3H, s), 2.30-2.25 (m, 2H). MS, m/z=345 [M+H]⁺, 347.

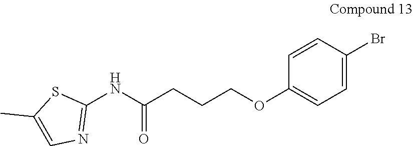 [SMILES: Cc1cnc(NC(=O)CCCOc2ccc(Br)cc2)s1]

¹H NMR (CDCl₃): δ 7.37 (d, J 9.1 Hz, 2H), 7.11 (s, 1H), 6.76 (d, J 9.0 Hz, 2H), 4.06 (t, J 5.9 Hz, 2H), 2.75 (t, J 7.2 Hz, 2H), 2.41 (s, 3H), 2.31-2.22 (m, 2H). MS, m/z=355 [M+H]⁺, 357.

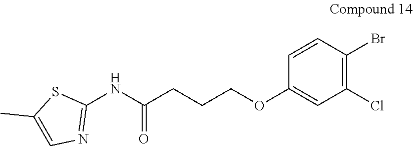 [SMILES: Cc1cnc(NC(=O)CCCOc2ccc(Br)c(Cl)c2)s1]

¹H NMR (CDCl₃): δ 7.49-7.44 (m, 2H), 7.09 (s, 1H), 7.01-6.99 (s, m), 6.70-6.64 (m, 2H), 4.06 (t, J 5.8 Hz, 2H), 2.76-2.71 (m, 2H), 2.42 (s, 3H), 2.31-2.25 (m, 2H). MS, m/z=389 [M+H]⁺, 391.

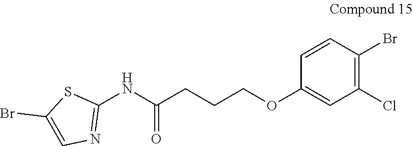 [SMILES: O=C(CCCOc1ccc(Br)c(Cl)c1)Nc1ncc(Br)s1]

¹H NMR (d₆-DMSO): δ 7.62 (d, J 8.9 Hz, 1H), 7.53 (s, 1H), 7.19 (d, J 2.9 Hz, 1H), 6.86 (dd, J 8.9 and 2.8 Hz, 1H), 4.04 (t, J 6.2 Hz, 2H), 2.60 (t, J 6.9 Hz, 2H), 2.08-1.98 (m, 2H). MS, m/z=455 [M+H]⁺, 453, 457.

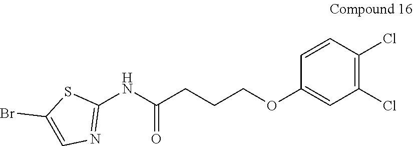 [SMILES: O=C(CCCOc1ccc(Cl)c(Cl)c1)Nc1ncc(Br)s1]

¹H NMR (d₆-DMSO): δ 7.55-7.48 (m, 2H), 7.18 (d, J 2.6 Hz, 1H), 6.95-6.91 (m, 1H), 4.04 (t, J 6.3 Hz, 2H), 2.60 (t, J 7.1 Hz, 2H), 2.08-2.01 (m, 2H). MS, m/z=411 [M+H]⁺, 409, 413.

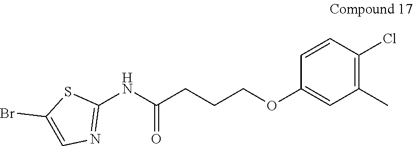 [SMILES: Cc1cc(OCCCC(=O)Nc2ncc(Br)s2)ccc1Cl]

¹H NMR (d₆-DMSO): δ 7.53 (s, 1H), 7.27 (d, J 8.7 Hz, 1H), 6.89 (d, J 3.0 Hz), 6.76 (dd, J 8.5 and 2.8 Hz, 1H), 3.98 (t, J 6.2 Hz, 2H), 2.59 (t, J 7.2 Hz, 2H), 2.07-1.98 (m, 2H). MS, m/z=391 [M+H]⁺, 389.

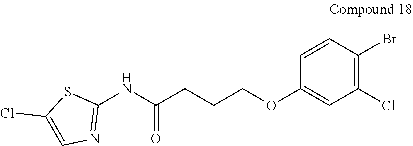 [SMILES: O=C(CCCOc1ccc(Br)c(Cl)c1)Nc1ncc(Cl)s1]

¹H NMR (d₆-DMSO): δ 7.62 (d, J 8.9 Hz, 1H), 7.49 (s, 1H), 7.18 (d, J 2.9 Hz, 1H), 6.86 (dd, J 8.9 and 2.88 Hz, 1H), 4.04 (t, J 6.2 Hz, 2H), 2.60 (t, J 7.3 Hz, 2H), 2.08-2.01 (m, 2H). MS, m/z=411 [M+H]⁺, 409, 413.

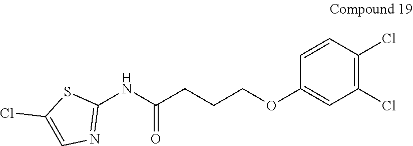 [SMILES: O=C(CCCOc1ccc(Cl)c(Cl)c1)Nc1ncc(Cl)s1]

¹H NMR (d₆-DMSO): δ 7.50-7.47 (m, 2H), 7.16 (d, J 2.9 Hz, 1H), 6.90 (dd, J 8.9 and 2.9 Hz, 1H), 4.01 (t, J 6.2 Hz, 2H), 2.58 (t, J 7.2 Hz, 2H), 2.03-1.98 (m, 2H). MS, m/z=365 [M+H]⁺, 367.

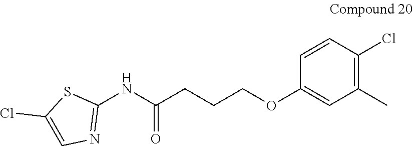 [SMILES: Cc1cc(OCCCC(=O)Nc2ncc(Cl)s2)ccc1Cl]

¹H NMR (d₆-DMSO): δ 7.47 (s, 1H), 7.25 (d, J 8.8 Hz, 1H), 6.87 (d, J 2.9 Hz, 1H), 7.73 (dd, J 8.9 and 3.1 Hz), 3.96 (t, J 6.2 Hz, 2H), 2.58 (t, J 7.3 Hz, 2H), 2.24 (3H, s), 2.05-1.96 (2H, m). MS, m/z=345 [M+H]⁺, 347.

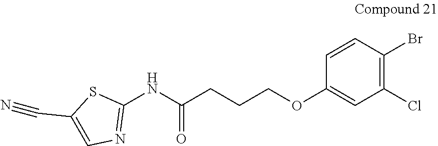 [SMILES: N#Cc1cnc(NC(=O)CCCOc2ccc(Br)c(Cl)c2)s1]

¹H NMR (d₆-DMSO): δ 8.34 (s, 1H), 7.60 (d, J 8.9 Hz, 1H), 7.15 (d, J 2.1 Hz, 1H), 6.83 (dd, J 9.1 and 2.7 Hz, 1H), 4.03 (t, J 5.9 Hz, 2H), 2.65 (t, J 6.2 Hz, 2H), 2.08-1.99 (m, 2H). MS, m/z=400 [M+H]⁺, 402.

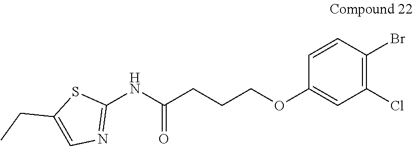 [SMILES: CCc1cnc(NC(=O)CCCOc2ccc(Br)c(Cl)c2)s1]

¹H NMR (d₆-DMSO): δ 7.59 (d, J 8.9 Hz, 1H), 7.16 (d, J 2.9 Hz, 1H), 7.11 (s, 1H), 6.83 (dd, J 8.9 and 2.9 Hz, 1H), 4.01 (t, J 6.3 Hz, 2H), 2.72 (q, J 7.5 Hz, 2H), 2.54 (t, J 7.4 Hz, 2H), 2.04-1.95 (m, 2H), 1.19 (t, J 7.5 Hz, 3H). MS, m/z=403 [M+H]⁺, 405.

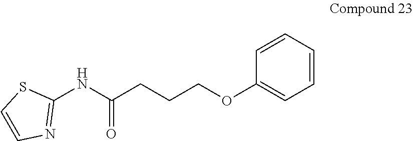 [SMILES: O=C(CCCOc1ccccc1)Nc1nccs1]

¹H NMR (CDCl₃): δ 7.50 (s, 1H), 7.29-7.23 (m, 2H), 6.98-6.85 (4H, m), 4.09 (t, J 5.9 Hz, 2H), 2.82-2.77 (m, 2H), 2.31-2.25 (m, 2H). MS, m/z=263 [M+H]⁺.

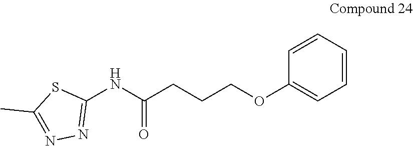 [SMILES: Cc1nnc(NC(=O)CCCOc2ccccc2)s1]

¹H NMR (d₆-DMSO): δ 7.30-7.24 (m, 1H), 6.94-6.88 (m, 1H), 4.00 (t, J 6.3 Hz, 2H), 2.66-2.60 (m, 5H), 2.09-2.00 (m, 2H). MS, m/z=278 [M+H]⁺.

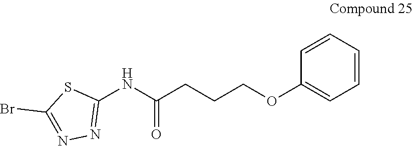 [SMILES: O=C(CCCOc1ccccc1)Nc1nnc(Br)s1]

¹H NMR (d₆-DMSO): δ 7.27-7.22 (m, 2H), 6.91-6.85 (m, 3H), 3.98 (t, J 6.3 Hz, 2H), 2.68 (t, J 7.3 Hz, 2H), 2.07-2.00 (m, 2H). MS, m/z=342 [M+H]⁺, 344.

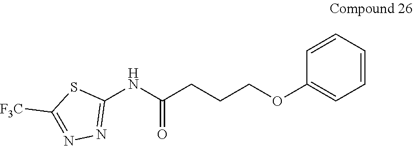 [SMILES: O=C(CCCOc1ccccc1)Nc1nnc(C(F)(F)F)s1]

¹H NMR (d₆-DMSO): δ 7.30-7.24 (m, 2H), 6.94-6.87 (m, 3H), 4.04 (t, J 6.3 Hz, 2H), 2.74 (t, J 7.3 Hz, 2H), 2.13-2.04 (m, 2H). MS, m/z=332 [M+H]⁺.

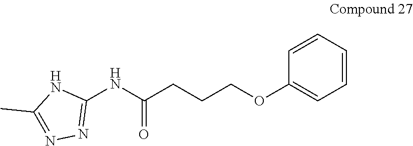 [SMILES: Cc1nnc(NC(=O)CCCOc2ccccc2)[nH]1]

¹H NMR (d₆-DMSO): δ 7.40 (brs, 1H), 7.28-7.22 (m, 2H), 6.92-6.87 (m, 3H), 4.03-3.98 (m, 2H), 3.07-3.02 (m, 2H), 2.07-2.03 (m, 5H). MS, m/z=261 [M+H]⁺.

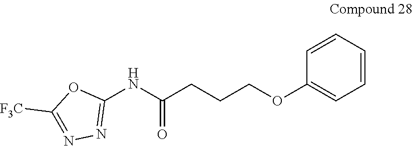 [SMILES: O=C(CCCOc1ccccc1)Nc1nnc(C(F)(F)F)o1]

¹H NMR (d₆-DMSO): δ 7.28-7.23 (m, 2H), 6.92-6.88 (m, 3H), 3.99 (t, J 6.3 Hz, 2H), 2.63 (t, J 7.1 Hz, 2H), 2.06-1.96 (m, 2H). MS, m/z=316 [M+H]⁺.

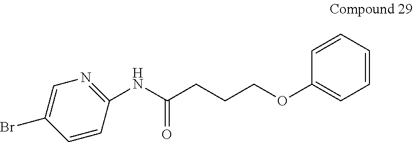 [SMILES: O=C(CCCOc1ccccc1)Nc1ccc(Br)cn1]

¹H NMR (d₆-DMSO): δ 10.65 (s, 1H), 8.39 (d, J 2.5 Hz, 1H), 8.07 (d, J 8.9 Hz, 1H), 7.97 (dd, J 8.9 and 2.6 Hz, 1H), 7.28-7.22 (m, 2H), 6.91-6.87 (m, 3H), 3.97 (t, J 6.4 Hz, 2H), 2.56 (t, J 7.4 Hz, 2H), 2.04-1.95 (m, 2H). MS, m/z=335 [M+H]⁺, 337.

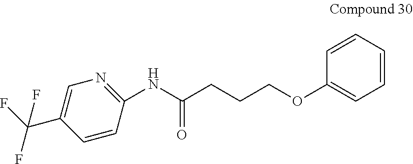 [SMILES: O=C(CCCOc1ccccc1)Nc1ccc(C(F)(F)F)cn1]

¹H NMR (d₆-DMSO): δ 8.69 (s, 1H), 8.30 (d, J 8.9 Hz, 1H), 8.16 (dd, J 8.9 and 2.7 Hz), 7.29-7.24 (m, 2H), 6.94-6.89 (m, 3H), 4.01 (t, J 6.33 Hz, 2H), 2.63 (t, J 7.3 Hz, 2H), 2.07-2.00 (m, 2H). MS, m/z=325 [M+H]⁺.

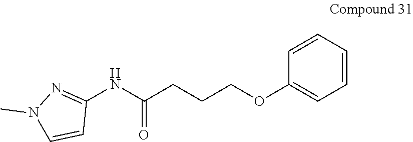 [SMILES: Cn1ccc(NC(=O)CCCOc2ccccc2)n1]

¹H NMR (d₆-DMSO): δ 10.35 (s, 1H), 7.51 (d, J 2.2 Hz, 1H), 7.30-7.24 (m, 2H), 6.93-6.89 (m, 3H), 6.43 (s, 1H), 3.97 (t, J 6.4 Hz, 2H), 3.72 (s, 3H), 2.45 (t, J 7.4 Hz, 2H), 2.03-1.94 (m, 2H). MS, m/z=260 [M+H]⁺.

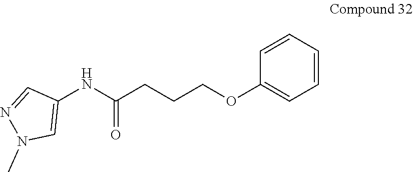 [SMILES: Cn1cc(NC(=O)CCCOc2ccccc2)cn1]

¹H NMR (d₆-DMSO): δ 9.90 (s, 1H), 7.82 (s, 1H), 7.34 (s, 1H), 7.28-7.23 (m, 2H), 6.92-6.87 (m, 2H), 3.96 (t, J 6.4 Hz, 2H), 3.75 (s, 3H), 2.39 (t, J 7.26 Hz, 2H), 2.02-1.93 (m, 2H).

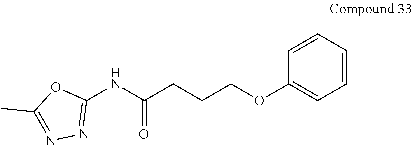 [SMILES: Cc1nnc(NC(=O)CCCOc2ccccc2)o1]

¹H NMR (d₆-DMSO): δ 7.62 (s, 1H), 7.29-7.22 (m, 2H), 6.93-6.87 (m, 3H), 4.04-3.95 (m, 2H), 2.85 (t, J 7.3 Hz, 2H), 2.21 (s, 3H), 2.07-1.98 (m, 2H). MS, m/z=262 [M+H]⁺.

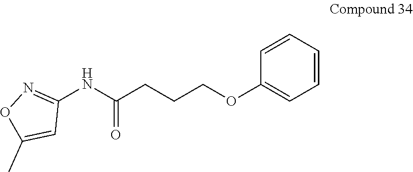 [SMILES: Cc1cc(NC(=O)CCCOc2ccccc2)no1]

¹H NMR (d₆-DMSO): δ 10.86 (s, 1H), 7.28-7.22 (m, 2H), 6.92-6.87 (m, 3H), 6.61 (s, 1H), 3.96 (t, J 6.4 Hz, 2H), 2.52-2.48 (m, 2H), 2.34 (s, 3H), 2.02-1.93 (m, 2H). MS, m/z=261 [M+H]⁺.

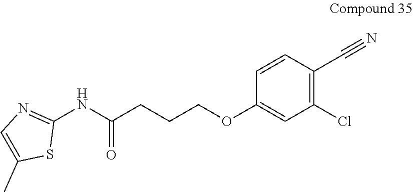 [SMILES: Cc1cnc(NC(=O)CCCOc2ccc(C#N)c(Cl)c2)s1]

¹H NMR (d₆-DMSO): δ 7.85 (d, J 8.8 Hz, 1H), 7.28 (d, J 2.4 Hz, 1H), 7.08-7.03 (m, 2H), 4.13 (t, J 6.2 Hz, 2H), 2.55 (t, J 7.4 Hz, 2H), 2.31 (s, 3H), 2.07-1.98 (m, 2H). MS, m/z=336 [M+H]⁺.

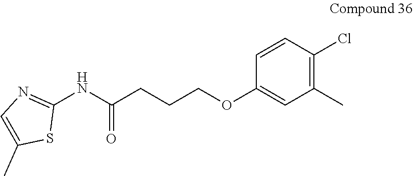 [SMILES: Cc1cnc(NC(=O)CCCOc2ccc(Cl)c(C)c2)s1]

¹H NMR (d₆-DMSO): δ 7.11 (d, J 8.4 Hz, 1H), 6.88 (d, J 2.4 Hz, 1H), 6.71 (dd, J 8.37 and 2.5 Hz), 4.00 (t, J 6.0 Hz, 2H), 2.59 (t, J 7.3 Hz, 2H), 2.31 (s, 3H), 2.17-2.06 (m, 2H). MS, m/z=325 [M+H]⁺.

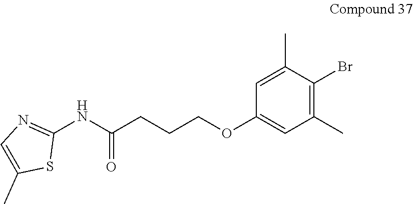 [SMILES: Cc1cnc(NC(=O)CCCOc2cc(C)c(Br)c(C)c2)s1]

¹H NMR (d₆-DMSO): δ 7.10 (s, 1H), 7.76 (2H, s), 3.97 (t, J 6.2 Hz, 2H), 2.58-2.55 (m, 2H), 2.33-2.30 (m, 9H), 2.06-1.96 (m, 2H). MS, m/z=383 [M+H]⁺, 385.

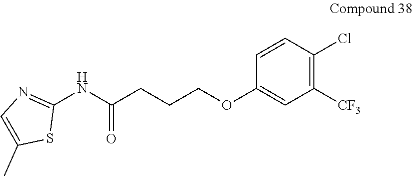 [SMILES: Cc1cnc(NC(=O)CCCOc2ccc(Cl)c(C(F)(F)F)c2)s1]

¹H NMR (d₆-DMSO): δ 7.61 (d, J 8.3 Hz, 1H), 7.27-7.22 (m, 3H), 7.10 (s, 1H), 4.10 (t, J 6.15 Hz, 2H), 2.57 (t, J 7.3 Hz, 2H), 2.33 (s, 3H), 2.09-2.00 (m, 2H). MS, m/z=379 [M+H]⁺.

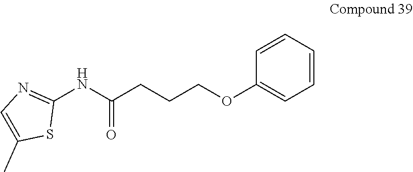 [SMILES: Cc1cnc(NC(=O)CCCOc2ccccc2)s1]

This compound was purchased from Enamine.

### Synthetic Example 2

General Procedure A=Intermediate C

General Procedure B=Intermediate D

General Procedure C=Intermediate E

General Chemistry Procedures.

Flash chromatography was performed with silica gel 60 (particle size 0.040-0.063 μm). NMR spectra were recorded on a Bruker Avance DRX 300 with the solvents indicated (¹H NMR at 300 MHz). Chemical shifts are reported in ppm on the δ scale and referenced to the appropriate solvent peak (Chloroform range 7.26-7.27 ppm). NMR spectra were processed using ACD/NMR Processor Academic Edition, version 12.01, Advanced Chemistry Development, Inc., Toronto, ON, Canada, www.acdlabs.com, 2010. LCMS were recorded on an Agilent G6120B MSD using a 1260 Infinity Diode Array Detector. LCMS conditions used to assess purity of compounds for this system were as follows: Poroshell 120 EC-C18, 3.0×50 mm 2.7 Micron; injection volume: 5 uL; gradient: 5-100% B over 3 min (solvent A, water 0.1% formic acid; solvent B: AcCN 0.1% formic Acid); flowrate: 0.8 ml/min; 254 nm. LCMS were also recorded on a Waters ZQ 3100 using a 2996 diode array detector. LCMS conditions used to assess purity of compounds for this system were as follows: column, XBridge™ C18 5 μm 4.6 mm×100 mm; injection volume 10 μL; gradient, 10-100% B over 10 min (solvent A, water 0.1% formic acid; solvent B, AcCN 0.1% formic acid); flow rate. 1.5 mL/min; detection, 100-600 nm.

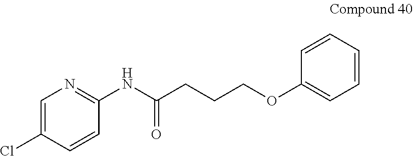 [SMILES: O=C(CCCOc1ccccc1)Nc1ccc(Cl)cn1]

This compound was purchased commercially.

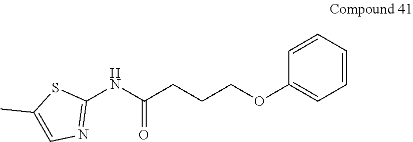 [SMILES: Cc1cnc(NC(=O)CCCOc2ccccc2)s1]

This compound was purchased commercially.

The below examples were generated following similar methods to those outlined to the general protocol outlined in Synthetic Example 1.

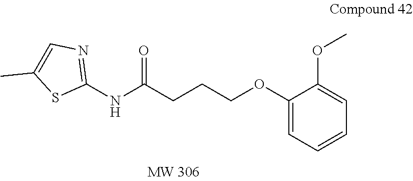

General Procedure C was followed using WIN-330-170-01 (30 mg, 0.14 mmol) and 5-methyl-2-aminothiazole (16 mg, 0.14 mmol) to obtain WIN-330-171-02 as a white solid (26 mg, 59%). ¹H NMR (CDCl₃): δ 7.16 (q, J 1.3 Hz, 1H), 6.80-7.02 (m, 4H), 4.14 (t, J 5.94 Hz, 2H), 3.84 (s, 3H), 2.80 (t, J 7.2 Hz, 2H), 2.4 (d, J 1.3 Hz, 3H), 2.25-2.35 (m, 2H). MS, m/z=307 (100) [M+H]⁺.

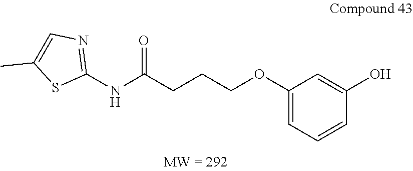 [SMILES: Cc1cnc(NC(=O)CCCOc2cccc(O)c2)s1]

General Procedure C was followed using WIN-330-152-03 (40 mg, 0.20 mmol) and 5-methyl-2-aminothiazole (23 mg, 0.20 mmol). Residue was then purified via preparatory LCMS using a gradient of 95% water/ACN to 100% ACN/water to obtain WIN-330-153-03 as a white solid (2.5 mg, 4%). ¹H NMR (d6-Acetone): δ 7.02-7.11 (m, 2H), 6.38-6.46 (m, 3H), 4.04 (t, J 6.2 Hz, 2H), 2.74 (t, J 7.4 Hz, 2H), 2.37 (d, J 1.1 Hz, 3H), 2.11-2.23 (m, 2H) MS, m/z=293 (100) [M+H]⁺.

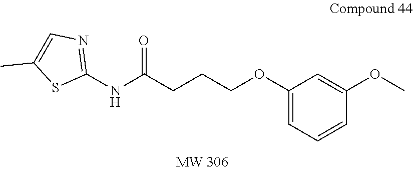

General Procedure C was followed using WIN-330-152-02 (32 mg, 0.15 mmol) and 5-methyl-2-aminothiazole (17 mg, 0.15 mmol) to obtain WIN-330-153-02 as a white solid (40 mg, 86%). ¹H NMR (CDCl₃): δ 7.12-7.21 (m, 2H), 6.41-6.55 (m, 3H), 4.08 (t, J 5.8 Hz, 2H), 3.78 (s, 3H), 2.78 (t, J 7.3 Hz, 2H), 2.40 (d, J 1.1 Hz, 3H), 2.19-2.33 (m, 2H). MS, m/z=307 (100) [M+H]⁺.

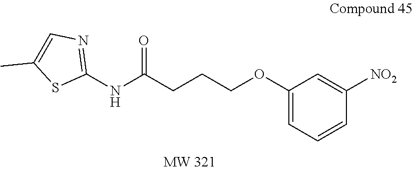 [SMILES: Cc1cnc(NC(=O)CCCOc2cccc([N+](=O)[O-])c2)s1]

General Procedure C was followed using WIN-330-189-02 (459 mg, 2.04 mmol) and 5-methyl-2-aminothiazole (232 mg, 2.04 mmol) to obtain WIN-330-189-02 as a white solid (429 mg, 65%). ¹H NMR (CDCl₃): δ 7.81-7.87 (m, 1H), 7.73 (t, J 2.2 Hz, 1H), 7.37-7.49 (m, 1H), 7.17-7.25 (m, 1H), 7.10 (d, J 1.1 Hz, 1H), 4.18 (t, J 5.8 Hz, 2H), 2.74 (t, J 7.2 Hz, 2H), 2.43 (d, J 1.3 Hz, 3H), 2.24-2.40 (m, 2H). MS, m/z=322 (100) [M+H]J.

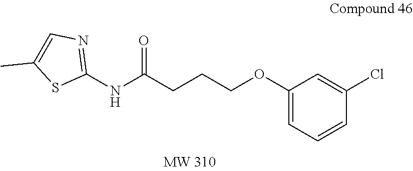 [SMILES: Cc1cnc(NC(=O)CCCOc2cccc(Cl)c2)s1]

General Procedure C was followed using WIN-330-171-02 (30 mg, 0.14 mmol) and 5-methyl-2-aminothiazole (16 mg, 0.14 mmol) to obtain WIN-330-171-03 as a white solid (10 mg, 23%). ¹H NMR (CDCl₃): δ 7.14-7.24 (m, 1H), 7.09-7.14 (m, 1H), 6.94 (ddd, J 8.0, 1.8, 1.0 Hz, 1H), 6.89 (t, J 2.2 Hz, 1H), 6.77 (ddd, J 8.4, 2.4, 0.9 Hz, 1H), 4.08 (t, J 5.8 Hz, 2H), 2.75 (t, J 7.3 Hz, 2H), 2.42 (d, J 1.1 Hz, 3H), 2.22-2.34 (m, 2H) MS, m/z=311 (100) [M+H]⁺, 313 (30).

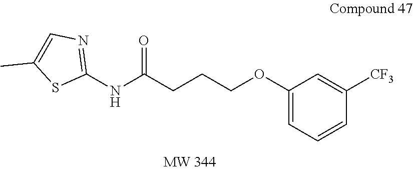 [SMILES: Cc1cnc(NC(=O)CCCOc2cccc(C(F)(F)F)c2)s1]

General Procedure C was followed using WIN-330-171-02 (30 mg, 0.12 mmol) and 5-methyl-2-aminothiazole (14 mg, 0.12 mmol) to obtain WIN-330-158-02 as a white solid (25.2 mg, 68%). ¹H NMR (CDCl₃): δ 7.34-7.44 (m, 1H), 7.17-7.26 (m, 1H), 7.09-7.16 (m, 2H), 7.05 (dd, J 8.1, 2.4 Hz, 1H), 4.14 (t, J 5.7 Hz, 2H), 2.79 (t, J 7.3 Hz, 2H), 2.41 (d, J 1.3 Hz, 3H), 2.21-2.38 (m, 2H). MS, m/z=345 (100) [M+H]⁺.

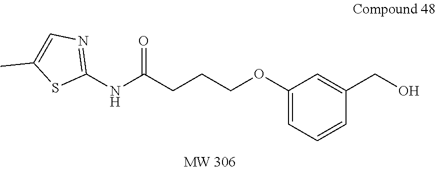 [SMILES: Cc1cnc(NC(=O)CCCOc2cccc(CO)c2)s1]

General Procedure C was followed using WIN-330-164-02 (32 mg, 0.15 mmol) and 5-methyl-2-aminothiazole (17 mg, 0.15 mmol) to obtain WIN-330-164-02 as a white solid (12 mg, 68%) in 60% purity. 6 mg of this crude product was then purified via preparatory HPLC using a gradient of 95% water/ACN to 100% ACN/water to obtain WIN-330-164-02 as a white solid (1.2 mg, 5%). ¹H NMR (CDCl₃): δ 7.25 (d, J 7.92 Hz, 2H), 7.08-7.14 (m, 1H), 6.89-6.98 (m, 2H), 6.76-6.87 (m, 1H), 4.67 (s, 2H), 4.09 (t, J 5.8 Hz, 2H), 2.71 (t, J 7.2 Hz, 2H), 2.42 (d, J 1.3 Hz, 3H), 2.12-2.35 (m, 2H). MS, m/z=307 (100) [M+H]⁺.

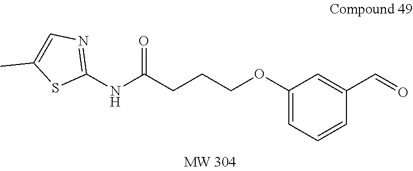 [SMILES: Cc1cnc(NC(=O)CCCOc2cccc(C=O)c2)s1]

WIN-330-164-02 (27 mg, 0.090 mmol) was dissolved in DCM (1 ml) containing 4 Å molecular sieves under N₂ atmosphere. PCC (57 mg, 0.26 mmol) was then added and reaction stirred for 3 h. The reaction was then diluted with additional DCM (10 ml) and filtered through Celite and solvent removed in vacuo. The crude residue was then purified via preparatory HPLC using a gradient of 95% water/ACN to 100% ACN/water to obtain WIN-330-166-01 as a white solid (1.2 g, 4.47%). ¹H NMR (CDCl₃): δ 9.97 (s, 1H), 7.42-7.52 (m, 2H), 7.39 (dd, J 2.0, 1.1 Hz, 1H), 7.08-7.21 (m, 2H), 4.16 (t, J 5.9 Hz, 2H), 2.76 (t, J 7.2 Hz, 2H), 2.42 (d, J 1.3 Hz, 3H), 2.31 (quin, J 6.6 Hz, 2H). MS, m/z=305 (100) [M+H]⁺.

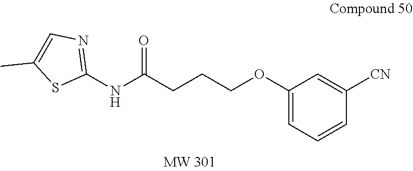 [SMILES: Cc1cnc(NC(=O)CCCOc2cccc(C#N)c2)s1]

General Procedure C was followed using WIN-330-157-01 (30 mg, 0.15 mmol) and 5-methyl-2-aminothiazole (17 mg, 0.15 mmol) to obtain WIN-330-158-01 as a white solid (12 mg, 27%). ¹H NMR (CDCl₃): δ 7.37 (td, J 7.76, 0.99 Hz, 1H), 7.25 (dt, J 7.7, 1.2 Hz, 1H), 7.07-7.16 (m, 3H), 4.12 (t, J 5.9 Hz, 2H), 2.75 (t, J 7.0 Hz, 2H), 2.43 (d, J 1.3 Hz, 3H), 2.24-2.36 (m, 2H). MS, m/z=302 (100) [M+H]⁺.

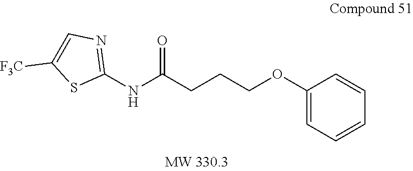 [SMILES: O=C(CCCOc1ccccc1)Nc1ncc(C(F)(F)F)s1]

General Procedure C was followed using 4-phenoxybutanoic acid (26 mg, 0.14 mmol) and 5-(trifluoromethyl)thiazol-2-amine (20 mg, 0.12 mmol) to obtain WIN-321-098-01 as a white solid (12 mg, 31%). ¹H NMR (CDCl₃): δ 7.83 (s, 1H), 7.24-7.33 (m, 2H), 6.93-7.02 (m, 1H), 6.85-6.93 (m, 2H), 4.11 (t, J 5.6 Hz, 2H), 2.81 (t, J 7.0 Hz, 2H), 2.24-2.37 (m, 2H). MS, m/z=331 (100) [M+H]⁺.

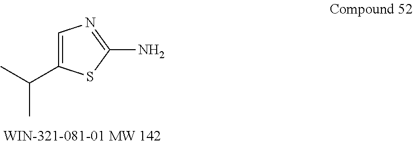

To a stirred mixture of 3-methylbutanal (2.5 g, 29 mmol) in diethyl ether/dioxane (25 ml, 0.10 ml) at −5° C. was added bromine (1.64 mL, 32 mmol) over 2 h. After sustaining the bromine colour (1 h), it was neutralised with sat NaHCO₃(aq) (15 ml). The organic layer was then separated and washed with water (2×20 ml), brine (2×20 ml), dried with Na₂SO₄, filtered and concentrated in vacuo to obtain a crude residue. This residue was then added directly to a stirred solution of thiourea (2.21 g, 29 mmol) in THF (30 ml) and refluxed for 16 h. The reaction was then cooled to 20° C. and quenched with sat NaHCO₃(aq) (15 ml). The THF was evaporated in vacuo and then residue dissolved in 35 ml of ethyl acetate and washed with water (2×20 ml), brine (2×20 ml), dried with Na₂SO₄, filtered and concentrated in vacuo to obtain a crude residue. The crude residue was then purified by column chromatography (100% CyHex to 60% EtOAc/CyHex) to obtain WIN-321-081-01 as an oil (1.47 g, 36%). ¹H NMR (CDCl₃): δ 6.73 (d, J 1.1 Hz, 1H), 3.00 (td, J 6.8, 1.10 Hz, 1H), 1.27 (d, J 6.8 Hz, 6H). MS, m/z=143 (100) [M+H]⁺.

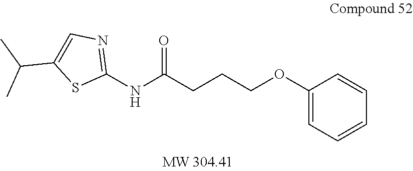 [SMILES: CC(C)c1cnc(NC(=O)CCCOc2ccccc2)s1]

General Procedure C was followed using 4-phenoxybutanoic acid (40 mg, 0.22 mmol) and WIN-321-081-01 (38 mg, 0.27 mmol) to obtain WIN-321-083-01 as a white solid (43 mg, 64%). ¹H NMR (CDCl₃): δ 7.28-7.32 (m, 1H), 7.24-7.28 (m, 1H), 7.14 (d, J 0.9 Hz, 1H), 6.85-7.01 (m, 3H), 4.06-4.13 (m, 2H), 3.15 (td, J 6.8, 0.9 Hz, 1H), 2.81 (t, J 7.3 Hz, 2H), 2.22-2.34 (m, 3H), 1.35 (d, J 7.0 Hz, 6H). MS, m/z=305 (100) [M+H]⁺.

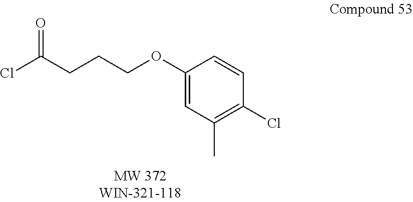

BES-AA0-986-B1 (100 mg, 0.44 mmol) was dissolved in SOCl₂ (3.67 ml, 50 mmol) and refluxed for 4 h. The SOCl₂ was then removed in vacuo to obtain WIN-321-118 (105 mg, 97%) as a brown solid which was used in the next step without further purification.

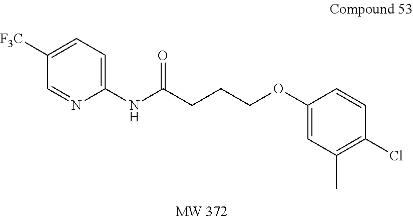 [SMILES: Cc1cc(OCCCC(=O)Nc2ccc(C(F)(F)F)cn2)ccc1Cl]

5-(Trifluoromethyl)pyridin-2-amine (65 mg, 0.40 mmol) was dissolved in pyridine (2 ml) followed by the addition of WIN-321-118 (33 mg, 0.13 mmol) and heated at reflux for 3 d under N₂. The solvent was then removed in vacuo and the residue dissolved in DCM and washed with NaHCO₃ (10 ml), water (10 ml), brine (10 ml), dried with Na₂SO₄, filtered and concentrated in vacuo. The crude residue was then purified by column chromatography (100% CyHex to 40% EtOAc/CyHex) to obtain WIN-321-175-01 as clear crystals (44 mg, 88%). ¹H NMR (CDCl₃): δ 8.55 (s, 1H), 8.28-8.42 (m, 2H), 7.96 (dd, J 8.8, 2.4 Hz, 1H), 7.23 (d, J 8.6 Hz, 1H), 6.79 (d, J 2.9 Hz, 1H), 6.69 (dd, J 8.5, 2.8 Hz, 1H), 4.05 (t, J 5.8 Hz, 2H), 2.68 (t, J 7.0 Hz, 2H), 2.34 (s, 3H), 2.15-2.31 (m, 2H). MS, m/z=373 (100) [M+H]⁺, 375 (30).

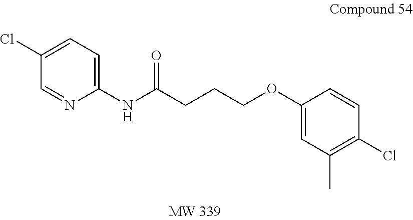 [SMILES: Cc1cc(OCCCC(=O)Nc2ccc(Cl)cn2)ccc1Cl]

General Procedure C was followed using BES-AA0-986-B1 (40 mg, 0.17 mmol) and 5-chloropyridin-2-amine (23 mg, 0.18 mmol) to obtain WIN-321-112-01 as a white solid (13 mg, 22%). ¹H NMR (CDCl₃): δ 8.19-8.25 (m, 2H), 8.10 (br s, 1H), 7.65-7.72 (m, 1H), 7.23 (d, J 8.6 Hz, 1H), 6.79 (d, J 2.9 Hz, 1H), 6.65-6.72 (m, 1H), 4.04 (t, J 5.9 Hz, 2H), 2.63 (t, J 7.2 Hz, 2H), 2.34 (s, 3H), 2.16-2.30 (m, 2H), MS, m/z=339 (100) [M+H]⁺, 341 (30).

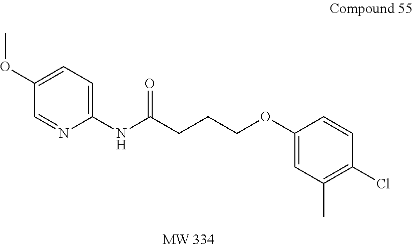 [SMILES: COc1ccc(NC(=O)CCCOc2ccc(Cl)c(C)c2)nc1]

General Procedure C was followed using WIN-321-128-01 (20 mg, 0.090 mmol) and 2-Amino-5-methoxypyridine (11 mg, 0.090 mmol) to obtain WIN-330-197-01 as a white solid (10 mg, 34%). ¹H NMR (CDCl₃): δ 8.17 (d, J 9.0 Hz, 1H), 8.08 (br s, 1H), 7.97 (dd, J 3.1, 0.44 Hz, 1H), 7.30 (d, J 2.9 Hz, 1H), 7.22 (d, J 8.8 Hz, 1H), 6.79 (d, J 3.1 Hz, 1H), 6.68 (dd, J 8.8, 3.1 Hz, 1H), 4.04 (t, J 5.9 Hz, 2H), 3.86 (s, 3H), 2.60 (t, J 7.2 Hz, 2H), 2.34 (s, 3H), 2.06-2.27 (m, 2H). MS, m/z=335 (100) [M+H]⁺.

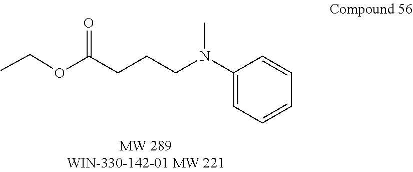 [SMILES: CCOC(=O)CCCN(C)c1ccccc1]

General Procedure A was followed using N-methylaniline (166 μl, 1.54 mmol) and ethyl bromobutyrate (148 μl, 1.03 mmol) to obtain WIN-330-142-01 as a clear oil (169 mg, 75%). ¹H NMR (CDCl₃): δ 7.19-7.35 (m, 2H), 6.68-6.83 (m, 3H), 4.18 (q, J 7.1 Hz, 2H), 3.34-3.46 (m, 2H), 2.97 (s, 3H), 2.32-2.47 (m, 2H), 1.87-2.04 (m, 2H), 1.30 (t, J 7.0 Hz, 3H). MS, m/z=225 (100) [M+H]⁺.

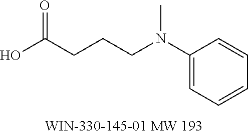

General Procedure B was followed using WIN-330-142-01 (169 mg, 0.76 mmol) to obtain WIN-330-145-01 as a clear oil (144 mg, 98%). ¹H NMR (CDCl₃): δ 7.21-7.33 (m, 2H), 6.70-6.82 (m, 3H), 3.33-3.48 (m, 2H), 2.96 (s, 3H), 2.45 (t, J 7.2 Hz, 2H), 1.96 (quin, J 7.3 Hz, 2H). MS, m/z=194 (100) [M+H]⁺.

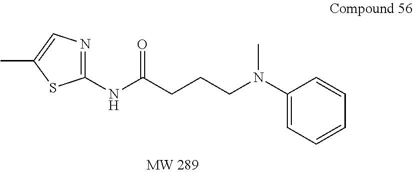 [SMILES: Cc1cnc(NC(=O)CCCN(C)c2ccccc2)s1]

General Procedure C was followed using WIN-330-145-01 (36 mg, 0.19 mmol) and 5-methyl-2-aminothiazole (21 mg, 0.19 mmol) to obtain WIN-330-146-01 as a white solid (36 mg, 67%). ¹H NMR (CDCl₃): δ 7.15-7.30 (m, 2H), 6.96 (d, J 1.3 Hz, 1H), 6.65-6.79 (m, 3H), 3.45 (t, J 6.9 Hz, 2H), 2.94 (s, 3H), 2.59 (t, J 7.3 Hz, 2H), 2.38 (d, J 1.1 Hz, 3H), 2.09 (quin, J 7.1 Hz, 2H). MS, m/z=290 (100) [M+H]⁺.

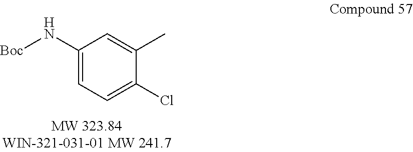 [SMILES: Cc1cc(NC(=O)OC(C)(C)C)ccc1Cl]

4-Chloro-3-methylaniline (1.00 g, 7.06 mmol) was dissolved in 15 ml of DCM and cooled to 0° C. under N₂. Boc anhydride (1.70 g, 7.77 mmol) was then added portion wise to the reaction which was allowed to warm to 20° C. and stirred for 20 h. The organic layer was then washed with water (15 ml), brine (15 ml), dried with Na₂SO₄, filtered and concentrated in vacuo. The crude residue was then purified by column chromatography (100% CyHex to 10% EtOAc/CyHex) to obtain WIN-330-031-01 as white crystals (608 mg, 36%). ¹H NMR (CDCl₃): δ 7.30 (d, J 2.4 Hz, 1H), 7.21 (d, J 8.8 Hz, 1H), 7.05-7.11 (m, 1H), 6.48 (br s, 1H), 2.33 (s, 3H), 1.51 (s, 9H)

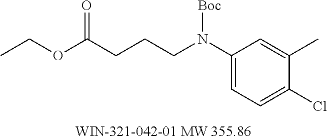 [SMILES: CCOC(=O)CCCN(C(=O)OC(C)(C)C)c1ccc(Cl)c(C)c1]

WIN-321-031-01 (270 mg, 1.12 mmol) was dissolved in anhydrous DMF (4 ml) and cooled to 0° C. under N₂. Sodium hydride (60% in mineral oil) (58 mg, 1.45 mmol) was then added and reaction allowed to warm to 20° C. over 30 min. Ethyl bromobutyrate (178 μl, 1.23 mmol) was then added in 5 portions over 10 min followed by potassium iodide (185 mg, 1.12 mmol). The reaction was stirred at 20° C. for 4 h and then at 60° C. for 14 h. The reaction was then quenched by saturated NH₄Cl. The solvent was then evaporated and the crude residue dissolved in EtOAc (20 ml) which was washed with water (10 ml), brine (10 ml), dried with Na₂SO₄, filtered and concentrated in vacuo. The crude residue was then purified by column chromatography (100% CyHex to 10% EtOAc/CyHex) to obtain WIN-321-042-01 as a clear oil (62 mg, 16%). ¹H NMR (CDCl₃): δ 7.11-7.19 (m, 1H), 7.08 (d, J 2.2 Hz, 1H), 6.98 (dd, J 8.5, 2.5 Hz, 1H), 6.57 (br s, 1H), 3.58-3.72 (m, 2H), 2.29-2.37 (m, 5H), 1.87 (quin, J 7.4 Hz, 2H), 1.45 (s, 9H), 1.25 (t, J 7.2 Hz, 3H). MS, m/z=255 (100) [M−100]

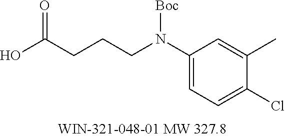 [SMILES: Cc1cc(N(CCCC(=O)O)C(=O)OC(C)(C)C)ccc1Cl]

General Procedure B was followed using WIN-321-042-01 (62 mg, 0.17 mmol) to obtain WIN-321-048-01 as a clear oil (46 mg, 81%). ¹H NMR (CDCl₃): δ 7.30 (d, J 8.6 Hz, 1H), 7.08 (d, J 2.0 Hz, 1H), 6.97 (dd, J 8.5, 2.3 Hz, 1H), 3.62-3.74 (m, 2H), 2.36-2.42 (m, 5H), 1.80-1.93 (m, 2H), 1.44 (s, 9H), MS, m/z=326 (100) [M−H]⁻, 328 (30)

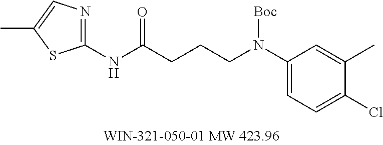 [SMILES: Cc1cnc(NC(=O)CCCN(C(=O)OC(C)(C)C)c2ccc(Cl)c(C)c2)s1]

General Procedure C was followed using WIN-321-048-01 (42 mg, 0.13 mmol) and 5-methyl-2-aminothiazole (18 mg, 0.15 mmol) to obtain WIN-321-050-01 as a white solid (33 mg, 60%). ¹H NMR (d₆-Acetone): δ 7.35 (d, J 8.6 Hz, 1H), 7.30 (d, J 2.6 Hz, 1H), 7.13-7.19 (m, 1H), 7.04 (d, J 1.3 Hz, 1H), 3.72-3.78 (m, 2H), 2.57 (t, J 7.4 Hz, 2H), 2.34-2.38 (m, 6H), 1.89-1.98 (m, 2H). MS, m/z=424 (100) [M+H]+

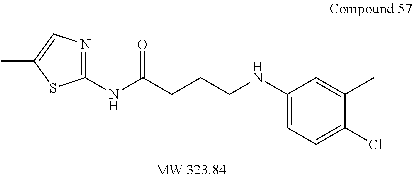 [SMILES: Cc1cnc(NC(=O)CCCNc2ccc(Cl)c(C)c2)s1]

WIN-321-050-01 (33 mg, 0.079 mmol) was dissolved in a 1:3 mixture of TFA/DCM (4 ml) and stirred at 20° C. over 30 min. The solvent was then evaporated in vacuo and the crude residue dissolved in EtOAc (10 ml) which was then washed with NaHCO₃ (10 ml), water (10 ml), brine (10 ml), dried with Na₂SO₄, filtered and concentrated in vacuo to obtain WIN-321-050-02 as a white solid (22 mg, 86%). ¹H NMR (d₆-Acetone): δ 7.02-7.07 (m, 2H), 6.58 (d, J 3.1 Hz, 1H), 6.48 (dd, J 8.7, 3.0 Hz, 1H), 3.16-3.24 (m, 2H), 2.66 (t, J 7.3 Hz, 2H), 2.38 (d, J 1.3 Hz, 3H), 2.24 (s, 3H), 1.96-2.04 (m, 2H). MS, m/z=324 (100) [M+H]⁺, 326 (30)

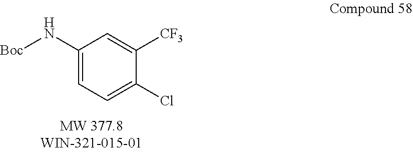 [SMILES: CC(C)(C)OC(=O)Nc1ccc(Cl)c(C(F)(F)F)c1]

The procedure used for WIN-321-031-01 was followed using 4-chloro-3-(trifluoromethyl)aniline (1.00 g, 5.11 mmol) to obtain WIN-321-015 as white crystals (935 mg, 62%). ¹H NMR (CDCl₃): δ 7.75 (d, J 2.6 Hz, 1H), 7.52 (dd, J 8.9, 2.5 Hz, 1H), 7.42 (d, J 8.6 Hz, 1H), 6.60 (br s, 1H), 1.54 (s, 9H)

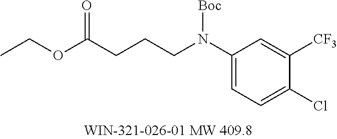 [SMILES: CCOC(=O)CCCN(C(=O)OC(C)(C)C)c1ccc(Cl)c(C(F)(F)F)c1]

The procedure used for WIN-321-042-01 was followed using WIN-321-026-01 (300 mg, 1.01 mmol) to give WIN-321-026-01 as a clear oil (132 mg, 32%). ¹H NMR (CDCl₃): δ 7.57 (d, J 2.6 Hz, 1H), 7.48 (d, J 8.6 Hz, 1H), 7.36 (dd, J 8.6, 2.4 Hz, 1H), 4.13 (q, J 7.3 Hz, 2H), 3.66-3.76 (m, 2H), 2.34 (t, J 7.4 Hz, 2H), 1.77-1.97 (m, 2H), 1.41-1.55 (m, 9H), 1.25 (t, J 7.2 Hz, 3H). MS, m/z=310 (100) [M−100]

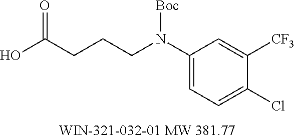 [SMILES: CC(C)(C)OC(=O)N(CCCC(=O)O)c1ccc(Cl)c(C(F)(F)F)c1]

General Procedure B was followed using WIN-321-026-01 (62 mg, 0.17 mmol) to obtain WIN-321-032-01 as a white powder (116 mg, 96%). ¹H NMR (CDCl₃): δ 7.57 (d, J 2.4 Hz, 1H), 7.48 (d, J 8.6 Hz, 1H), 7.35 (dd, J 8.6, 2.2 Hz, 1H), 3.74 (dd, J 7.8, 6.7 Hz, 2H), 2.42 (t, J 7.2 Hz, 2H), 1.90 (quin, J 7.3 Hz, 2H), 1.46 (s, 9H). MS, m/z=382 (100) [M+H]⁺, 384 (30)

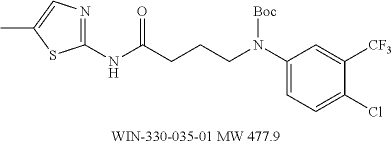 [SMILES: Cc1cnc(NC(=O)CCCN(C(=O)OC(C)(C)C)c2ccc(Cl)c(C(F)(F)F)c2)s1]

General Procedure C was followed using WIN-321-026-01 (116 mg, 0.304 mmol) and 5-methyl-2-aminothiazole (42 mg, 0.365 mmol) to obtain WIN-321-035-01 as a white solid (114 mg, 79%). ¹H NMR (CDCl₃): δ 7.56 (d, J 2.4 Hz, 1H), 7.45 (d, J 8.6 Hz, 1H), 7.35 (d, J 8.4 Hz, 1H), 7.09 (s, 1H), 3.79 (t, J 7.0 Hz, 2H), 2.60 (t, J 7.2 Hz, 2H), 2.43 (s, 3H), 1.96-2.10 (m, 2H), 1.43 (s, 9H). MS, m/z=478 (100) [M+H]⁺

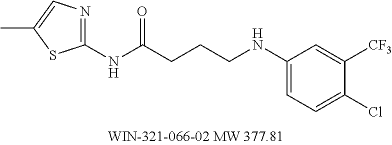 [SMILES: Cc1cnc(NC(=O)CCCNc2ccc(Cl)c(C(F)(F)F)c2)s1]

The procedure used for WIN-321-050-02 was followed using WIN-321-035-01 (110 mg, 0.23 mmol) to obtain WIN-321-066-02 as a white solid (72 mg, 83%). ¹H NMR (d₆-Acetone): δ 7.02-7.08 (m, 2H), 6.58 (d, J 3.1 Hz, 1H), 6.48 (dd, J 8.7, 3.0 Hz, 1H), 3.15-3.26 (m, 2H), 2.66 (t, J 7.3 Hz, 2H), 2.34-2.41 (m, 3H), 2.24 (s, 3H), 1.93-2.04 (m, 2H). MS, m/z=378 (100) [M+H]⁺, 380 (30)

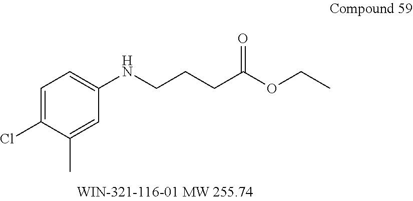 [SMILES: CCOC(=O)CCCNc1ccc(Cl)c(C)c1]

4-Chloro-3-methylaniline (400 mg, 2.82 mmol), ethyl bromobutyrate (408 μl, 2.82 mmol), K₂CO₃ (781 mg, 5.86 mmol) and potassium iodide (469 mg, 42.8 mmol) were dissolved in 30 ml of DMF and stirred at reflux under N₂ for 16 h. The solvent was then evaporated in vacuo and the crude residue dissolved in EtOAc (40 ml) which was washed with water (30 ml), brine (30 ml), dried with Na₂SO₄, filtered and concentrated in vacuo. The crude residue was then purified by column chromatography (100% CyHex to 20% EtOAc/CyHex) to obtain WIN-321-116-01 as a clear oil (220 mg, 30%). ¹H NMR (CDCl₃): δ 7.11 (d, J 8.6 Hz, 1H), 6.48 (d, J 2.9 Hz, 1H), 6.39 (dd, J 8.6, 2.9 Hz, 1H), 4.16 (q, J 7.0 Hz, 2H), 3.15 (t, J 6.9 Hz, 2H), 2.43 (t, J 7.2 Hz, 2H), 2.31 (s, 3H), 1.95 (t, J 7.0 Hz, 2H), 1.28 (t, J 7.2 Hz, 3H). MS, m/z=256.2 (100) [M+H]⁺, 258.0 (30)

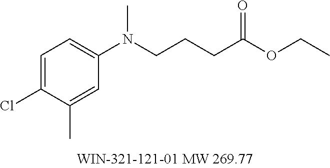 [SMILES: CCOC(=O)CCCN(C)c1ccc(Cl)c(C)c1]

To a solution of WIN-321-116-01 (220 mg, 0.86 mmol) and potassium carbonate (238 mg, 1.72 mmol) in ACN (5 ml) was added iodomethane (107 μl, 1.72 mmol) which was then stirred at reflux for 16 h. The solvent was then evaporated in vacuo and the crude residue dissolved in EtOAc (20 ml) which was washed with water (15 ml), brine (15 ml), dried with Na₂SO₄, filtered and concentrated in vacuo. The crude residue was then purified by column chromatography (100% CyHex to 10% EtOAc/CyHex) to obtain WIN-321-121-01 as a clear oil (140 mg, 60%). ¹H NMR (CDCl₃): δ 7.16 (d, J 8.6 Hz, 1H), 6.57 (br s, 1H), 6.51 (d, J 8.1 Hz, 1H), 4.15 (q, J 7.1 Hz, 2H), 3.28-3.39 (m, 2H), 2.92 (s, 3H), 2.30-2.38 (m, 5H), 1.93-1.88 (m, 2H), 1.27 (t, J 7.2 Hz, 3H). MS, m/z=270 (100) [M+H]⁺, 272 (30)

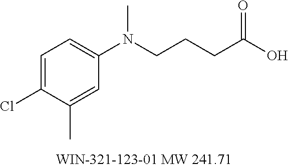 [SMILES: Cc1cc(N(C)CCCC(=O)O)ccc1Cl]

General Procedure B was followed using WIN-321-121-01 (140 mg, 0.52 mmol) to obtain WIN-321-123-01 as a yellow oil (110 mg, 88%). ¹H NMR (CDCl₃): δ 7.17 (d, J 8.8 Hz, 1H), 6.60 (d, J 3.1 Hz, 1H), 6.52 (dd, J 8.8, 3.1 Hz, 1H), 3.32-3.39 (m, 2H), 2.92 (s, 3H), 2.43 (t, J 7.2 Hz, 2H), 2.34 (s, 3H), 1.86-1.99 (m, 2H). MS, m/z=240 (100) [M−H]⁻, 242 (30)

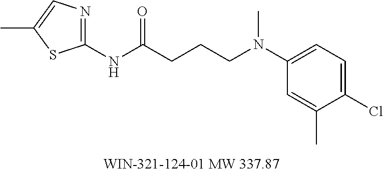 [SMILES: Cc1cnc(NC(=O)CCCN(C)c2ccc(Cl)c(C)c2)s1]

General Procedure C was followed using WIN-321-123-01 (36 mg, 0.15 mmol) and 5-methyl-2-aminothiazole (20 mg, 0.18 mmol) to obtain WIN-321-035-01 as a white solid (33 mg, 65%). ¹H NMR (CDCl₃): δ 7.18 (d, J 8.6 Hz, 1H), 7.02 (d, J 1.3 Hz, 1H), 6.65 (d, J 2.9 Hz, 1H), 6.57 (dd, J 8.7, 2.97 Hz, 1H), 3.49 (t, J 6.9 Hz, 2H), 2.97 (s, 3H), 2.63 (t, J 7.2 Hz, 2H), 2.46 (d, J 1.1 Hz, 3H), 2.35 (s, 3H), 2.14 (t, J 6.9 Hz, 2H). MS, m/z=338 (100) [M+H]⁺, 340 (30)

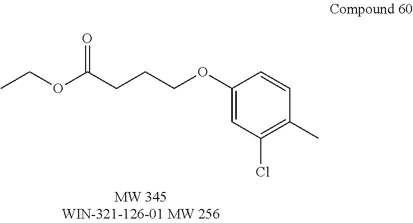 [SMILES: CCOC(=O)CCCOc1ccc(C)c(Cl)c1]

General Procedure A was followed using 3-chloro-4-methylphenol (1.0 g, 7.01 mmol) and ethyl bromobutyrate (1.21 ml, 8.42 mmol) to obtain WIN-321-126-01 as a clear oil (1.72 mg, 96%). ¹H NMR (CDCl₃): δ 7.07-7.16 (m, 1H), 6.91 (d, J 2.4 Hz, 1H), 6.72 (dd, J 8.5, 2.5 Hz, 1H), 4.17 (q, J 7.3 Hz, 2H), 3.99 (t, J 7.3 Hz, 2H), 2.50 (m, 2H), 2.31 (s, 3H), 2.03-2.19 (m, 2H), 1.28 (t, J 7.2 Hz, 3H). MS, m/z=257 (100) [M+H]⁺, 259 (30).

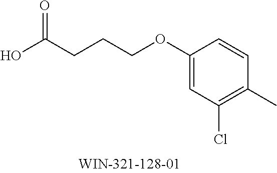

General Procedure B was followed using WIN-321-126-01 (1.70 g, 6.62 mmol) to obtain WIN-321-128-01 as a white solid (1.42 g, 94%). ¹H NMR (CDCl₃): δ 7.12 (dd, J 8.4, 0.44 Hz, 1H), 6.92 (d, J 2.6 Hz, 1H), 6.72 (dd, J 8.5, 2.75 Hz, 1H), 4.00 (t, J 6.1 Hz, 2H), 2.60 (t, J 7.3 Hz, 2H), 2.31 (s, 3H), 2.06-2.20 (m, 2H). MS, m/z=226 (100) [M−H]⁻.

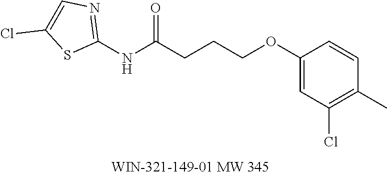 [SMILES: Cc1ccc(OCCCC(=O)Nc2ncc(Cl)s2)cc1Cl]

General Procedure C was followed using WIN-321-126-01 (50 mg, 0.22 mmol) and 5-chlorothiazol-2-amine HCl (45 mg, 0.26 mmol) to obtain WIN-321-149-01 as a white solid (14 mg, 19%). ¹H NMR (CDCl₃): δ 7.33 (s, 1H), 7.01-7.16 (m, 1H), 6.91 (d, J 2.6 Hz, 1H), 6.71 (dd, J 8.3, 2.5 Hz, 1H), 4.05 (t, J 5.7 Hz, 2H), 2.73 (t, J 7.3 Hz, 2H), 2.21-2.32 (m, 5H). MS, m/z=345 (100) [M+H]⁺, 347 (70).

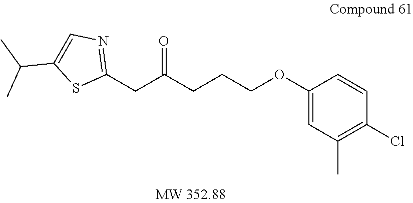 [SMILES: Cc1cc(OCCCC(=O)Cc2ncc(C(C)C)s2)ccc1Cl]

General Procedure C was followed using BES-AA0-986-B1 (50 mg, 0.22 mmol) and WIN-321-081-01 (37 mg, 0.26 mmol) to obtain WIN-321-083-02 as a white solid (34 mg, 44%). ¹H NMR (CDCl₃): δ 7.23-7.33 (m, 1H), 7.14 (d, J 0.9 Hz, 1H), 6.85-7.00 (m, 2H), 4.05-4.13 (m, 2H), 3.15 (dd, J 7.3, 6.4 Hz, 1H), 2.81 (t, J 7.3 Hz, 2H), 2.21-2.36 (m, 2H), 1.32-1.37 (t, J 7.3 Hz, 6H) MS, m/z=353 (100) [M+H]⁺, 355 (30).

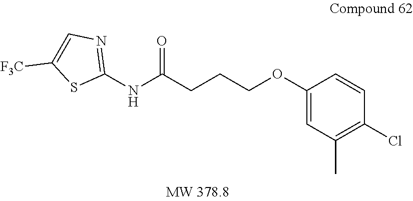 [SMILES: Cc1cc(OCCCC(=O)Nc2ncc(C(F)(F)F)s2)ccc1Cl]

General Procedure C was followed using BES-AA0-986-B1 (33 mg, 0.14 mmol) and 5-(trifluoromethyl)thiazol-2-amine (20 mg, 0.12 mmol) to obtain WIN-321-098-01 as a white solid (16 mg, 36%). ¹H NMR (CDCl₃): δ 7.82 (s, 1H), 7.22 (d, J 8.8 Hz, 1H), 6.75 (d, J 3.1 Hz, 1H), 6.65 (dd, J 8.8, 3.1 Hz, 1H), 4.06 (t, J 5.7 Hz, 2H), 2.79 (t, J 7.0 Hz, 2H), 2.23-2.37 (m, 5H). MS, m/z=379 (100) [M+H]⁺, 381 (30).

Piperazines

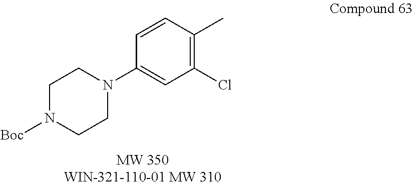 [SMILES: Cc1ccc(N2CCN(C(=O)OC(C)(C)C)CC2)cc1Cl]

2-Chloro-4-iodotoluene (250 μl, 1.78 mmol), 1-Boc-piperazine (398 mg, 2.14 mmol), Pd₂(dba)₃ (40.8 mg, 0.045 mmol), Xantphos (103 mg, 0.178 mmol) and potassium tert butoxide (280 mg, 2.50 mmol) were dissolved in anhydrous toluene (5 ml) and heated at reflux for 16 h under N₂. The reaction was then concentrated and dissolved in EtOAc (20 ml), filtered through Celite and washed with additional EtOAc (50 ml). The organic layer was then washed with water (2×20 ml), brine (2×20 ml), dried with Na₂SO₄, filtered and concentrated in vacuo. The crude residue was then purified by column chromatography (100% CyHex to 10% EtOAc/CyHex) to obtain WIN-321-110-01 as an oil (436 mg, 79%). ¹H NMR (CDCl₃): δ 7.11 (d, J 8.4 Hz, 1H), 6.92 (d, J 2.4 Hz, 1H), 6.75 (dd, J 8.6, 2.6 Hz, 1H), 3.69-3.51 (m, 4H), 3.18-3.04 (m, 4H), 2.29 (s, 3H), 1.53-1.45 (s, 9H). MS, m/z=311 (100) [M+H]⁺, 313 (30).

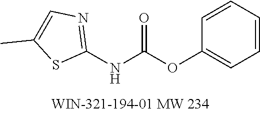 [SMILES: Cc1cnc(NC(=O)Oc2ccccc2)s1]

5-Methyl-2-aminothiazole (1.5 g, 13.1 mmol) was dissolved in pyridine (8 ml) and cooled to 0° C. under N₂. Phenyl chloroformate (3.62 ml, 28.9 mmol) was then added dropwise and reaction stirred for 5 h at this temperature. The reaction was then quenched with water (10 ml) and the resulting precipitate filtered. The crude precipitate was then purified by column chromatography (100% DCM) to obtain WIN-321-194-01 as a white solid (590 mg, 19%). ¹H NMR (CDCl₃): δ 7.48-7.36 (m, 2H), 7.31-7.25 (m, 2H), 7.24-7.22 (m, 1H), 7.10 (d, J 1.3 Hz, 1H), 2.37 (d, J 1.1 Hz, 3H). MS, m/z=235 [M+H]⁺.

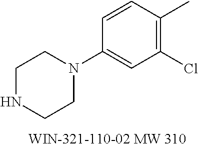

WIN-321-010-01 (436 mg, 1.40 mmol) was dissolved in a 1:3 mixture of TFA/DCM (10 ml) and stirred at 20° C. over 1 h. The solvent was then evaporated in vacuo and the crude residue dissolved in EtOAc (30 ml) which was then washed with NaHCO₃ (20 ml), water (20 ml), brine (20 ml), dried with Na₂SO₄, filtered and concentrated in vacuo to obtain WIN-321-010-02 as a solid (288 mg, 97%). ¹H NMR (CDCl₃): δ 7.10 (dd, J 8.5, 0.6 Hz, 1H), 6.91 (d, J 2.6 Hz, 1H), 6.74 (dd, J 8.5, 2.6 Hz, 1H), 3.23-2.99 (m, 8H), 2.29 (s, 3H). MS, m/z=211 (100) [M+H]⁺, 213 (30).

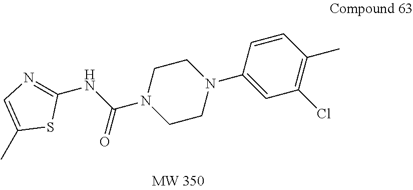

WIN-321-110-02 (26 mg, 0.12 mmol), WIN-321-194-01 (31.8 mg, 0.14 mmol) and caesium carbonate (80 mg, 0.25 mmol) were combined in dioxane (1 ml) and stirred at reflux for 5 h. The reaction was then cooled to room temperature and the reaction mixture diluted with EtOAc (20 ml) which was then washed with water (10 ml), brine (10 ml), dried with Na₂SO₄, filtered and concentrated in vacuo. The crude residue was then purified by column chromatography (100% CyHex to 50% EtOAc/CyHex) to obtain WEHI-1250190 as a white solid (13 mg, 30%). ¹H NMR (CDCl₃): δ 7.12 (dd, J 8.4, 0.7 Hz, 1H), 6.97-6.90 (m, 2H), 6.77-6.70 (m, 1H), 3.77-3.63 (m, 4H), 3.27-3.11 (m, 4H), 2.37 (d, J 1.1 Hz, 3H), 2.29 (s, 3H). MS, m/z=351 (100) [M+H]⁺, 353 (30)

Carbamate Intermediates

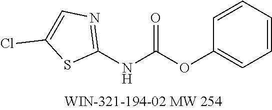 [SMILES: O=C(Nc1ncc(Cl)s1)Oc1ccccc1]

The procedure used for WIN-321-194-01 was followed using 2-amino-5-chlorothiazole hydrochloride (700 mg, 4.09 mmol) and phenyl chloroformate (1.13 ml, 9.00 mmol) to give WIN-321-194-02 (518 mg, 50%) as a white solid. ¹H NMR (CDCl₃): (7.49-7.41 (m, 2H), 7.35-7.28 (m, 2H), 7.26-7.22 (m, 2H). MS, m/z=255 (100) [M+H]⁺, 257 (60)

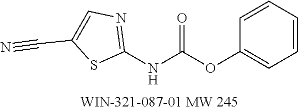 [SMILES: N#Cc1cnc(NC(=O)Oc2ccccc2)s1]

The procedure used for WIN-321-194-01 was followed using 2-aminothiazole-5-carbonitrile (240 mg, 1.92 mmol) and phenyl chloroformate (0.48 ml, 3.84 mmol) to give WIN-321-087-01 (230 mg, 49%) as a solid. ¹H NMR (CDCl₃): δ 7.98 (s, 1H), 7.48-7.43 (m, 2H), 7.37-7.29 (m, 2H), 7.26-7.22 (m, 1H). MS, m/z=246 (100) [M+H]⁺.

The below examples were generated following similar methods to those outlined above.

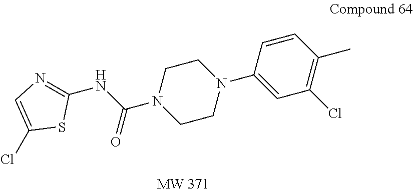 [SMILES: Cc1ccc(N2CCN(C(=O)Nc3ncc(Cl)s3)CC2)cc1Cl]

¹H NMR (CDCl₃): δ 7.17-7.08 (m, 2H), 6.98-6.89 (m, 1H), 6.75 (dd, J 8.4, 2.6 Hz, 1H), 3.79-3.59 (m, 4H), 3.31-3.06 (m, 4H), 2.30 (s, 3H). MS, m/z=371 (100) [M+H]⁺, 373 (60).

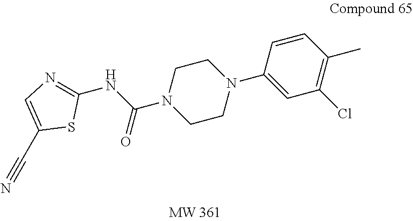 [SMILES: Cc1ccc(N2CCN(C(=O)Nc3ncc(C#N)s3)CC2)cc1Cl]

1H NMR (d₆-Acetone): δ 8.06 (s, 1H), 7.19 (d, J 8.8 Hz, 1H), 7.02 (d, J 2.6 Hz, 1H), 6.91 (dd, J 8.5, 2.8 Hz, 1H), 3.88-3.78 (m, 4H), 3.33-3.22 (m, 4H), 2.26 (s, 3H). MS, m/z=362 (100) [M+H]⁺, 364 (30).

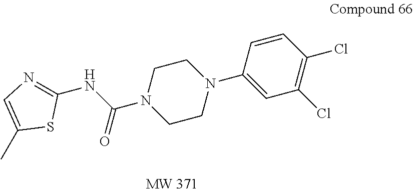 [SMILES: Cc1cnc(NC(=O)N2CCN(c3ccc(Cl)c(Cl)c3)CC2)s1]

¹H NMR (CDCl₃): δ 7.33 (d, J 8.8 Hz, 1H), 6.99 (d, J 2.6 Hz, 2H), 6.77 (dd, J 9.0, 2.9 Hz, 1H), 3.80-3.72 (m, 4H), 3.28-3.20 (m, 4H), 2.39 (s, 3H). MS, m/z=371 (100) [M+H]⁺, 373 (60).

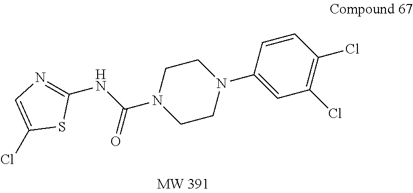 [SMILES: O=C(Nc1ncc(Cl)s1)N1CCN(c2ccc(Cl)c(Cl)c2)CC1]

¹H NMR (CDCl₃): δ 7.33 (d, J 9.0 Hz, 1H), 7.19 (s, 1H), 6.99 (d, J 2.9 Hz, 1H), 6.77 (dd, J 8.9, 3.0 Hz, 1H), 3.76-3.68 (m, 4H), 3.30-3.20 (m, 4H). MS, m/z=391 (100) [M+H]⁺, 393 (90).

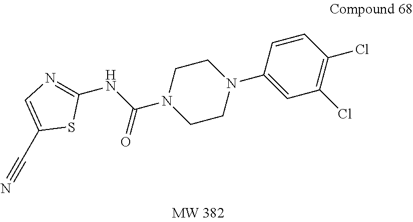 [SMILES: N#Cc1cnc(NC(=O)N2CCN(c3ccc(Cl)c(Cl)c3)CC2)s1]

¹H NMR (CDCl₃): δ 7.94 (s, 1H), 7.37-7.30 (m, 1H), 7.00 (d, J 2.86 Hz, 1H), 6.78 (dd, J 8.9, 2.8 Hz, 1H), 3.79-3.70 (m, 4H), 3.32-3.23 (m, 4H). MS, m/z=382 (100) [M+H]⁺, 384 (60).

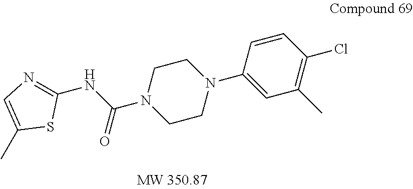 [SMILES: Cc1cnc(NC(=O)N2CCN(c3ccc(Cl)c(C)c3)CC2)s1]

¹H NMR (CDCl₃): δ 7.23 (d, J 8.6 Hz, 1H), 6.94 (d, J 1.3 Hz, 1H), 6.80 (d, J 2.9 Hz, 1H), 6.71 (dd, J 8.7, 3.0 Hz, 1H), 3.78-3.64 (m, 4H), 3.24-3.11 (m, 4H), 2.41-2.33 (m, 6H), MS, m/z=351 (100) [M+H]⁺, 353 (30).

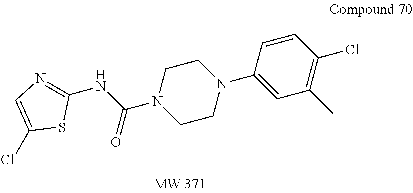 [SMILES: Cc1cc(N2CCN(C(=O)Nc3ncc(Cl)s3)CC2)ccc1Cl]

¹H NMR (CDCl₃): δ ppm 7.23 (d, J 8.80 Hz, 1H), 7.15 (s, 1H), 6.80 (d, J 2.86 Hz, 1H), 6.70 (dd, J 8.69, 2.97 Hz, 1H), 3.75-3.63 (m, 4H), 3.23-3.12 (m, 4H), 2.35 (s, 3H). MS, m/z=371 (100) [M+H]⁺, 373 (60).

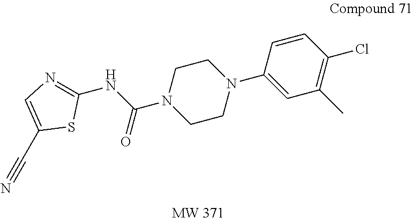 [SMILES: Cc1cc(N2CCN(C(=O)Nc3ncc(C#N)s3)CC2)ccc1Cl]

¹H NMR (CDCl₃): δ 7.93 (br. s., 1H), 7.52-7.41 (m, 1H), 6.95-6.88 (m, 1H), 6.82 (d, J 8.6 Hz, 1H), 3.88-3.72 (m, 4H), 3.35-3.20 (m, 4H), 2.38 (s, 3H). MS, m/z=362 (100) [M+H]⁺, 364 (30).

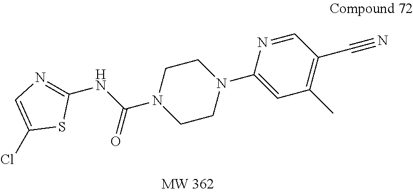 [SMILES: Cc1cc(N2CCN(C(=O)Nc3ncc(Cl)s3)CC2)ncc1C#N]

¹H NMR (CDCl₃): δ 8.36 (s, 1H), 6.95 (s, 1H), 6.47 (s, 1H), 3.85-3.76 (m, 4H), 3.76-3.62 (m, 4H), 2.45 (s, 3H). MS, m/z=363 (100) [M+H]⁺, 365 (30).

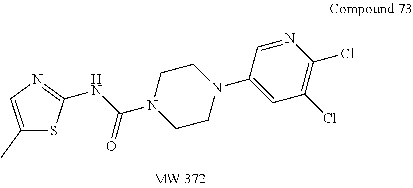 [SMILES: Cc1cnc(NC(=O)N2CCN(c3cnc(Cl)c(Cl)c3)CC2)s1]

¹H NMR (CDCl₃): δ 7.98 (d, J 2.9 Hz, 1H), 7.30 (d, J 2.9 Hz, 1H), 6.93 (d, J 1.3 Hz, 1H), 3.81-3.72 (m, 4H), 3.33-3.23 (m, 4H), 2.38 (d, J 1.1 Hz, 3H). MS, m/z=372 (100) [M+H]⁺, 374 (60).

Homopiperazines

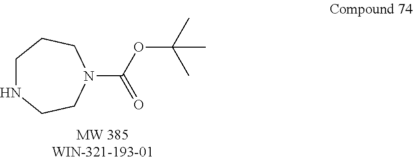

Homopiperazine (5.00 g, 49.92 mmol) was dissolved in methanol (200 ml) and cooled to 0° C. Boc anhydride (12 g, 55.0 mmol) in methanol (100 ml) was added dropwise over 1 h and the reaction allowed to warm to room temperature after which the reaction was heated to reflux for 4 h. The reaction was concentrated in vacuo and dissolved in 1 M citric acid (150 ml). The aqueous layer was then washed with EtOAc (3×70 ml). The aqueous layer was then cooled to 0° C. made basic with solid Na₂CO₃. The product was then extracted with EtOAc (3×100 ml), dried with Na₂SO₄, filtered and concentrated in vacuo to give WIN-321-193-01 as a clear oil (1.08 g, 11% yield). ¹H NMR (CDCl₃): δ 3.54-3.37 (m, 4H), 2.96-2.81 (m, 4H), 1.87 (br. s., 1H), 1.84-1.72 (m, 2H), 1.47 (s, 9H).

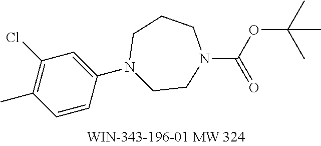 [SMILES: Cc1ccc(N2CCCN(C(=O)OC(C)(C)C)CC2)cc1Cl]

The procedure used for WIN-321-110-01 was followed using 2-Chloro-4-iodotoluene (139 μl, 0.99 mmol), WIN-321-193-01 (198 mg, 0.99 mmol) to give WIN-343-196-01 as a clear oil (130 mg, 40%). ¹H NMR (CDCl₃): δ 7.04 (d, J 8.6 Hz, 1H), 6.70 (s, 1H), 6.53 (d, J 8.14 Hz, 1H), 3.64-3.41 (m, 6H), 3.38-3.17 (m, 2H), 2.25 (s, 3H), 1.98 (quin, J 5.9 Hz, 2H), 1.48-1.33 (ad, J 20 Hz, 9H). MS, m/z=325 (100) [M+H]⁺, 327 (30).

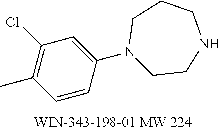

The procedure used for WIN-321-110-02 was followed using WIN-343-196-01 (130 mg, 0.40 mmol) to give WIN-343-198-01 as a solid (71 mg, 79%). ¹H NMR (CDCl₃): δ 7.04 (dd, J 8.47, 0.55 Hz, 1H), 6.69 (d, J 2.6 Hz, 1H), 6.51 (dd, J 8.6, 2.6 Hz, 1H), 3.54 (t, J 6.1 Hz, 4H), 3.10-3.00 (m, 2H), 2.93-2.84 (m, 2H), 2.26 (s, 3H), 2.04-1.90 (m, 2H). MS, m/z=225 (100) [M+H]⁺, 227 (30).

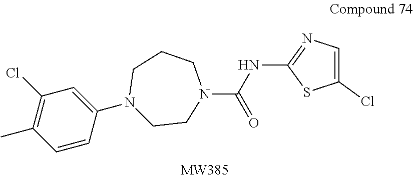 [SMILES: Cc1ccc(N2CCCN(C(=O)Nc3ncc(Cl)s3)CC2)cc1Cl ; "MW385"]

The procedure used for WIN-321-208-03 was followed using WIN-343-198-01 (24 mg, 0.11 mmol) and WIN-321-194-02 (27 mg, 0.11 mmol) to obtain WIN-321-208-03 as a white solid (16 mg, 39%). ¹H NMR (CDCl₃): δ 7.98 (d, J 2.9 Hz, 1H), 7.30 (d, J 2.9 Hz, 1H), 6.93 (d, J 1.3 Hz, 1H), 3.82-3.72 (m, 4H), 3.33-3.22 (m, 4H), 2.38 (d, J 1.1 Hz, 3H). MS, m/z=385 (100) [M+H]⁺, 387 (60).

The example below was generated following similar methods to those outlined above.

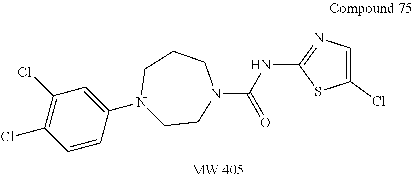 [SMILES: O=C(Nc1ncc(Cl)s1)N1CCCN(c2ccc(Cl)c(Cl)c2)CC1 ; "MW 405"]

¹H NMR (CDCl₃): δ 7.24 (d, J 9.0 Hz, 1H), 7.16 (s, 1H), 6.75 (d, J 3.1 Hz, 1H), 6.54 (dd, J 9.0, 3.1 Hz, 1H), 3.77-3.67 (m, 2H), 3.53-3.66 (m, 4H), 3.43 (t, J 6.2 Hz, 2H), 2.13-2.01 (m, 2H). MS, m/z=405 (100) [M+H]⁺, 407 (90)

Amidothiazole Isosteres

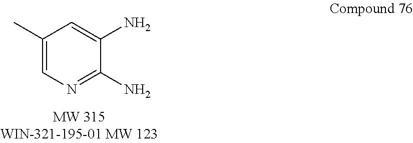

Pd/C (50 mg, 0.47 mmol) was added to a stirred solution of 2-amino-5-methyl-3-nitropyridine (500 mg, 3.27 mmol) was in MeOH (7 ml). The reaction was then evacuated of air 3 times and filled with H₂ gas. The reaction was then stirred under this atmosphere at 20° C. for 5 h after which the reaction was filtered through Celite and washed with further MeOH (30 ml). The solution was concentrated in vacuo to give WIN-321-195-01 (395 mg, 98%). ¹H NMR (MeOD): δ 7.22 (dd, J 2.0, 0.9 Hz, 1H), 6.81 (dd, J 2.0, 0.7 Hz, 1H), 2.13 (t, J 0.7 Hz, 3H). MS, m/z=124 (100).

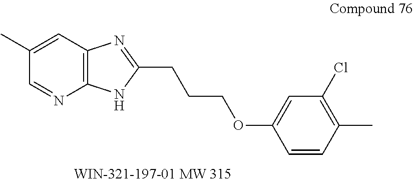

WIN-321-195-01 (120 mg, 0.97 mmol) and WIN-321-128-01 (245 mg, 1.07 mmol) were dissolved in POCl₃ (5 ml) and stirred at reflux for 16 h. The reaction was then cooled to 0° C. and the mixture basified to pH 8 with saturated NaHCO₃. The crude product was extracted with EtOAc (3×15 ml). The organic layers were combined and washed with water (2×20 ml), brine (2×20 ml), dried with anhydrous Na₂SO₄ and filtered. The organic layer was then concentrated to 5 ml after which a precipitate formed. The precipitate was then filtered, washed with water and dried in vacuo to give WIN-321-197-01 as a white solid (103 mg, 33%). 1H NMR (d₆-DMSO): δ 8.16 (d, J 2.0 Hz, 1H), 7.76 (s, 1H), 7.23 (d, J 8.4 Hz, 1H), 6.95 (d, J 2.4 Hz, 1H), 6.80 (dd, J 8.4, 2.4 Hz, 1H), 4.06 (t, J 6.2 Hz, 2H), 3.06-2.95 (m, 2H), 2.41 (s, 3H), 2.30-2.16 (m, 5H). MS, m/z=316 (100) [M+H]⁺, 318 (90).

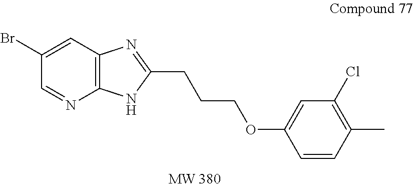 [SMILES: Cc1ccc(OCCCc2nc3cc(Br)cnc3[nH]2)cc1Cl]

The procedure used for WIN-321-197-01 was followed using 2,3-diamino-5-bromopyridine (120 mg, 0.64 mmol) and WIN-321-128-01 (160 mg, 0.70 mmol) to give WIN-321-188-01 as a white solid (50 mg, 21%). 1H NMR (d₆-DMSO): δ 8.52 (d, J 2.2 Hz, 1H), 8.37 (d, J 2.0 Hz, 1H), 7.21 (d, J 9.0 Hz, 1H), 6.88 (d, J 2.6 Hz, 1H), 6.74 (dd, J 8.47, 2.5 Hz, 1H), 4.07 (t, J 6.2 Hz, 2H), 3.13 (t, J 7.3 Hz, 2H), 2.34-2.14 (m, 5H). MS, m/z=380 (100) [M+H]⁺, 382 (100).

Piperidines

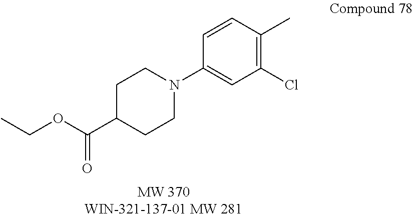 [SMILES: CCOC(=O)C1CCN(c2ccc(C)c(Cl)c2)CC1]

The procedure used for WIN-321-110-01 was followed using ethyl isonipecotate (470 μl, 3.05 mmol) and 2-chloro-4-iodotoluene (389 μl, 2.77 mmol) to give WIN-321-137-01 as an oil (130 mg, 17%). ¹H NMR (CDCl₃): δ 7.09 (d, J 8.36 Hz, 1H), 6.92 (br. s., 1H), 6.82-6.66 (m, 1H), 4.17 (q, J 7.0 Hz, 2H), 3.58 (dt, J 12.5, 3.4 Hz, 2H), 2.77 (t, J 12.1 Hz, 2H), 2.53-2.36 (m, 1H), 2.28 (s, 3H), 2.09-1.88 (m, 3H), 1.88-1.77 (m, 1H), 1.28 (t, J 7.15 Hz, 3H). MS, m/z=382 (100) [M+H]⁺, 384 (30).

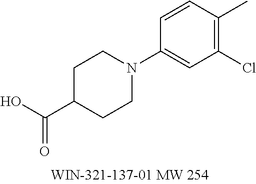 [SMILES: Cc1ccc(N2CCC(C(=O)O)CC2)cc1Cl]

General Procedure B was followed using WIN-321-137-01 (130 mg, 0.46 mmol) to obtain WIN-321-137-02 as a solid (100 mg, 85%). ¹H NMR (CDCl₃): δ 7.10 (d, J 8.14 Hz, 1H), 6.99-6.87 (m, 1H), 6.84-6.68 (m, 1H), 3.60 (dt, J 12.7, 3.36 Hz, 2H), 2.90-2.72 (m, 2H), 2.63-2.42 (m, 1H), 2.30 (s, 3H), 2.15-2.01 (m, 2H), 2.01-1.77 (m, 2H). MS, m/z=252 (100) [M−H]⁻, 254 (30).

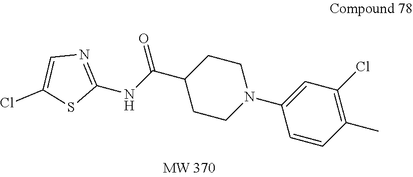 [SMILES: Cc1ccc(N2CCC(C(=O)Nc3ncc(Cl)s3)CC2)cc1Cl]

General Procedure C was followed using WIN-321-137-02 (33 mg, 0.13 mmol) and 5-chlorothiazol-2-amine HCl (27 mg, 0.16 mmol) to obtain WIN-321-139-02 as a white solid (29 mg, 60%). ¹H NMR (CDCl₃): δ 10.45 (s, 1H), 7.12 (d, J 8.80 Hz, 1H), 6.96 (s, 1H), 6.85-6.76 (m, 1H), 3.78-3.64 (m, 2H), 2.88-2.72 (m, 2H), 2.63-2.55 (m, 1H), 2.30 (s, 3H), 2.12-1.98 (m, 4H). MS, m/z=370 (100) [M+H]⁺, 372 (60).

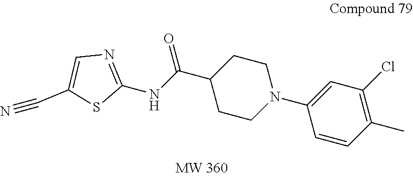 [SMILES: Cc1ccc(N2CCC(C(=O)Nc3ncc(C#N)s3)CC2)cc1Cl]

General Procedure C was followed using WIN-321-137-02 (33 mg, 0.13 mmol) and 2-aminothiazole-5-carbonitrile (20 mg, 0.16 mmol) to obtain WIN-321-139-03 as a white solid (11 mg, 23%). ¹H NMR (CDCl₃): δ 9.61 (s, 1H), 7.97 (s, 1H), 7.13 (dt, J 7.92, 2.53 Hz, 1H), 7.02-6.93 (m, 1H), 6.89-6.74 (m, 1H), 3.78-3.64 (m, 2H), 2.95-2.72 (m, 2H), 2.68-2.51 (m, 1H), 2.30 (s, 3H), 2.22-1.90 (m, 4H). MS, m/z=361 (100) [M+H]⁺, 363 (30).

Pyrrolidines

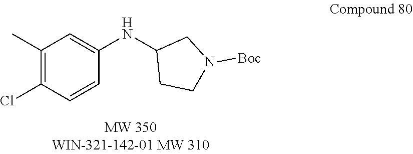 [SMILES: Cc1cc(NC2CCN(C(=O)OC(C)(C)C)C2)ccc1Cl]

Nitrogen gas was purged through a stirred solution of 5-bromo-2-chlorotoluene (452 μl, 3.41 mmol) in 1,4-dioxane (15 ml) for 30 mins. 2,2′-Bis(diphenylphosphino)-1,1′-binaphthyl (212 mg, 0.34 mmol), Pd(OAc)₂ (153 mg, 0.68 mmol) and CS₂CO₃ (2.22 g, 6.81 mmol) were then added. The stirred solution was then purged of nitrogen for a further 30 min after which 1-Boc-3-aminopyrrolidine (745 μl, 4.09 mmol) was added and solution stirred at reflux for 48 h under N₂. The solvent was then evaporated in vacuo and the crude residue dissolved in EtOAc (40 ml) which was then washed with water (2 5×30 ml), brine (2×30 ml), dried with Na₂SO₄, filtered and concentrated. The crude residue was then purified by column chromatography (100% CyHex to 10% EtOAc/CyHex) to obtain WIN-321-142-01 as a solid (111 mg, 10.4%). ¹H NMR (CDCl₃): δ 7.13 (d, J 8.4 Hz, 1H), 6.48 (d, J 2.6 Hz, 1H), 6.39 (dd, J 8.58, 2.86 Hz, 1H), 3.99 (br. s., 1H), 3.79-3.57 (m, 2H), 3.56-3.38 (m, 2H), 3.31-3.14 (m, 1H), 2.30 (s, 3H), 2.10-2.26 (m, 1H), 1.96-1.79 (m, 1H), 1.47 (s, 9H). MS, m/z=255 (100) [M−56], 257 (30).

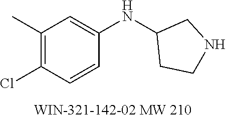

The procedure used for WIN-321-110-02 was followed using WIN-321-142-01 (50 mg, 0.161 mmol) to give WIN-321-142-02 as an oil (32 mg, 94%). ¹H NMR (CDCl₃): δ 7.11 (d, J 8.6 Hz, 1H), 6.47 (d, J 2.6 Hz, 1H), 6.38 (dd, J 8.6, 2.9 Hz, 1H), 3.92 (br. s., 1H), 3.61-2.54 (m, 4H), 2.30 (s, 3H), 2.27-2.04 (m, 3H), 1.75-1.57 (m, 1H). MS, m/z=211 (100) [M+H]⁺, 213 (30).

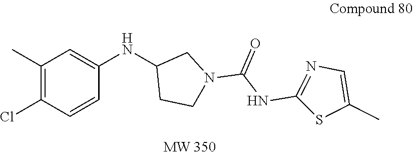 [SMILES: Cc1cnc(NC(=O)N2CCC(Nc3ccc(Cl)c(C)c3)C2)s1]

The procedure used for WIN-321-114-01 was followed using WIN-321-142-02 (16 mg, 0.076 mmol) to give WIN-321-147-02 as a white solid (13 mg, 49%). ¹H NMR (CDCl₃): δ 7.14 (d, J 8.6 Hz, 1H), 6.92 (s, 1H), 6.47 (d, J 2.6 Hz, 1H), 6.38 (dd, J 8.6, 2.9 Hz, 1H), 4.12-4.05 (m, 1H), 3.82-3.74 (m, 1H), 3.66-3.52 (m, 2H), 3.44-3.34 (m, 1H), 2.39-2.18 (m, 7H), 2.05-1.95 (m, 1H). MS, m/z=351 (100) [M+H]⁺, 353 (30).

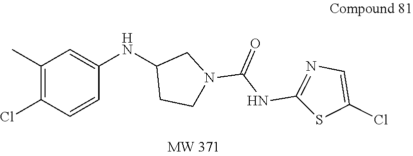 [SMILES: Cc1cc(NC2CCN(C(=O)Nc3ncc(Cl)s3)C2)ccc1Cl]

The procedure used for WIN-321-114-01 was followed using WIN-321-142-02 (16 mg, 0.076 mmol) and WIN-321-194-02 (20 mg, 0.76 mmol) to give WIN-321-149-02 as an oil (12 mg, 43%). ¹H NMR (CDCl₃): δ 7.14 (d, J 8.6 Hz, 2H), 6.46 (d, J 2.6 Hz, 1H), 6.37 (dd, J 8.5, 3.0 Hz, 1H), 4.14-4.06 (m, 1H), 3.83-3.69 (m, 1H), 3.65-3.50 (m, 2H), 3.44-3.34 (m, 1H), 2.39-2.21 (m, 4H), 2.09-1.93 (m, 1H). MS, m/z=371 (100) [M+H]⁺, 373 (30).

Additional Compounds

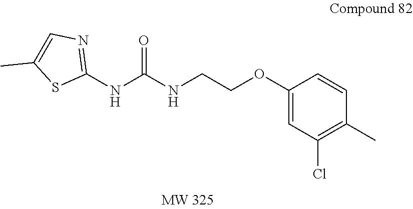

¹H NMR (d₆-Acetone): 67.54 (br. s., 1H), 7.18-7.24 (m, 1H), 7.15 (s, 1H), 6.97-7.03 (m, 1H), 6.87 (dd, J 8.4, 2.6 Hz, 1H), 4.14 (t, J 5.5 Hz, 2H), 3.67 (q, J 5.5 Hz, 2H), 2.38 (d, J 1.3 Hz, 3H), 2.27 (s, 3H). MS, m/z=326 (100) [M+H]⁺, 328 (30).

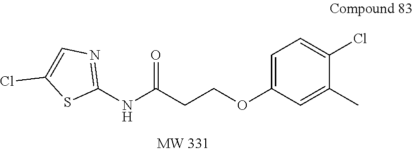 [SMILES: Cc1cc(OCCC(=O)Nc2ncc(Cl)s2)ccc1Cl]

¹H NMR (CDCl₃): δ 7.28 (s, 1H), 7.24 (d, J 8.80 Hz, 1H), 6.81 (d, J 2.64 Hz, 1H), 6.70 (dd, J 8.7, 3.0 Hz, 1H), 4.34 (t, J 5.8 Hz, 2H), 2.94 (t, J 5.8 Hz, 2H), 2.34 (s, 3H). MS, m/z=331 (100) [M+H]⁺, 333 (30).

Biological Assay—FlipIn-FM and FlipIn-RV Dual Cell Reporter Lines

In long-lived latently infected cells, HIV is predominantly integrated into the non-coding introns of transcriptionally active host genes. Transcription of pre-mRNA from the strong upstream cellular promoter reads through the HIV provirus within these introns. Alternative RNA splicing of these read-through cell-HIV pre-mRNAs can cause RNA splicing to the HIV splice sites leading to the formation of chimeric cell-tat mRNAs that translate low levels of Tat protein using internal ribosome entry site (IRES)-mediated translation. Tat is the master regulator for HIV gene expression and is key in driving productive viral infection. Latently infected cells express sub-optimal Tat through an IRES-mediated expression at a level below that required for active and efficient HIV production.

A dual Luciferase reporter cell line (HEK293.IRES-Tat/CMV-CBG/LTR-CBR) that responds to ˜175 μM of transfected Tat protein was used to identify compounds of the invention that specifically induce HIV gene expression in cells with latent HIV. Explicitly, HEK293 derived FlipIn-FM and FlipIn-RV dual reporter cells each include a single stable HIV-1 long terminal repeat (LTR)-driven luciferase reporter genes, with a second complimentary non-HIV (off-target) luciferase reporter. These cell lines model post integration latency and read-through transcription by expressing a low level of HIV-1 Tat protein via a native Internal Ribosome Entry Site (IRES) found within Tat, from a chimeric cellular-tat gene cassette. FlipIn-FM and FlipIn-RV clones were chosen for low basal levels of LTR activity and high responsiveness to Tat as well as many Latency Reversing Agents (LRAs).

The dual reporter cell lines contain three stably integrated constructs that together allow for detection of novel LRAs, capable of potent and specific HIV reactivation. The FlipIn.FM line contains a proviral LTR-driven nef/CBR reporter fusion gene, allowing for detection of viral gene expression. A second CMV-driven CBG luciferase reporter allows for detection of off target drug effects, including non-specific activation and drug toxicity. The third construct contains the first coding exon of HIV-1 tat, within human Growth Hormone (hGH) as a chimeric gene, expressing HIV-1 Tat protein from an IRES mechanism that underlies the tat exon. This construct models read-through transcription and the low level of Tat protein expression that occurs during post integration latency. The counter screening reverse cell line, FlipIn.RV, contains the same three components with the Click Beetle Luciferase genes in the opposite orientation (LTR-CBG and CMV-CBR) for counter screening.

Compounds were evaluated in an 11-point titration using normal CMV-CBG/LTR-CBR reporter cell line and an 11 point reverse CMV-CBR/LTR-CBG reporter cell line.

Compounds were also evaluated in a dose ranging titration with HIV latently infected cell lines (J.Lat 6.3 and 10.6). The specificity of the selected compounds in these cell line models, were measured by inserting a CMV-DS.Red reporter into these cells to co-ordinately measure HIV specific LTR-Green fluorescent protein and non-specific DS.Red expression during FACS analysis.

The FlipIn cell lines were therefore designed with a dual purpose, to detect novel compounds that reactivate HIV-1 and to also screen out compounds that behaved in a largely non-specific manner. To achieve the latter, the cell lines contain an “off target” reporter gene construct, driven by the unrelated CMV-IE promoter, that allows for the detection of drug mediated off target effects including global gene activation as well as possible toxicity.

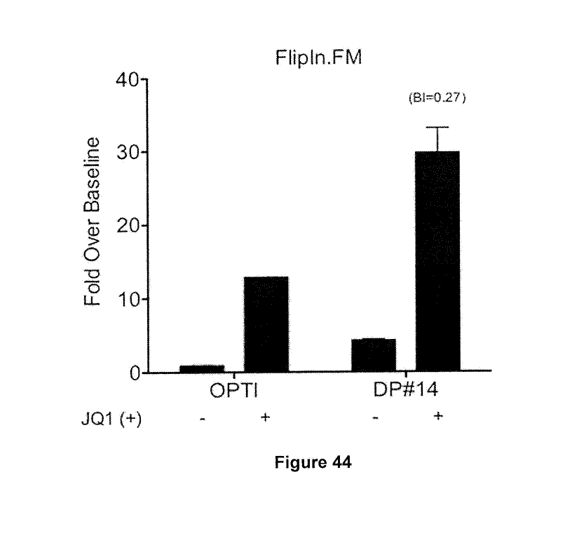

FIG. 44 shows the synergistic relationship between JQ1 (+) and DP#14 of Series E in the FlipIn.FM model of HIV-1 latency. At 10 μM, JQ1 (+) achieved a 12.8-fold change over the unstimulated baseline, and DP#14 achieved a 4.2-fold increase. In combination, the pair achieved a 29.7-fold increase over the baseline. Performing the Bliss Independence calculation of synergy gave a BI=0.27, demonstrating the pair to be synergistic.

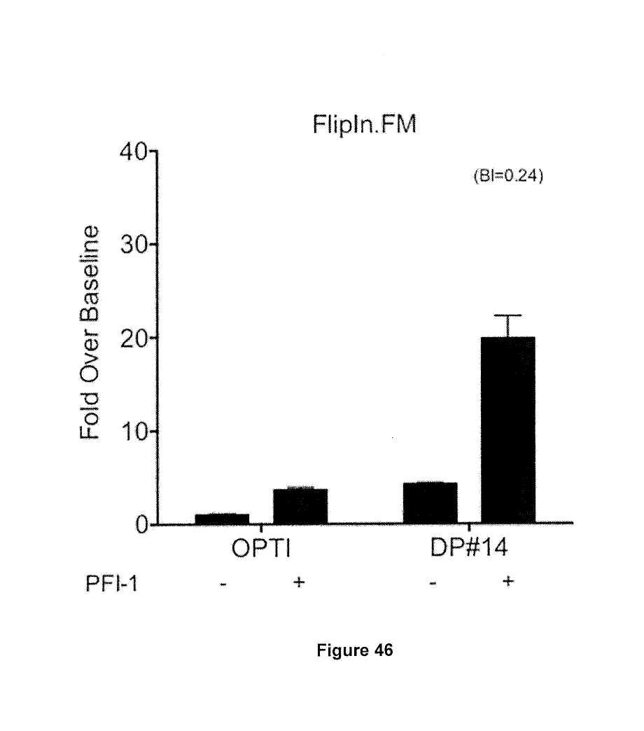

FIG. 46 shows the synergistic relationship between PFI-1 and DP#14 of Series E in the FlipIn.FM model of HIV-1 latency. At 10 μM, PFI-1 achieved a 3.6-fold change over the unstimulated baseline, and DP#14 achieved a 4.2-fold increase. In combination, the pair achieved a 19.8-fold increase over the baseline. Performing the Bliss Independence calculation of synergy gave a BI=0.24, demonstrating the pair to be synergistic.

Results of the assay for certain compounds of the present invention are shown in the following table:

| | Latent reporter | Global reporter vs latent reporter |
|---|---|---|
| Compound ID | Av EC50 (uM) | (equal to or >40 uM) |
| 40 | 6.86 | 40 |
| 41 | 14.0 | 40 |
| 1 | 4.88 | 40 |
| 4 | 9.26 | 40 |
| 5 | 3.48 | 40 |
| 6 | 1.09 | equal to |
| 7 | 1.64 | 40 |
| 8 | 1.99 | 40 |
| 12 | 3.22 | 40 |
| 14 | 3.09 | 40 |
| 15 | 2.56 | equal to |
| 16 | 1.23 | equal to |
| 17 | 2.83 | equal to |
| 18 | 2.83 | equal to |
| 19 | 1.48 | equal to |
| 20 | 1.85 | 40 |
| 21 | 2.12 | equal to |
| 22 | 2.01 | 40 |
| 29 | 7.6 | 40 |
| 30 | 9.74 | equal to |
| 36 | 4.20 | equal to |
| 38 | 5.07 | 40 |
| 58 | 2.0 | equal to |
| 57 | 1.7 | equal to |
| 82 | 2.0 | equal to |
| 52 | 3.80 | equal to |
| 61 | 4.29 | 40 |
| 54 | 19.4 | equal to |
| 59 | 1.11 | equal to |
| 51 | 2.37 | equal to |
| 62 | 4.74 | equal to |
| 60 | 3.56 | equal to |
| 55 | 17.9 | equal to |
| 44 | 5.3 | equal to |
| 47 | 6.55 | equal to |
| 46 | 5.23 | equal to |
| 42 | 1.42 | equal to |
| 56 | 14.8 | equal to |
| 48 | 4.13 | equal to |
| 49 | 2.64 | equal to |
| 45 | 5.29 | equal to |
| 43 | 12.5 | 40 |
| Piperazines | | |
| 63 | 0.45 | equal to |
| 64 | 0.22 | equal to |
| 65 | 0.33 | equal to |
| 66 | 0.58 | equal to |
| 67 | 0.27 | equal to |
| 68 | 0.52 | equal to |
| 69 | 0.34 | equal to |
| 70 | 0.21 | equal to |
| 71 | 0.88 | equal to |
| 72 | 0.25 | equal to |
| 73 | 0.28 | equal to |
| Homopiperazines | | |
| 74 | 5.2 | equal to |
| 75 | 5.46 | equal to |
| Amidothiazole | | |
| isosteres | | |
| 76 | 7.75 | equal to |
| 77 | 3.97 | equal to |
| Piperidines | | |
| 78 | 11.4 | equal to |
| 79 | 3.83 | equal to |
| Pyrrolidines | | |
| 80 | 10.1 | equal to |
| 81 | 1.5 | equal to |
| other | | |
| 83 | 2.2 | 40 |
| 82 | 5 | equal to |

The HIV-LTR driven latent reporter gene identifies the HIV-specific activation, and the term “global reporter” is interchangeable with “off target reporter” and refers to the unrelated CMV-IE promoter driven reporter gene, which is used as a surrogate for global gene activation. We performed 11-point, 2-fold dilution series experiments and the EC₅₀ values were derived and tabulated above. The highest concentration tested here was 40 μM. If a drug did not induce activation of the CMV “off target reporter” in these experiments even at the highest dose of 40 μM, and thereby did not display any notable off target effects. These compounds were specific for the HIV component and were assigned a >40 value in the table above. However, if a compound showed any measurable off target effects, they are described as “equal to”, indicating the off target promoter was induced in addition to that of the HIV component.

### Biological Assay—J.Lat Model

The J.Lat model of HIV-1 latency is a well-established model used widely and is described in detail in the following paper:

HIV reproducibly establishes a latent infection after acute infection of T cells in vitro, Eric Verdin et al, The EMBO Journal Vol. 22 No. 8 pp 1868-1877, 2003

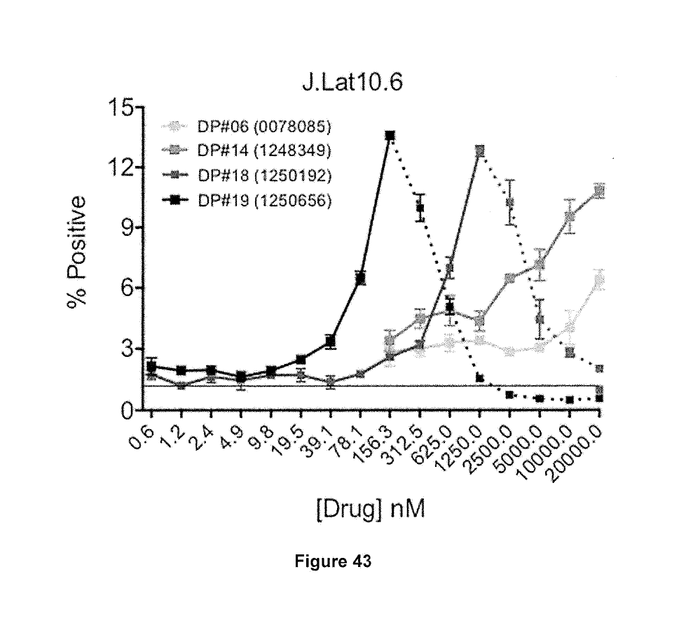

FIG. 43 shows the progression of compounds of the present invention, with a marked increase in their ability to reactivate HIV-1 gene expression within the J.Lat10.6 T-cell line. Original library hit DP#6 (WECC-0078085) showed an IC₅₀ value of approximately 16.5 μM. The first round of analogues yielded DP#14 (WEHI-1248349), which increased the potency to an IC₅₀ value of approximately 4.5 μM. Subsequent medicinal chemistry further increased the potency to IC₅₀=0.65 μM for DP#18 (WEHI-1250191), the third generation compound, and again to IC₅₀<0.11 μM for DP#19 (WEHI-1250656) in generation four. Overall, medicinal chemistry has seen close to a 2-log reduction in the IC₅₀ values within Series E.

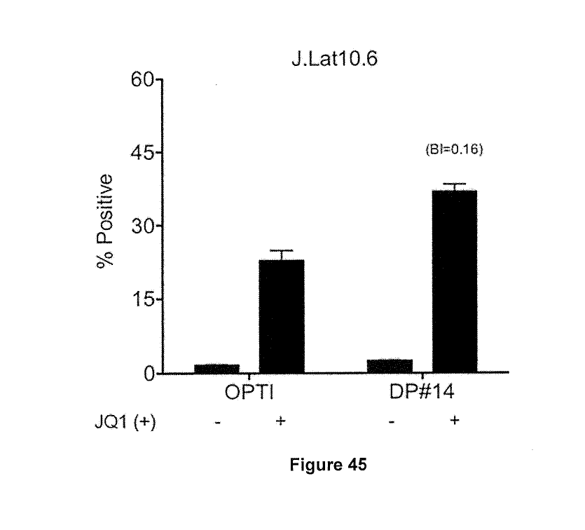

FIG. 45 shows the synergistic relationship between JQ1 (+) and DP#14 of the present invention in the J.Lat10.6 model of HIV-1 latency. At 10 μM, JQ1 (+) reactivated HIV-1 gene expression in 22.8 percent of the cells treated, and DP#14 reactivated 2.4 percent. In combination, the pair reactivated 36.8 percent of the cells treated. Performing the Bliss Independence calculation of synergy gave a BI=0.16, demonstrating the pair to be synergistic.

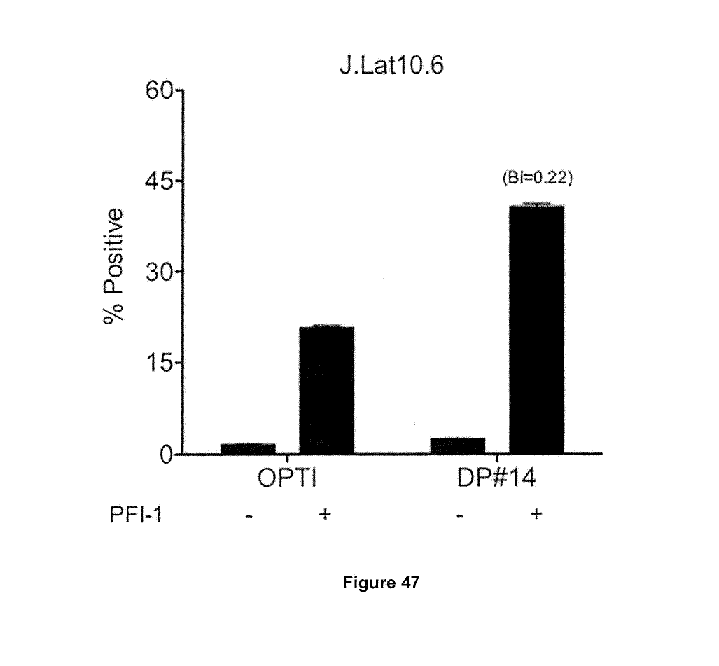

FIG. 47 shows the synergistic relationship between PFI-1 and DP#14 of the present invention in the J.Lat10.6 model of HIV-1 latency. At 10 μM, PFI-1 reactivated HIV-1 gene expression in 20.6 percent of the cells treated, and DP#14 reactivated 2.4 percent. In combination, the pair reactivated 40.6 percent of the cells treated. Performing the Bliss Independence calculation of synergy gave a BI=0.22, demonstrating the pair to be synergistic.

The inclusion of a piprazine motif within the structure of Series E in generations 3 and 4, while increasing the potency of the series substantially, also introduced notable toxicity at concentration above 1.25 μM (DP#18) and 156 nM (DP#19). This dose dependent toxic effect, however, was not seen in DP#6 and DP#14, which could be dosed as high as 40 μM and show no such toxicity.

The increased toxicity in DP#18 and DP#19 by no means indicates that these compounds are not useful in this and other applications. In certain circumstances, particular dosing regimes or coadministration of other drugs can mitigate this side effect.

Leukapharesis—Materials and Methods

A. Isolation of CD4+ T Cells Form Leukapheresis Samples

A leukapheresis apparatus was used to collect lymphocytes from individual HIV-infected volunteers on combination ART who each had fully suppressed viral loads that were below the limit of detection (50 vRNA copies per ml of blood). Total peripheral blood mononuclear cells (PBMC) were stored frozen in liquid nitrogen and prior to use, vials of frozen cells (0.5×10⁸ PBMCs/vial or 1×10⁸ PBMCs/vial) were quick thawed in a 42° C. water bath. Cells were promptly transferred to a 15 mL tube with 5 mL FBS dropwise then, then 6 mL of RF10 was added. Cells were pelleted at 300 g for 10 min at room temperature. Following aspiration, the cells were resuspended in RF10, pooled into a 50 mL tube which was topped up with RF10. PBMC were pelleted again at 300 g for 10 min at room temperature. Following aspiration, the cells were resuspended in PBS(−/−) and counted. CD4+ T cells were then isolated from 4×10⁸ PBMCs that were pelleted and resuspended in PBS(−/−) at 1×10⁷ cells/40 μL. 10 μL of CD4+ T cell Biotin-Antibody cocktail (Miltenyi Biotec) for every 1×10⁷ cells was added and the mix refrigerated for 5 min. 30 μL of PBS(−/−) for every 1×10⁷ cells was added, and 20 μL of the CD4+ T cell MicroBeads for every 1×10⁷ cells was added and the mix refrigerated for 10 min. Unlabelled CD4+ T cells were then isolated by negative selection using magnetic separation. CD4+ T cells were then counted before being diluted to 4×10⁶ cells/500 μL in RF10 for each condition and seeded into a 48 well plate.

B. Reactivation of Latent HIV from Leukapheresis Samples

Latent HIV was reactivated in the presence of the HIV integrase inhibitor Raltagravir (Ral) to prevent any further rounds of infection. Ral was made to [2 μM] in RF10+IL-2 (2 U/mL), and used to make 1 mL preparation of each drug up at [×2]. 500 μL of the [×2 Ral/IL-2/drug] was then added to the appropriate wells containing cells. Cells were transported to PC3 and incubated for 72 hrs.

C. Harvesting HIV Reactivated from Leukapheresis Cells

Following reactivation, 800 μL of cell supernatant was transferred to labeled 1.5 mL screwcap tubes, pelletted at 800 g for 10 min then transferred to a second set of tubes and frozen for possible use at a later date. 800 μL of PBS(−/−) was added to each well to mix the cells and 50 μL of cells transferred to another set of tubes for live/dead staining and flow cytometry. Cells were pelleted at 400 g for 10 min, the media aspirated and cells resuspended in 100 μL of a [×1] APC-cy7 live/dead stain (Life technologies). Cells were then incubated for 30 min protected from light. Following staining, cells were washed twice with PBS (−/−) and resuspended in 100 μL FACS FIX for flow cytometry analysis.

The remaining 950 μL cells were pelleted at 400 g for 10 min, the supernatant aspirated and the cells resuspended in 750 μL of TRIzol® for Phenol Chloroform RNA extraction and precipitation in 80% v/v ethanol. RNA pellets were resuspended in 40 μL RNase free water.

D. DNase Treatment of Whole RNA

4 μL of RQ1 DNase and 4 μL×10 buffer added to 40 μL RNA and incubated at 37° C. for 30 min. 2 μL of the DNase stop solution was added and incubated at 65° C. for 10 min.

E. cDNA Synthesis

Reverse Transcription PCR setup.
First strand cDNA synthesis

| dNTP [10 mM] | 2 μL |
|---|---|
| Random Hexamer [36 ng/μL] | 2 μL |
| Oligo(dT)₂₀ [0.5 μg/μL] | 1 μL |
| Whole cell RNA | 1000 ng (XμL) |
| DNAse free H₂O | To 26 μL |
| 65° C. 5 min then on ice 1 min | |
| First Strand Buffer ×5 | 8 μL |
| DTT [0.1 M] | 2 μL |
| RNase Inhibitor | 2 μL |
| Superscript III RT | 2 μL |

50° C. 60 min, 70° C. 15 min

F. First Round PCR for MS HIV DNA Amplification

Where needed, amplification of multiply spliced (MS) HIV DNA was promoted by a first round PCRs using the Amplitaq Gold® system (ThermoFisher) as follows: 95° C. for 10 min to allow DNA melting, then 15 cycles of 94° C. for 10 sec, 58° C. for 20 sec, 72° C. for 20 sec. Final elongation was allowed 5 min at 72° C. for completion.

First round PCR for MS template.
Multiply Spliced HIV DNA

| | |
|---|---|
| Buffer II | 10 μL |
| dNTP [10 mM] | 1 μL |
| MgCl₂ [50 mM] | 0.5 μL |
| Phusion | 0.5 μL |
| Polymerase | |
| SL28 [20 μM] | 0.5 μL |
| TM1 [20 μM] | 0.5 μL |
| DNAse free H₂O | 32 μL |
| cDNA | 5 μL |

G. qPCR of HIV DNA

Amplified first round HIV DNA, or cDNA for US HIV DNA was used as a template for qPCR using the Brilliant II SYBR® Green qPCR system as follows: 95° C. for 10 min to allow DNA melting, then 60 cycles of 94° C. for 20 sec, 58° C. for 20 sec, 72° C. for 20 sec. Dissociation curves were generated by increasing the temperature from 600° C. to 90° C. at a rate of 0.5° C./read

TABLE 3
qPCR for HIV DNA.

| Multiply Spliced HIV DNA | | Unspliced HIV DNA | |
|---|---|---|---|
| SYBR mix | 10 μL | SYBR mix | 10 μL |
| Odp3113 [20 μM] | 0.5 μL | Odp3063 [20 μM] | 0.5 μL |
| Odp3114 [20 μM] | 0.5 μL | Odp3064 [20 μM] | 0.5 μL |
| DNAse free H₂O | 7 μL | DNAse free H₂O | 7 μL |
| First round DNA | 2 μL | First round DNA | 2 μL |

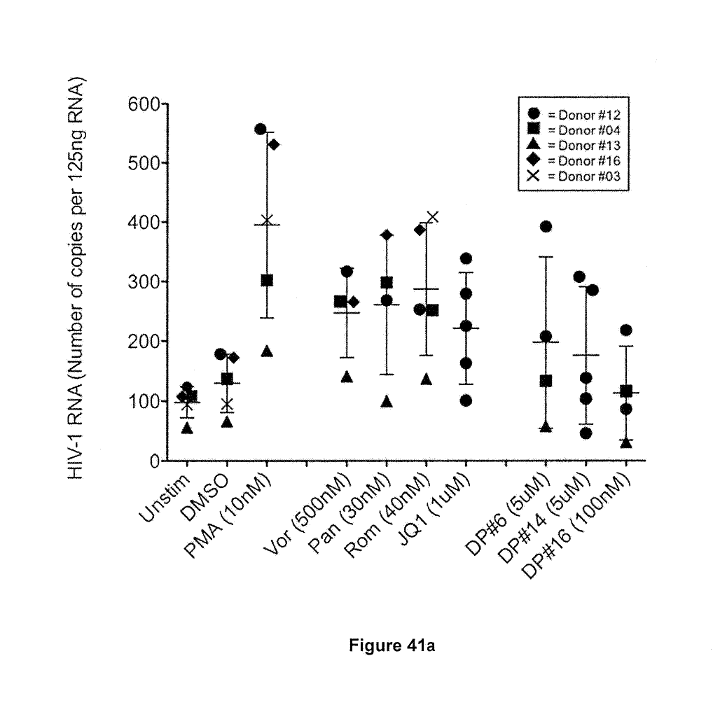

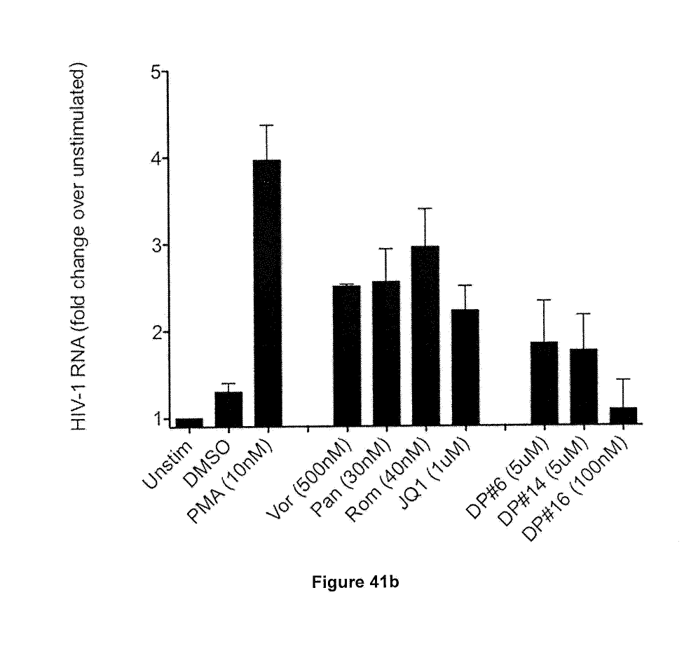

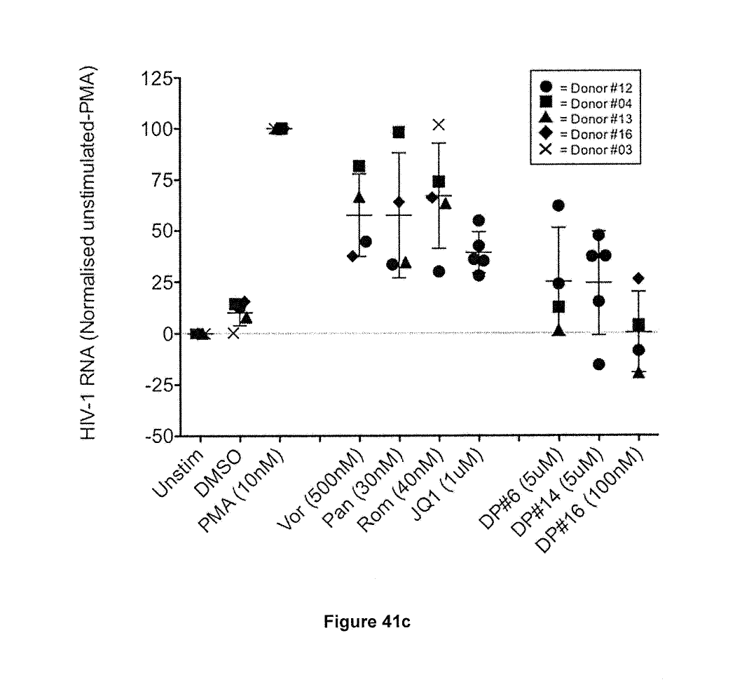

The FIG. 41 set shows the same data set represented as a) absolute number of HIV-1 RNA molecules per 125 ng of whole RNA, b) Fold change in induction over the unstimulated baseline and c) values normalized between the unstimulated negative and PMA stimulated positive controls. The HDACi compounds were included as an additional set of controls as their behavior in similar experiments has been reported previously. Three compounds of the present invention were chosen, spanning the first three generations DP#6 (WECC0078085), DP#14 (WEHI-1248349) and DP#16 (WEHI-1250191). We see a modest induction of HIV-1 gene expression form leukapheresis samples with the HDACi compounds Vorinostat, Panabinostat and Romidepsin, achieving 58%, 57% and 67% of the normalized values respectively. Bromodomain inhibitor JQ1 (+) achieved a 39% induction. For the compounds of the present invention, DP#6 and DP#14 achieved a 25% and 24% induction respectively at 5 μM, where DP#16, at a lower concentration of 100 nM achieved activation in only one patient.

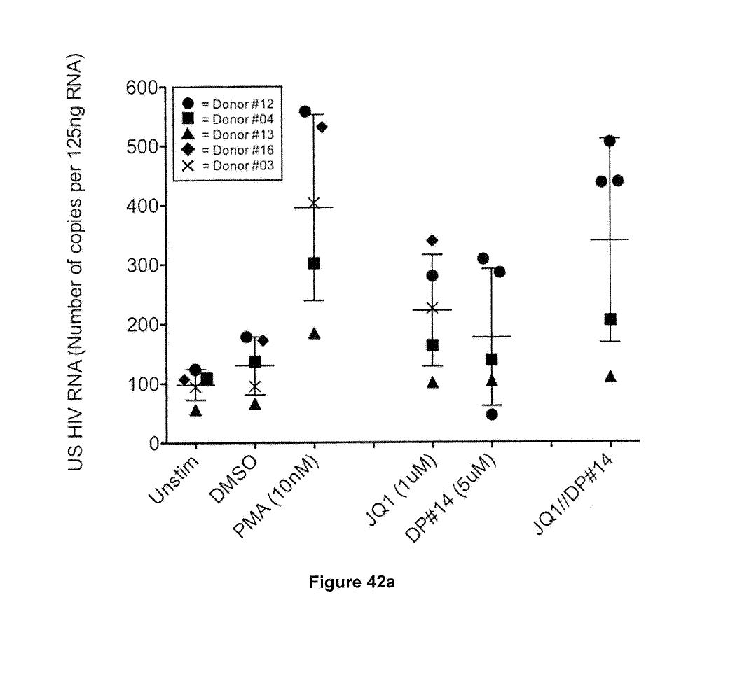

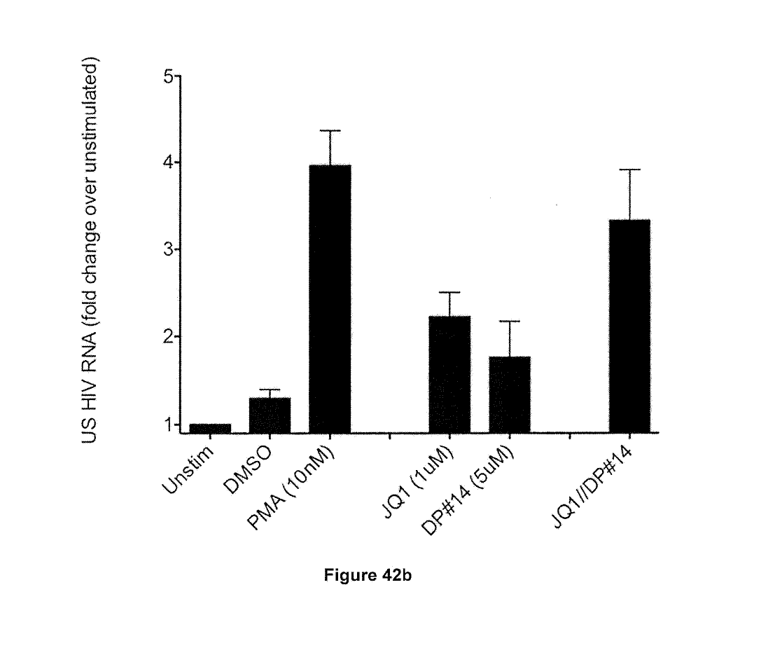

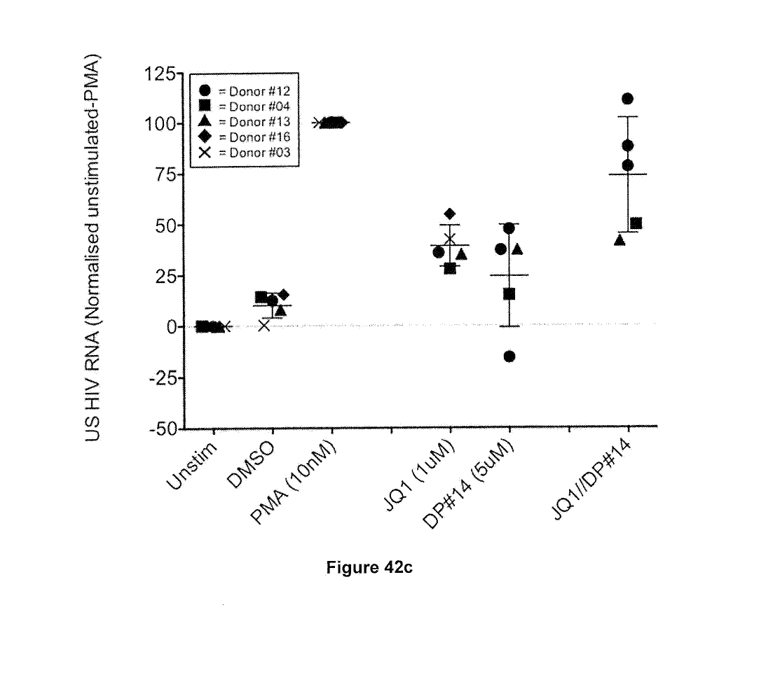

The FIG. 42 set shows the same data set represented again as a) absolute number of HIV-1 RNA molecules per 125 ng of whole RNA, b) Fold change in induction over the unstimulated baseline and c) values normalized between the unstimulated negative and PMA stimulated positive controls. For this study DP#14 (WEHI-1248349) was used in combination with the Bromodomain inhibitor JQ1 (+). Alone JQ1 (+) was able to achieve an induction of 39% of the normalized value, and DP#14 alone achieved 24%. In combination together however, the JQ1 (+)/DP#14 pair achieved an induction of 74% of the normalize value.

Calculating the synergy between JQ1 (+) and DP#14 using the Bliss Independence method follows:

F_{JQ1(+)}=0.39

F_{DP#}14=0.24

F_{Observed}=0.74

F_{Predicted}=F_{JQ1 (+)}+F_{DP#}14−(F_{JQ1 (+)}×F_{DP#}14)

F_{Predicted}=0.39+0.24−(0.39×0.24)

F_{Predicted}=0.54

BI=F_{Observed}−F_{Predicted}

BI=0.74−0.54

BI=0.2

A BI value greater than 0 indicates a synergistic relationship between JQ1 (+) and DP#14 in these leukapheresis experiments.

Biological Activity

The data show that compounds of the present invention are selective for HIV and reactivate HIV latency. The compounds exhibit low levels of global gene-activation and cellular toxicity. These compounds may be used to eliminate long lived forms of virus that persist in HIV-infected patients on antiretro viral therapy (ART). Specifically compounds of the invention may be used to make HIV visible allowing for virus induced cytolysis, or immune mediated clearance, and/or lockdown or to permanently suppress latent HIV.

It will be understood that the invention disclosed and defined in this specification extends to all alternative combinations of two or more of the individual features mentioned or evident from the text or drawings. All of these different combinations constitute various alternative aspects of the invention.

### REFERENCES

P. H. Stahl, C. G. Wermuth, “Handbook of Pharmaceutical salts” 1st edition, 2002,
Wiley-VCH.
Martindale—The Extra Pharmacopoeia (Pharmaceutical Press, London 1993) and Martin (ed.), Remington's Pharmaceutical Sciences.
Deeks, S. G., Lewin, S. R. & Havlir, D. V. The end of AIDS: HIV infection as a chronic disease. Lancet 382, 1525-33 (2013).
Lewin, S. R., Deeks, S. G. & Barre-Sinoussi, F. Towards a cure for HIV—are we making progress? Lancet 384, 209-11 (2014).
Archin, N. M. et al. Administration of vorinostat disrupts HIV-1 latency in patients on antiretroviral therapy. Nature 487, 482-5 (2012).
Elliott, J. H. et al. Activation of HIV Transcription with Short-Course Vorinostat in HIV-Infected Patients on Suppressive Antiretroviral Therapy. PLoS Pathog 10, e1004473 (2014).

1. A compound of Formula (I): 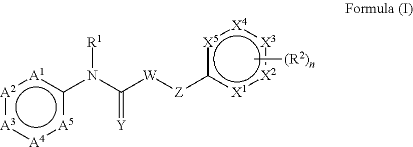
or a salt, solvate, or prodrug thereof
wherein
A¹, A², A³, A⁴ and A⁵ are independently selected from the group consisting of CR′, NR″, O and S, wherein A⁵ may or may not be present;
R′ is selected from the group consisting of H, C₁-C₄alkyl, O(C₁-C₄alkyl), CONR⁵R⁶, halo, CF₃, CF₂H and CN;
R″ is selected from H and C₁-C₄alkyl, wherein R″ may or may not be present;
R¹ is selected from H and C₁-C₄alkyl;
Y is selected from O and NH;
wherein when Y is NH and A⁵ is CH, optionally Y and A⁵ together form an imidazole ring so that the compound has the structure: 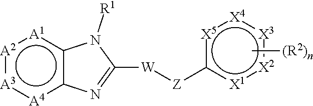
W is selected from the group consisting of C₁-C₄alkyl, NH, N(C₁-C₄alkyl) and 0;
Z is selected from the group consisting of C₁-C₄alkyl, (CH₂)ₘO, (CH₂)ₘNH(CH₂)ₘN(CH₃), and m is 0 or 1, wherein when W is O, m is 1;
alternatively W and Z together form an optionally substituted piperazine or piperidine ring so that the compound has the structure: 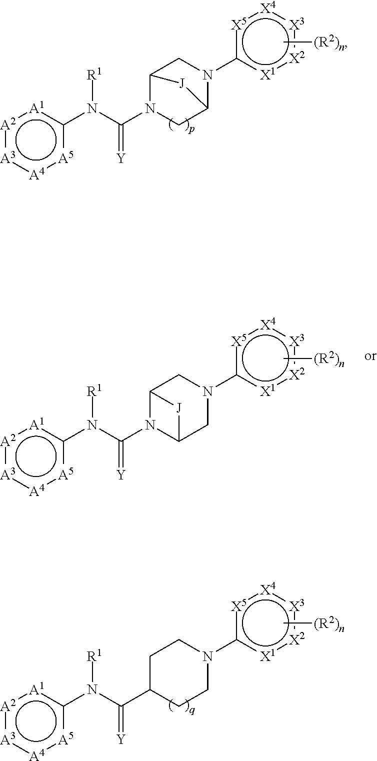
J is selected from CH₂ and (CH₂)₂, wherein J may or may not be present, p is 1 or 2, and q is 0 or 1;
X¹, X², X³, X⁴ and X⁵ are independently selected from the group consisting of CH, N, NH, O and S, wherein X⁵ may or may not be present;
each R² is independently selected from the group consisting of C₁-C₄alkyl, CN, CF₃, F, Cl, Br, hydroxyl, nitro, OR⁶, COR⁶, CO₂R⁶, CONR⁵R⁶, CONHSO₂R⁵, SO₂NHCOR⁵, CONR⁵OR⁶, C₁-C₄alkylNR⁵R⁶, C₁-C₄alkylOR⁶, NR⁵R⁶, NR⁵COR⁶, NR⁷CONR⁵R⁶ and NR⁵CO₂R⁶;
n is 0-3;
R⁵ and R⁶ are independently selected from the group consisting of H, C₁-C₄alkyl, C₃-C₁₀cycloalkyl, C₃-C₁₀heterocyclyl, C₆-C₁₀aryl, C₅-C₁₀heteroaryl, (C₁-C₄alkyl)C₆-C₁₀aryl and (C₁-C₄alkyl)C₅-C₁₀heteroaryl;
alternatively when R⁵ and R⁶ are bound to the same atom they form an optionally substituted C₃-C₁₀cycloalkyl or C₃-C₁₀heterocyclyl;
R⁷ is selected from H and CH₃.

2. The compound of claim 1, wherein A⁵ is not present so that the compound has the structure:

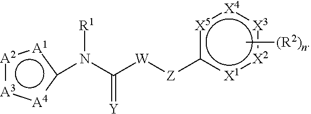

3. The compound of claim 1 or 2, wherein A¹ is selected from CH and N.

4. The compound of claim 3, wherein A¹ is N.

5. The compound of any one of the preceding claims, wherein A² is selected from CH, N, N(CH₃), and 0.

6. The compound of claim 5, wherein A² is CH.

7. The compound of any one of the preceding claims, wherein A³ is selected from CH, C(CH₃), C(CH₂CH₃), C(Br), C(Cl), C(CN), C(CF₃), and N(CH₃).

8. The compound of claim 7, wherein A³ is selected from C(CH₃), C(Br), C(Cl) and C(CN).

9. The compound of claim 8, wherein A³ is C(CH₃).

10. The compound of any one of the preceding claims, wherein A⁴ is selected from S, O, CH, and NH.

11. The compound of claim 10, wherein A⁴ is S.

12. The compound of any one of claims 1 or 3-11, wherein A⁵ is CH.

13. The compound of any one of the preceding claims, wherein A′, A², A³, A⁴ and A⁵ form a ring which does not include 2 heteroatoms adjacent to one another.

14. The compound of claim 13, wherein the ring does not include 2 nitrogen heteroatoms adjacent to one another.

15. The compound of claim 13, wherein the ring does not include a nitrogen heteroatom and an oxygen heteroatom adjacent to one another.

16. The compound of any one of the preceding claims, wherein R¹ is H.

17. The compound of any one of the preceding claims, wherein Y is O.

18. The compound of any one of the preceding claims, wherein W is C₁-C₄alkyl.

19. The compound of claim 18, wherein W is (CH₂)₂.

20. The compound of any one of the preceding claims, wherein Z is selected from C₁-C₄alkyl and (CH₂)ₘO.

21. The compound of claim 20, wherein Z is selected from CH₂, (CH₂)₂ and (CH₂)O.

22. The compound of claim 21, wherein Z is (CH₂)O.

23. The compound of any one of the preceding claims, wherein X¹, X², X³, and X⁴ are each CH.

24. The compound of any one of the preceding claims, wherein X⁵ is CH.

25. The compound of any one of the preceding claims, wherein each R² is independently selected from the group consisting of Br, Cl, CH₃, CF₃, and CN.

26. The compound of claim 25, wherein each R² is independently selected from Br and Cl.

27. The compound of any one of the preceding claims, wherein n is 2.

28. The compound of claim 27, wherein R² is located at positions 3 and 4, so that the compound is of the form

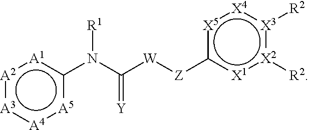

29. The compound of claim 1, selected from the group consisting of:

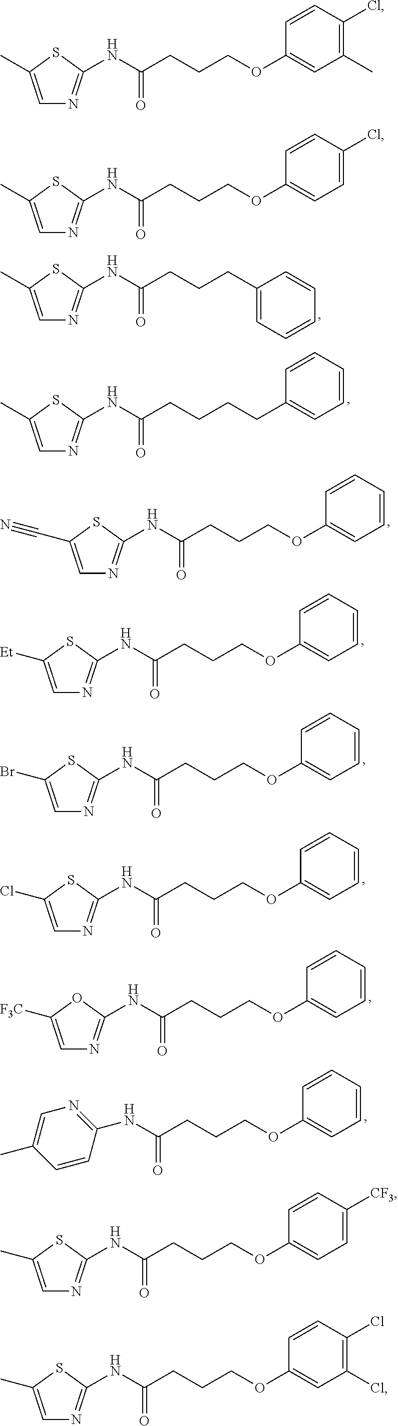

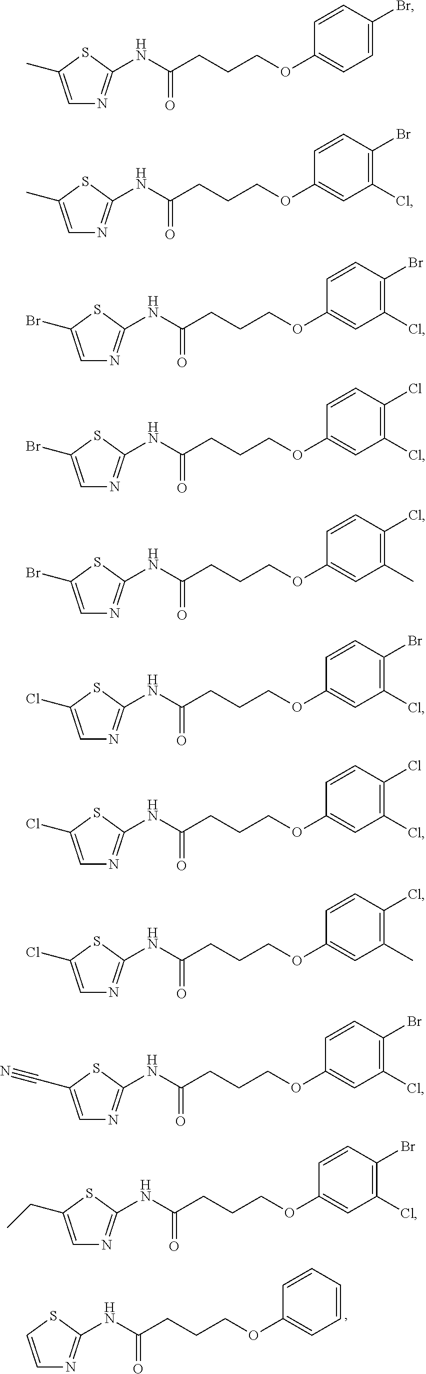

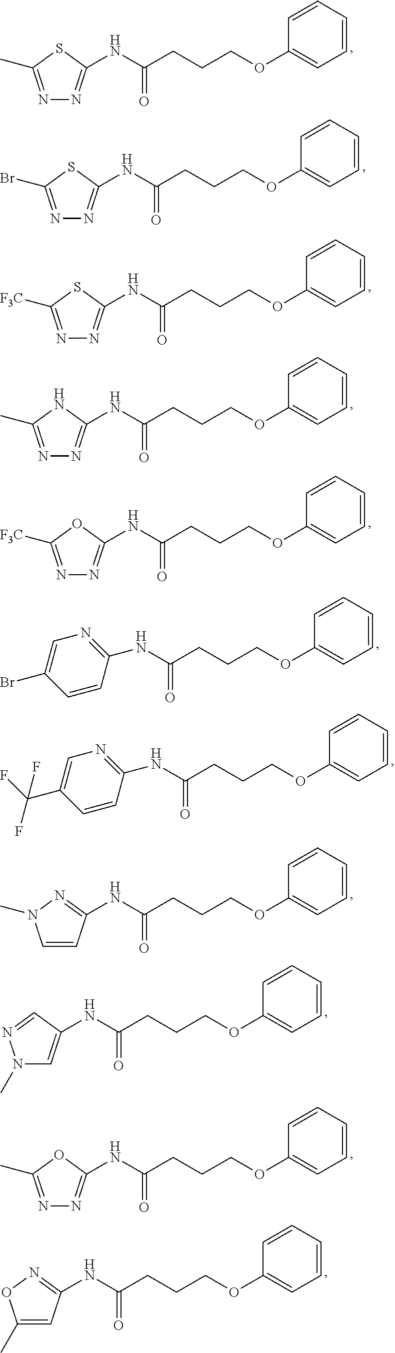

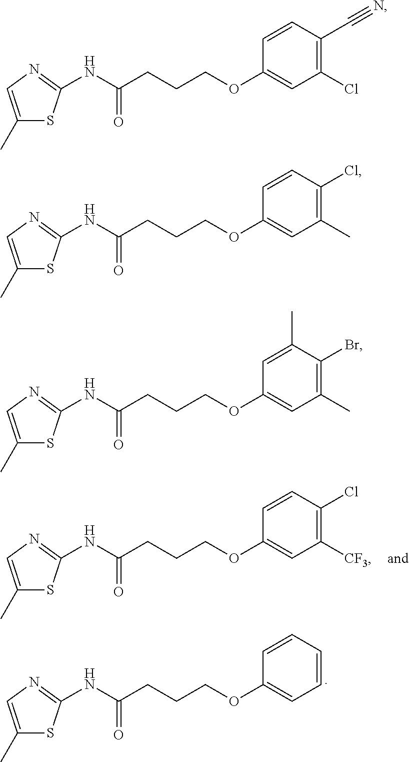 [SMILES: Cc1cnc(NC(=O)CCCOc2ccc(C#N)c(Cl)c2)s1 ; Cc1cnc(NC(=O)CCCOc2ccc(Cl)c(C)c2)s1 ; Cc1cnc(NC(=O)CCCOc2cc(C)c(Br)c(C)c2)s1 ; Cc1cnc(NC(=O)CCCOc2ccc(Cl)c(C(F)(F)F)c2)s1 ; Cc1cnc(NC(=O)CCCOc2ccccc2)s1]

30. The compound of claim 29, selected from the group consisting of:

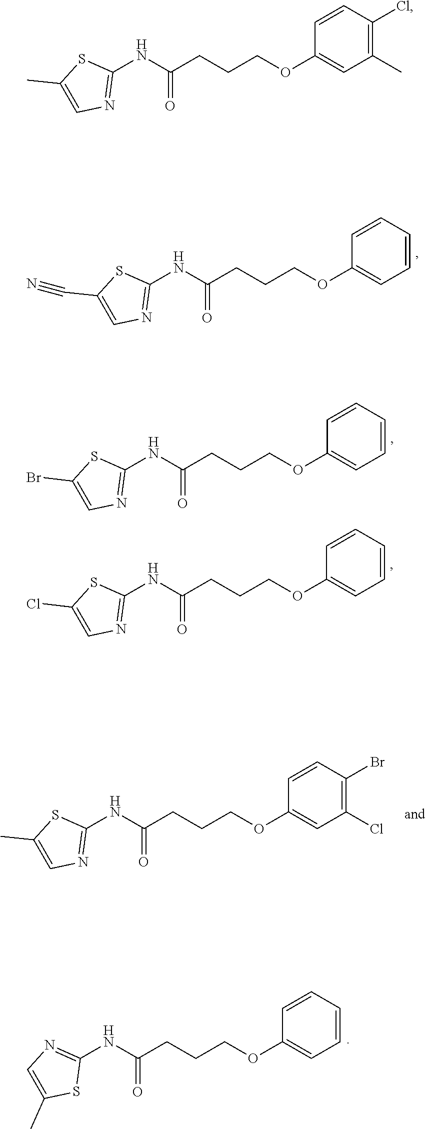

31. The compound of claim 1, provided the compound is not selected from the group consisting of:

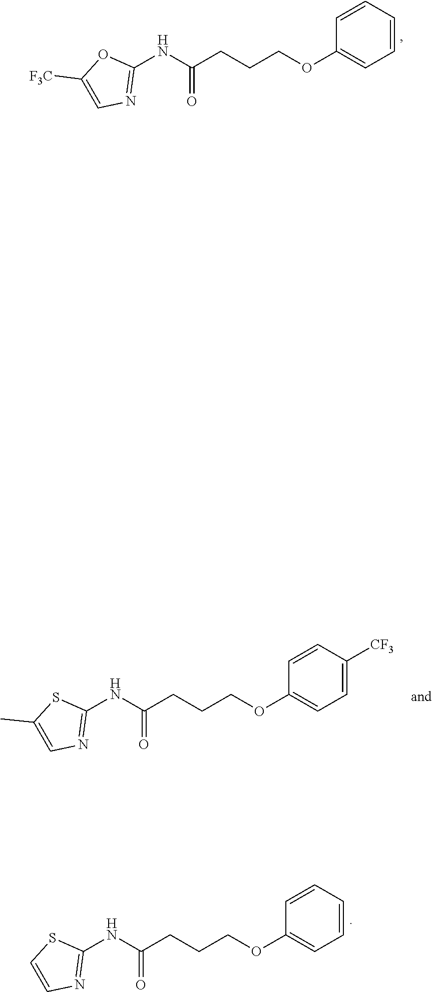 [SMILES: O=C(CCCOc1ccccc1)Nc1ncc(C(F)(F)F)o1 ; Cc1cnc(NC(=O)CCCOc2ccc(C(F)(F)F)cc2)s1 ; O=C(CCCOc1ccccc1)Nc1nccs1]

32. A composition comprising the compound of any one of the preceding claims or a salt, solvate or prodrug thereof, and a pharmaceutically acceptable excipient.

33. A method for activating HIV expression in latently infected cells in a subject in need thereof, the method comprising administering an effective amount of a compound of any one of claims 1-31 or a salt, solvate, or prodrug thereof; or a composition according to claim 32 to the subject.

34. A method for treating HIV infection in a subject in need thereof, the method comprising administering an effective amount of a compound of any one of claims 1-31 or a salt, solvate, or prodrug thereof; or a composition according to claim 32, in combination with a therapeutically effective amount of one or more anti-HIV viral therapy compounds to the subject.

35. A method according to claim 33 or 34 wherein the compound or composition is administered in combination with a bromodomain inhibitor.

36. A method according to claim 35 wherein the bromodomian inhibitor is JQ1.

37. Use of a compound of any one of claims 1-31 or a salt, solvate, or prodrug thereof; or a composition according to claim 32 for activating HIV expression in latently infected cells in a subject in need thereof.

38. Use of a compound of any one of claims 1-31 or a salt, solvate, or prodrug thereof; or a composition according to claim 32, in combination with one or more anti-HIV viral therapy compounds for treating HIV infection in a subject in need thereof.

39. A use according to claim 37 or 38 wherein the compound or composition is administered in combination with a bromodomain inhibitor.

40. A use according to claim 39 wherein the bromodomian inhibitor is JQ1.41.A compound according to any one of claims 1-31 or a salt, solvate, or prodrug thereof; or a composition according to claim 32 for use in activating HIV expression in latently infected cells in a subject in need thereof.

42. A compound according to any one of claims 1-31 or a salt, solvate, or prodrug thereof; or a composition according to claim 32, in combination with one or more anti-HIV viral therapy compounds for use in treating HIV infection in a subject in need thereof.

43. A compound or composition according to claim 41 or 42 wherein the compound or composition is administered in combination with a bromodomain inhibitor.

44. A compound or composition according to claim 43 wherein the bromodomian inhibitor is JQ1.

45. A compound according to any one of claims 1-31 or a salt, solvate, or prodrug thereof; or a composition according to claim 32, when used for activating HIV expression in latently infected cells in a subject in need thereof.

46. A compound according to any one of claims 1-31 or a salt, solvate, or prodrug thereof; or a composition according to claim 32, in combination with one or more anti-HIV viral therapy compounds when used for treating HIV infection in a subject in need thereof.

47. A compound or composition according to claim 45 or 46 wherein the compound or composition is administered in combination with a bromodomain inhibitor.

48. A compound or composition according to claim 35 wherein the bromodomian inhibitor is JQ1.
